(19) United States
(12) Patent Application Publication
(10) Pub. No.: US 2024/0225681 A1
(43) Pub. Date: Jul. 11, 2024

(54) SOFT TISSUE CUTTING DEVICES, SYSTEMS, AND METHODS

(71) Applicant: Sonex Health, Inc., EAGAN, MN (US)

(72) Inventors: DARRYL E. BARNES, EAGAN, MN (US); JAY SMITH, INVER GROVE HEIGHTS, MN (US); DAVID HERRIDGE, MENDOTA HEIGHTS, MN (US); BRIAN MULLINS, EAGAN, MN (US); RICK KOGLER, EAGAN, MN (US); GRACE RIERMANN, EAGAN, MN (US)

(73) Assignee: Sonex Health, Inc., EAGAN, MN (US)

(21) Appl. No.: 18/409,252

(22) Filed: Jan. 10, 2024

Related U.S. Application Data
(60) Provisional application No. 63/438,173, filed on Jan. 10, 2023.

(51) Int. Cl. A61B 17/32; A61B 17/00; A61B 17/02

(52) U.S. Cl. CPC A61B 17/320036; A61B 2017/0042; A61B 2017/00557; A61B 2017/0225

(57) ABSTRACT

Device, systems, and method for cutting body tissue may include a handle, a blade, a shaft, and an expander. The handle may include a slider and a lever in communication with the slider. The blade may be coupled with the slider. The shaft may extend distally from the handle and the shaft may define a guideway along which the blade is configured to travel. The expander may be in communication with the lever and extend along the shaft. Actuation of the lever may adjust the expander to an expanded position and unlock the slider to allow actuation of the slider and longitudinal movement of the blade along the guideway. The expander may be or may include one or more balloons.

### CROSS REFERENCE TO RELATED APPLICATIONS

This application claims the benefit of U.S. Provisional Patent Application No. 63/438,173, filed Jan. 10, 2023, the disclosure of which is incorporated herein by reference in its entirety for any and all purposes.

### TECHNICAL FIELD

The present disclosure relates to medical devices and methods. More specifically, the present disclosure relates to devices and methods for performing soft tissue cutting procedures.

### BACKGROUND

Capral tunnel syndrome is a condition that causes numbness, tingling, and/or weakness in the hand. The condition occurs when pressure is placed on the median nerve over long periods of time, typically by the transverse carpal ligament. The median nerve runs the length of the arm, passes through a passage in the wrist called the carpal tunnel, and ends in the palm of the hand.

Carpal tunnel syndrome affects approximately 3.7% of the general population and up to 7% of manual labor workers. Some cases of carpal tunnel syndrome are treated with a surgical carpal tunnel release procedure (“CTR procedure”). The standard CTR procedure involves forming an incision on the palm side of the hand, near the wrist, then finding and cutting the transverse carpal ligament to reduce median nerve compression and carpal tunnel pressures in the carpal tunnel region. Although the standard CTR procedure is highly effective, it can result in relatively long recovery times, due to the difficulty of fully healing the incision on a portion of the hand that moves frequently (i.e., the palm). Thus, a number of less invasive CTR devices and methods have been developed.

Examples of less invasive devices used for CTR surgery are described in U.S. Pat. Nos. 10,357,272, 11,259,829, and 10,864,055, the full disclosures of which are hereby incorporated by reference. In some configurations, the devices described in the above-referenced patents are used with external ultrasound visualization of the carpal tunnel region and the hand, to find and cut the transverse carpal ligament while avoiding nearby nerves and blood vessels.

### BRIEF SUMMARY

This disclosure provides design, material, manufacturing method, and use alternatives for performing soft tissue cutting procedures. Although it is noted that devices, systems, and methods for performing soft tissue cutting procedures are known, there exists a need for improvement on those approaches and systems.

In one example, a device for cutting body tissue may include a handle including a first actuator and a second actuator in communication with the first actuator, a blade coupled with the second actuator, a shaft extending distally from the handle, the shaft defining a guideway along which the blade is configured to travel, and an expander in communication with the first actuator and extending along the shaft, wherein actuation of the first actuator may adjust the expander to an expanded position and unlocks the second actuator to allow actuation of the second actuator and longitudinal movement of the blade along the guideway.

Alternatively or additionally to any of the embodiments in this section, in response to forces applied to the first actuator, the first actuator may adjust between an unactuated position in which the expander is in a relaxed position and a fully actuated position in which the expander is in the expanded position.

Alternatively or additionally to any of the embodiments in this section, the first actuator may be biased toward the unactuated position and the first actuator is configured to remain in the fully actuated position after the first actuator is adjusted to the fully actuated position and the forces applied to the first actuator are removed.

Alternatively or additionally to any of the embodiments in this section, the device may further include an expansion system at least partially located in the handle and in communication with the first actuator and the expander, wherein the expansion system is configured to cause the expander to expand in response to actuation of the first actuator.

Alternatively or additionally to any of the embodiments in this section, the expansion system may comprise a expansion device, a manifold in communication with the expansion device, and tubing in communication with the manifold and the expander, the expansion device being configured to cause fluid to flow through the manifold and the tubing to the expander in response to actuation of the first actuator.

Alternatively or additionally to any of the embodiments in this section, the handle may comprise a priming port in fluid communication with the expansion system and configured to engage a fluid source, the priming port including a one-way valve configured to open in response to the priming port engaging with the fluid source.

Alternatively or additionally to any of the embodiments in this section, the device may further include a linkage coupled with the first actuator and second actuator.

Alternatively or additionally to any of the embodiments in this section, the linkage may be coupled to the first actuator with an over-center mechanism configured to secure the first actuator in a fully actuated position until a force is applied to the first actuator to overcome the over-center mechanism.

Alternatively or additionally to any of the embodiments in this section, the device may further include a linkage extending between the first actuator and the first actuator, and the linkage may disengage from the second actuator when the first actuator is in a fully actuated position to unlock the second actuator and allow actuation of the second actuator to longitudinally move the blade along the guideway.

Alternatively or additionally to any of the embodiments in this section, the guideway may comprise a first portion with an incline, a second portion extending proximally from the first portion, and a third portion extending proximally from the second portion and having a decline.

Alternatively or additionally to any of the embodiments in this section, the shaft may have an elongate polymer portion defining the guideway and an elongate metal portion defining the guideway.

Alternatively or additionally to any of the embodiments in this section, the second actuator may be a second actuator and has a first extension and a second extension spaced proximally from the first extension.

In another example, a method of operating a device for cutting body tissue may include actuating a first actuator to radially enlarge an expander extending along a guideway for a blade of the device for cutting body tissue and unlock a second actuator coupled with the blade and adjusting the second actuator to move the blade from a protected first position to a cutting second position.

Alternatively or additionally to any of the embodiments in this section, the expander may be a balloon, and the method further include priming an expansion system with fluid, and wherein actuating the first actuator may actuate an expansion device of the expansion system causing the fluid to travel to the balloon and radially enlarge the balloon.

Alternatively or additionally to any of the embodiments in this section, priming the expansion system with fluid may include engaging a fluid source with a priming port of the device for cutting body tissue, creating a vacuum pressure in the expansion system and the fluid source by withdrawing a plunger of the fluid source; and releasing the plunger.

Alternatively or additionally to any of the embodiments in this section, the method may further include adjusting the second actuator to move the blade from the cutting second position to a protected third position.

In a further example, a handle assembly of a device for cutting body tissue may include a lever, an expansion system in communication with the lever, a slider configured to couple with a blade and longitudinally adjust the blade as the slider is longitudinally adjusted, and a linkage coupled with the lever and configured to limit movement of the slider, wherein when the lever is adjusted to a fully actuated position, the linkage may unlock the slider and allow the slider to adjust longitudinally.

Alternatively or additionally to any of the embodiments in this section, the assembly may further include a priming port in fluid communication with the expansion system.

Alternatively or additionally to any of the embodiments in this section, the expansion system may include a manifold in communication with the priming port;
tubing in fluid communication with the manifold and an expansion device configured to move fluid through the manifold and the tubing in response to adjustments of the lever.

Alternatively or additionally to any of the embodiments in this section, the manifold may include a pressure relief valve and a pressure relief chamber.

The above summary of some embodiments is not intended to describe each disclosed embodiment or every implementation of the present disclosure. The Figures, and Detailed Description, which follow, more particularly exemplify these embodiments.

### BRIEF DESCRIPTION OF DRAWINGS

The disclosure may be more completely understood in consideration of the following detailed description in connection with the accompanying drawings, in which:

FIG. 1 is a schematic perspective view of an illustrative configuration of a device for cutting soft tissue;

FIG. 2 is a schematic side view of the device for cutting soft tissue depicted in FIG. 1;

FIG. 3 is the schematic side view of the device for cutting soft tissue depicted in FIG. 2, with a portion of a housing removed;

FIG. 4 is a schematic perspective view of the device for cutting soft tissue depicted in FIG. 3;

FIG. 5 is a schematic side view of the device for cutting soft tissue depicted in FIG. 1, wherein the depicted side view is of a side of the device opposite the side depicted in FIG. 2 and has a portion of the housing removed;

FIG. 6 is a schematic cross-section view of an illustrative configuration of a handle portion of the device for cutting soft tissue depicted in FIG. 1;

FIGS. 7A and 7B are schematic views of an adjustment of an illustrative configuration of an expansion device of a device for cutting soft tissue;

FIG. 8 is a schematic side view of an illustrative configuration of a device for cutting soft tissue with a portion of a housing removed;

FIG. 9 is a schematic view of a portion of an illustrative configuration of an expansion system of a device for cutting soft tissue;

FIG. 10 is a schematic top view of a distal portion of the device for cutting soft tissue depicted in FIG. 1;

FIG. 11 is schematic side view of an illustrative configuration of a blade of a device for cutting soft tissue;

FIG. 12 is a schematic cross-section view of a distal portion of an illustrative configuration of a device for cutting soft tissue;

FIG. 13 is a schematic side view of a distal portion of the device for cutting soft tissue depicted in FIG. 1, with a portion of a shaft removed;

FIGS. 14A-14C are schematic views of an adjustment of a blade of a device for cutting soft tissue in response to movement of a slider of the device;

FIGS. 15A-15D are schematic perspective views of an illustrative technique for priming a device for cutting soft tissue;

FIGS. 16A-16E are schematic perspective view of an illustrative technique for adjusting a blade of a device for cutting soft tissue;

FIG. 17 is a schematic perspective view an illustrative device for cutting soft tissue;

FIG. 18 is a schematic perspective view an illustrative device for cutting soft tissue;

FIG. 19 is a schematic perspective view an illustrative device for cutting soft tissue;

FIG. 20 is a schematic perspective view an illustrative device for cutting soft tissue;

FIG. 21 is a schematic perspective view an illustrative device for cutting soft tissue;

FIG. 22 is a schematic perspective view an illustrative device for cutting soft tissue;

FIG. 23 is a schematic perspective view an illustrative device for cutting soft tissue;

FIG. 24 is a schematic perspective view an illustrative device for cutting soft tissue;

FIG. 25 is a schematic perspective view an illustrative device for cutting soft tissue;

FIGS. 26A-26C are schematic side views of an illustrative device for cutting soft tissue, with components of the device removed;

FIGS. 27A is a schematic perspective view of an illustrative device for cutting soft tissue, with components of the device removed;

FIG. 27B is a schematic magnified perspective view of the illustrative device for cutting soft tissue depicted in FIG. 27A, taken from circle 27B;

FIGS. 28A and 28B are a schematic perspective view of an illustrative device for cutting soft tissue, showing actuation of a first actuator;

FIGS. 29A and 29B are schematic side views of the devices depicted in FIGS. 28A and 28B, with a portion of the device removed.

FIG. 30 is a schematic perspective view of an illustrative device for cutting soft tissue;

FIG. 31 is a schematic perspective view of the device depicted in FIG. 30, with a portion of the device removed;

FIG. 32 is a schematic perspective view of an illustrative device for cutting soft tissue;

FIG. 33 is a schematic perspective view of the device depicted in FIG. 32, with a portion of the device removed;

FIGS. 34A and 34B are schematic perspective views of an illustrative handle of a device for cutting soft tissue, showing actuation of a first actuator.

FIGS. 35A-35D are schematic side, side, top, and magnified top views, respectively, of a portion of an illustrative device for cutting soft tissue;

FIG. 36 is a schematic perspective view of a portion of a distal end of an illustrative device for cutting soft tissue;

FIG. 37 is a schematic perspective view of a portion of a distal end of an illustrative device for cutting soft tissue;

FIG. 38 is a schematic cross-section view of a distal end of an illustrative device for cutting soft tissue, taken along line 38-38 of FIG. 36;

FIGS. 39A and 39B are schematic views of an illustrative configuration of a tube for use in a device for cutting soft tissue;

FIG. 40 is a schematic view of illustrative configurations of fluid openings in a tube for use in a device for cutting soft tissue;

FIG. 41 is a perspective view of a portion of an illustrative configuration of a shaft of a device for cutting soft tissue;

FIG. 42 schematically depicts three sets of balloons with micro-textured surfaces applied thereto;

FIGS. 43A-43C schematically depict views of different portions of material with different micro-textures;

FIG. 44 schematically depicts a material with a micro-texture;

FIG. 45 schematically depicts a side view of an illustrative configuration of a device for cutting soft tissue;

FIG. 46 schematically depicts a perspective view of an illustrative configuration of a blade shaft coupled with a blade;

FIG. 47 schematically depicts a perspective view of an illustrative configuration of a shaft of the device for cutting soft tissue with the blade shaft and blade depicted in FIG. 46;

FIGS. 48A-48D schematically depict views of an illustrative configuration of a channel for use in a device for cutting soft tissue;

FIG. 49 schematically depicts a side view of an illustrative configuration of a device for cutting soft tissue;

FIG. 50 schematically depicts a material for an illustrative configuration of a blade shaft of the device device for cutting soft tissue depicted in FIG. 49;

FIG. 51 schematically depicts an exploded view of an illustrative configuration of the device for cutting soft tissue depicted in FIG. 49;

FIG. 52 schematically depicts an exploded view of an illustrative configuration the device for cutting soft tissue;

FIG. 53 schematically depicts a cross-section view of the distal portion of the housing depicted in FIG. 52, taken at box 198 and with expanders added-in;

FIG. 54 schematically depicts an illustrative configuration of components of the device for cutting soft tissue;

FIG. 55 schematically depicts a side view of an illustrative configuration of components of the device for cutting soft tissue;

FIG. 56 schematically depicts a side view of an illustrative configuration of components of the device for cutting soft tissue;

FIG. 57 schematically depicts a top view of an illustrative configuration of a component of the device for cutting soft tissue;

FIG. 58 schematically depicts a perspective view of an illustrative configuration of components of the device for cutting soft tissue;

FIG. 59 schematically depicts a perspective view of an illustrative configuration of components of the device for cutting soft tissue;

FIGS. 60A and 60 schematically depict side views of an illustrative adjustment of the blade between a protected position and a cutting position;

FIGS. 61A and 61B schematically depict side views of an illustrative adjustment of the blade between a protected position and a cutting position;

FIG. 62 schematically depicts a perspective view of an illustrative configuration of the device for cutting soft tissue;

FIGS. 63A-63D schematically depict views of an illustrative configuration of components for use with or in the device for cutting soft tissue;

FIG. 64 schematically depicts a side view of an illustrative configuration of components for the device for cutting soft tissue;

FIG. 65 schematically depicts a side view of an illustrative configuration of components for the device for cutting soft tissue;

FIG. 66 schematically depicts a perspective view of an illustrative configuration of the device for cutting soft tissue;

FIGS. 67A-67C schematically depict views of an illustrative configuration of components for use with or in the device for cutting soft tissue;

FIG. 68 schematically depicts a perspective view of an illustrative configuration of the device for cutting soft tissue;

FIG. 69A schematically depicts a perspective view of an illustrative configuration of the device for cutting soft tissue;

FIG. 69B schematically depicts a cross-section view of the device for cutting soft tissue depicted in FIG. 69A, taken along rectangle 69B;

FIG. 70 schematically depicts a side view of an illustrative configuration of the device for cutting soft tissue;

FIG. 71A schematically depicts a side view of a distal end of an illustrative configuration of the device for cutting soft tissue;

FIG. 71B schematically depicts a cross-section view of the device for cutting soft tissue depicted in FIG. 71A, taken along rectangle 71B;

FIG. 72 schematically depicts a side view of an illustrative configuration of the device for cutting soft tissue, where the device is positioned within a patient;

FIGS. 73A-73C schematically depict views of illustrative configurations of components of the device for cutting soft tissue;

FIGS. 74A and 74B schematically depict views of an illustrative configuration of the device for cutting soft tissue;

FIGS. 75A and 75B schematically depict views of illustrative configurations of components of the device for cutting soft tissue;

FIGS. 76A-76C schematically depict views of illustrative positions of components of the device for cutting soft tissue;

FIGS. 77A-77C schematically depict views of illustrative positions of components of the device for cutting soft tissue, with the device for cutting soft tissue positioned within a patient;

FIGS. 78A-78H schematically depict views of an illustrative procedure using a device for cutting soft tissue;

FIGS. 79A-79D schematically depict views of an illustrative technique for confirming soft tissue has been cut; and

FIGS. 80 schematically depicts an exploded view of a distal end of an illustrative device for cutting soft tissue;

FIGS. 81A and 81B schematically depict an illustrative adjustment of a blade between an exposed or cutting position and a protected position;

FIG. 82 schematically depicts a sectional view of a distal end of an illustrative device for cutting soft tissue;

FIG. 83 schematically depicts a side view of an illustrative blade holder and blade; and

FIGS. 84A and 84B schematically depict an illustrative adjustment of a blade between an exposed or cutting position and a protected position.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” even if not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

References in the specification to “a configuration”, “some configurations”, “other configurations”, etc., indicate that the configuration described may include one or more features, structures, and/or characteristics. Such recitations, however, do not necessarily mean that all embodiments include those features, structures, and/or characteristics. Additionally, when features, structures, and/or characteristics are described in connection with one configuration, such features, structures, and/or characteristics may also be used in connection with other configurations even if not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Illustrative devices for cutting soft tissue may include a blade for cutting soft tissues and two inflatable balloons on either side of the shaft of the device to keep non-target tissues (such as nerves and blood vessels) away from the blade while it is active. Balloon inflation may be achieved via an inflation bellows located in the handle of the device. The blade may move along a ramp mechanism in the shaft of the device to transition from an inactive (or “safe”) position inside the shaft to an active position for cutting and back to an inactive position. Such devices for cutting soft tissue (e.g., in carpal tunnel release procedures, etc.) work well in practice. Moreover, minimally invasive carpal tunnel release procedures have been successfully performed using such devices.

Medical devices with improved features to make the devices easier to use and/or more effective for minimally invasive soft tissue cutting procedures may result in improved patient outcomes. For example, improvements to the blade, movement of the blade, the protective guards or expanders (e.g., balloons), expansion of the expanders (e.g., inflation of the balloons), functioning of the handle of the device, and/or visualization or sensing with the device may be desirable and improve the patient experience. At least some of these objectives are discussed herein. Further, while the illustrated configurations show an example configuration of a soft tissue cutting device, such as a transverse carpal ligament cutting device, many different types of devices for the same or different procedures can be shipped, primed, and tested prior to use, according to systems and methods disclosed herein. As but one set of examples, soft tissue cutting devices described herein and/or variations thereof may be used to cut other ligaments in the hand, wrist, or other parts of the body, such as but not limited to the elbow, shoulder, ankle, or other joints in the body. Thus, although much of the following description focuses on use of the described device for cutting the transverse carpal ligament in a carpal tunnel release procedure, the devices, systems, and methods described herein should not be interpreted as being limited to.

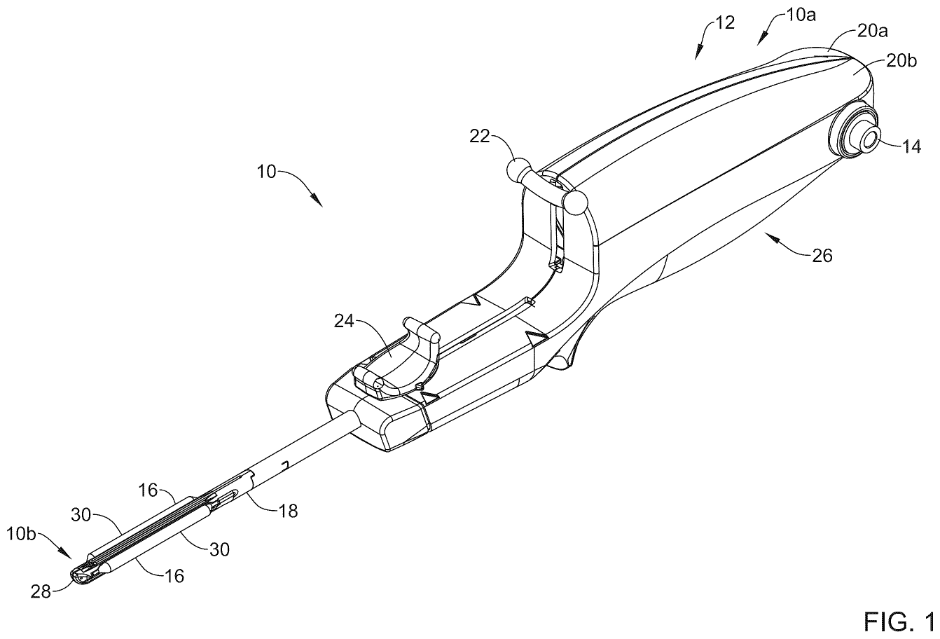

FIG. 1 is a perspective view of device 10 for cutting body tissue (e.g., a soft tissue cutting device), such as one or more ligaments of a patient (e.g., a transverse carpal tunnel ligament and/or other suitable soft tissues). The device 10 may have a proximal end 10a and a distal end 10b. The proximal end 10a of the device 10 may include a handle assembly or handle 12 and a priming port 14, among other components. The distal end 10b of the device 10 may include one or more guards or expanders 16 (e.g., shown in an unexpanded configuration in FIG. 1), a shaft 18, and a blade (not shown in FIG. 1), among other components. As depicted in FIG. 1, the shaft 18 may extend distally from the handle 12.

The handle 12 may include a housing 20, a first actuator 22 in communication with the expander(s) 16, and a second actuator 24 in communication with the blade. In some configurations and as depicted in FIG. 1, the first actuator 22 may be or may include a lever, and the second actuator 24 may be or may include a slider. The handle 12 may include additional and/or alternative components, as desired.

The housing 20 may have any suitable configuration. In some examples, the housing 20 may be formed from one or more housing components. When the housing 20 is formed from two or more housing components, the housing components may be coupled together using any suitable connectors or connection technique, including, but not limited to, snap connections, threaded connectors, adhesives, and/or other suitable connectors or connection techniques. In some examples, the housing 20 may be formed from a first portion 20a and a second portion 20b coupled together along a longitudinally extending seam using snap connections and/or adhesives, but other suitable configurations are contemplated.

The housing 20 may have a configuration that facilitates a user gripping and using the device 10 with a single hand. For example, the housing 20 may include a grip portion 26 configured to facilitate a user adjusting the first actuator 22 with a thumb of the single hand gripping the handle 12 and adjusting the second actuator 24 in a proximal/distal direction with the thumb used to actuate the first actuator 22. Other suitable configurations are contemplated.

At least a portion of the distal end 10b of the device 10 may be configured for insertion into a patient. For example, the shaft 18 may have a tip 28 (e.g., a probe tip and/or other suitable tip) that may be rigid and blunt (e.g., smooth-edged, rounded, etc.)

to facilitate insertion into a target area of the patient (e.g., a carpal tunnel region and/or other suitable region of the patient). Further, the shaft 18 may be echogenic and/or include diagnostic equipment to facilitate visualization the shaft 18 within the patient using ultrasound or other suitable imaging techniques and/or to facilitate visualization of a region (e.g., the carpal tunnel region and/or other suitable region) of the patient during insertion or surgery.

Although not depicted, an axial depth indicator may be positioned along the shaft 18 distal of the handle 12 and proximal of the expanders 16. In some configurations, the axial depth indicator may be or may include a spring-loaded pin that is slidable along the shaft 18 by pressing ends of the axial depth indicator toward each other. By sliding the axial depth indicator along the shaft 18 as the shaft 18 is inserted into the patient, the axial depth indicator may be used to indicate the depth of insertion of the shaft 18 or distal end 10b of the device 10 into the patient.

The distal end 10b of the device 10 may include the blade and the blade may be configured to travel along a blade travel segment of the shaft 18. For example, the blade may initially be covered or protected by the shaft 18 (e.g., the blade may be in an inactive state or position), as depicted in FIG. 1. The blade, which may include or may be coupled with a blade shaft coupled with the second actuator 24, may move proximally and be exposed from the shaft 18 (e.g., the blade moves into an active state or position) as the second actuator 24 is adjusted proximally. As the second actuator 24 approaches and/or reaches a full travel distance, the blade may return to a position at which the blade may be covered or protected by the shaft 18.

The one or more expanders 16 may extend along the shaft 18. In some examples, the one or more expanders 16 may extend along (e.g., longitudinally along) at least a portion of the shaft 18 at which the blade is exposed (e.g., such as the blade travel segment of the shaft 18). In some examples, the one or more expanders 16 may extend along an entire distance of travel of the exposed blade. Once the expanders 16 are expanded, the blade may be actuated and exposed for cutting soft tissue of the patient.

Any suitable number of expanders 16 may be used. In some examples, two expanders may be used and configured to expand laterally and/or radially outward to move tissue away from a target issue to be cut by the blade.

The expanders 16 may be any suitable type of expander including, but not limited to, a balloon, an elastic material, a laterally adjustable component, a longitudinally adjustable component, and/or other suitable type of expander. In some examples, one or more of the expanders 16 may be a balloon 30, as depicted in FIG. 1. When inflated, the balloons 30 may expand radially outwardly from the shaft 18, may be elongate along a longitudinal axis of the shaft 18, and may have a spherical, oval, bilobular, and/or other suitable cross-sectional configuration. When deflated, the balloons 30, may be flush with the shaft 18 and/or have a reduced diameter relative to when inflated to facilitate insertion of the shaft 18 into a patient to a target site. When one or more balloons 30 are used, the balloons 30 may create a “safe zone” within a patient (e.g., within a patient's carpal tunnel region) at which the blade may be moved to an active or exposed position.

At least a portion of the balloon 30 may be configured to inflate and deflate. In some configurations, the entire balloon 30 is expandable and may inflate and deflate. In other configurations, only part of the balloon 30 is expandable. For example, a balloon 30 with only a portion that is expandable may have a fixed portion and an expandable portion.

While two lateral balloons 30 are depicted in FIG. 1, any number of radially expanding balloons 30 may be used and may be placed anywhere about the shaft 18 to create and/or expand one or more safe zones and/or secure the distal end 10b of the device 10 in a desired position within the patient. In one example of utilizing the balloons 30 within a carpal tunnel region of a patient, as the radially expanding balloons 30 inflate, the balloons 30 may move the flexor tendons, median nerve, and/or ulnar neurovascular bundle away from the device 10 to help create a region in which the transverse carpal ligament can be cut while preventing nearby at-risk structures from being cut, effectively increasing the safe zone.

The balloons 30 may inflate to any suitable size to accommodate a size of a target region of a patient (e.g., a wrist, a carpal tunnel region, etc.). For example, in some cases, the balloons 30 may be provided with a specific size, such that when the balloons 30 are fully inflated, the balloons may have a specific inflated size. In patients with larger wrists, larger balloons may be used and in patients with smaller wrists, smaller balloons may be used. In one configuration, each of two or more balloons 30 may inflate to a similar or same diameter. In another configuration, one of the two or more balloons 30 may inflate to one size and another balloon 30 may inflate to a different size. In other cases, the balloons 30 may have a standard size but may be partially inflated or fully inflated to have a variety of different inflated sizes. In some examples, the balloon inflation may be graded to allow the operator to choose a particular balloon diameter. In certain instances, the inflation of the balloon 30 may be pressure dependent, such that the balloon 30 manually or automatically inflates until a specific pressure is exerted on the balloon surface.

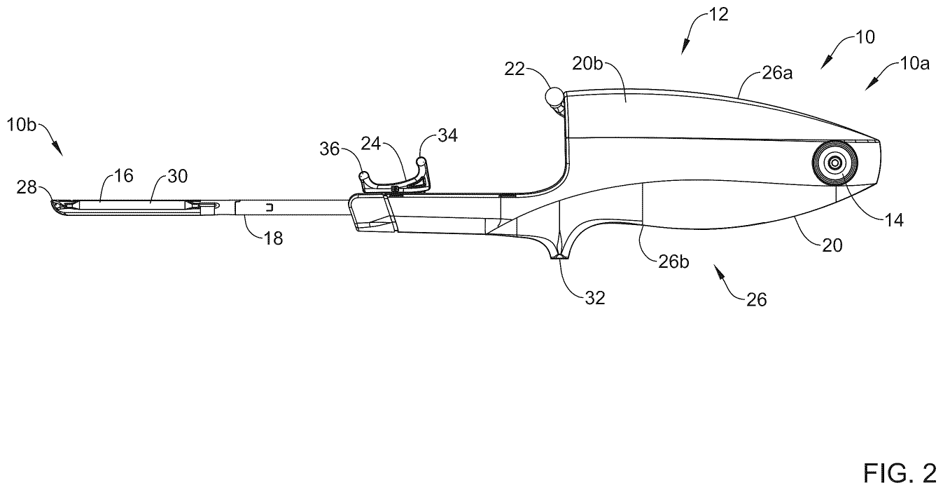

FIG. 2 depicts a side view of the device 10. As depicted in FIG. 2, the first actuator 22 is in an unactuated position such that the expanders 16 are not expanded and the second actuator 24 is in a locked configuration with the blade distally positioned and unexposed (e.g., with the blade in an inactive position).

As discussed, the housing 20 of the handle 12 may include the grip portion 26. In some examples, a top surface 26a of the grip portion 26 may be configured to receive a palm of a hand of a user and a bottom surface 26b of the grip portion 26 may be configured to receive one or more fingers (e.g., a middle finger, a ring finger, and a little or pinky finger) of the user. Further, the grip portion 26 may include a protrusion 32, where the protrusion 32 may be positioned along the handle 12 such that a user may position an index finger of the hand gripping the handle 12 distal of the protrusion 32 and a middle finger proximal of the protrusion 32. When a user grips the handle 12 with a palm of a hand on the top surface 26a of the grip portion 26, with a middle finger, a ring finger, and a little finger on the bottom surface 26b of the grip portion 26 and proximal of the protrusion 32, and with an index finger distal of the protrusion 32, the thumb on the hand of the user that is gripping the handle 12 may be positioned to adjust (e.g., actuate) the first actuator 22 and adjust (e.g., longitudinally adjust) the second actuator 24. Other suitable configurations of the grip portion 26 of the handle 12 are contemplated.

The second actuator 24 may have any suitable shape configured to be longitudinally adjusted along the handle 12 and/or relative to the shaft 18. In one example, the second actuator 24 may be configured to be engaged with a thumb of a hand of a user that is grasping the handle 12, for example as discussed. In some examples, the second actuator 24 may have one or more extensions or protrusions that the thumb or other suitable digit of the user may engage to adjust the second actuator 24 and a blade coupled therewith. As depicted in FIG. 2, the second actuator 24 may include a first extension 34 (e.g., a proximal extension) and a second extension 36 (e.g., a distal extension) spaced distal of the first extension 34, but other suitable configurations are contemplated including, but not limited to, second actuators 24 with more than two extensions or second actuators 24 omitting one or both of the extensions. After a user has actuated the first actuator 22 to unlock the second actuator 24 and expand the expanders 16, a user may engage the second actuator 24 at a location of one or both of the extensions 34, 36 and/or between the extensions 34, 36 (e.g., in a recessed area between the extensions 34, 36) and a force may be applied to second actuator 24 in the proximal direction to move the second actuator 24 and the blade (not shown) in communication with the second actuator 24 in the proximal direction. Once adjusted proximally, the second actuator 24 may be adjusted distally, as desired, to distally move the blade.

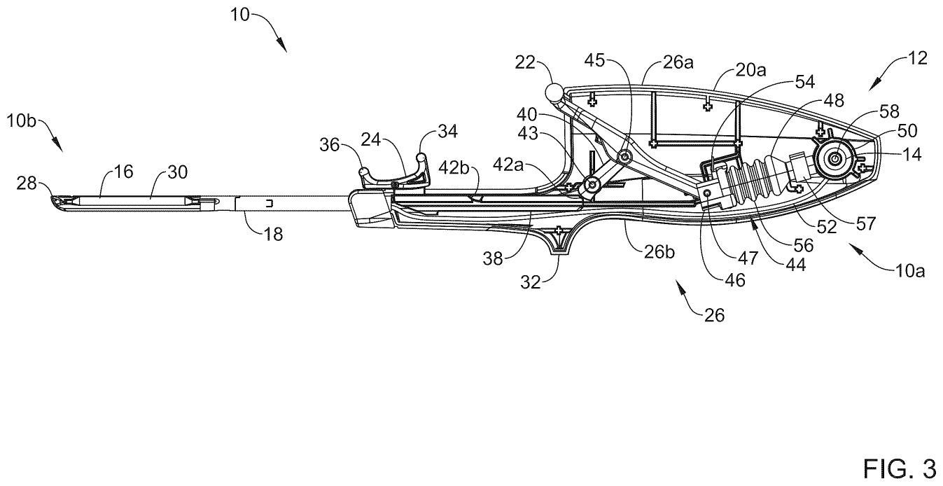

FIG. 3 schematically depicts the side view of the illustrative device 10 depicted in FIG. 2, but with the second portion 20b of the housing 20 removed to show an interior of the handle 12. As depicted in FIG. 3, the first actuator 22 may be in selective communication with the second actuator 24. Further, the device 10 may include an expansion system 44 coupled with the first actuator 22 and in communication with (e.g., in fluid communication with) the priming port 14 and the expanders 16 (e.g., the balloons 30).

The first actuator 22 may be in selective communication with the second actuator 24 in any suitable manner to adjust the second actuator 24 between a locked configuration and an unlocked configuration. For example, the first actuator 22 may be in direction communication with the second actuator 24 or in indirect communication with the second actuator 24 through one or more components. In some examples, the first actuator 22 may be in selective communication with the second actuator 24 through an elongate component 38 (e.g., an elongate extension or other suitable component) extending proximally from the first actuator 22 and/or one or more linkages 40 extending from the first actuator 22.

The elongate component 38 may be configured to adjust with the second actuator 24 as the second actuator 24 adjusts longitudinally along the handle 12. The elongate component 38 may be monolithically formed with the second actuator 24 and/or coupled to or with the second actuator 24 in any suitable manner. Further, the elongate component 38 may be formed from a single component or two or more components coupled to one another.

The elongate component 38 my have any suitable configuration designed to be in selective communication with the first actuator 22 to adjust the second actuator 24 between a locked configuration and an unlocked configuration. In some examples, the elongate component 38 may include one or more engagement portions configured to receive the linkage(s) 40 to releasably fix the second actuator 24 in a locked configuration at a desired longitudinal position along the handle 12. In one example, the elongate component 38 may include a first engagement portion 42a (e.g., a proximal engagement portion) and a second engagement portion 42b (e.g., a distal engagement portion) spaced distally from the first engagement portion 42a. The engagement portions 42a, 42b may be indentations or other suitable engagement portions in the elongate component 38 and the space between the engagement portions 42a, 42b may be raised relative to the engagement portions 42a, 42b. In some examples, the first engagement portion 42a may be associated with a protected or inactive first position of the blade and the second engagement portion 42b may be associated with a protected or inactive second position of the blade. Other suitable configurations of the elongate component 38 are contemplated.

The one or more linkages 40 may be configured to adjust with the first actuator 22 to selectively engage the elongate component 38 (e.g., to selectively engage the engagement portions 42a, 42b) to limit movement of the second actuator 24. The one or more linkages 40 may include a single linkage or two or more linkages coupled together.

In some examples, a single linkage 40 may be used, where the single linkage 40 may have a first end pivotably coupled with the first actuator 22 and a second end configured to engage the elongate component 38. The single linkage 40 may be further pivotably coupled with the housing 20 or other structure at a location between the end of the single linkage coupled with the first actuator 22 and the end of the single linkage 40 configured to engage the elongate component 38. In some examples, the pivotable coupling between the linkage 40 and the housing 20 or other suitable structure may have a fixed pivot point and the pivotable coupling between the linkage 40 and the first actuator 22 may have an adjustable pivot point that adjusts rotationally relative to the fixed pivot point between the linkage 40 and the housing 20. In some examples, the fixed pivot point may be an axis of a fixed pivot pin 43 secured in a wall of the housing 20 and extending through at least one of the one or more linkages 40. In some examples, the adjustable pivot point may be an axis of a pivot pin 45 extending through at least one of the one or more linkages 40 and the first actuator 22. Other suitable configurations of the one or more linkages 40 are contemplated.

The coupling between the one or more linkages 40 and the first actuator 22 may be any suitable type of coupling. In some examples, the coupling between the one or more linkages 40 and the first actuator 22 may be or may include an over-center mechanism. When the expansion system 44 is biased to an expanded configuration and/or the first actuator 22 is biased to an unactuated position (e.g., at which the expanders 16 are not expanded or are contracted), the over-center mechanism coupling between the one or more linkages 40 and the first actuator 22 may facilitate the first actuator 22 maintaining its actuated position after the first actuator 22 has been adjusted to the fully actuated position and the forces applied to the first actuator 22 are removed until a further force is applied to the first actuator 22 to overcome the over-center mechanism and allow the bias on the first actuator 22 and/or other forces to adjust the first actuator 22 to an unactuated position.

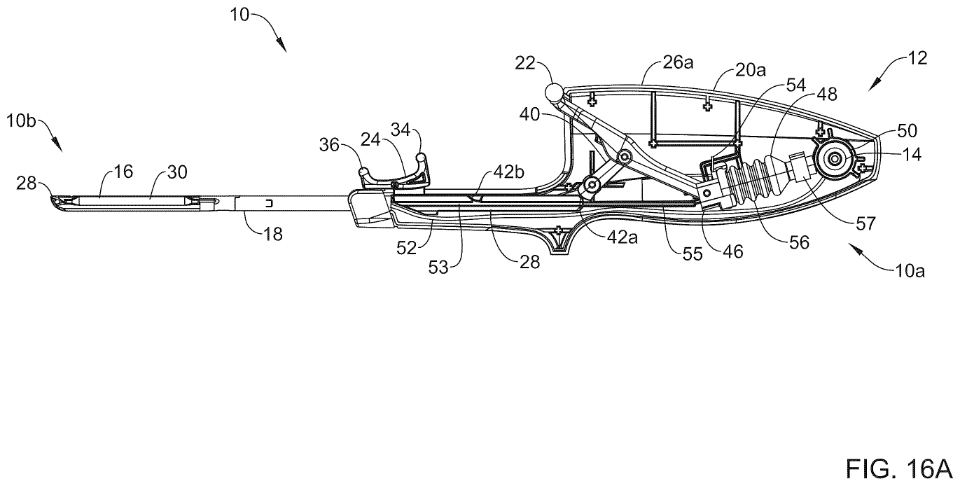

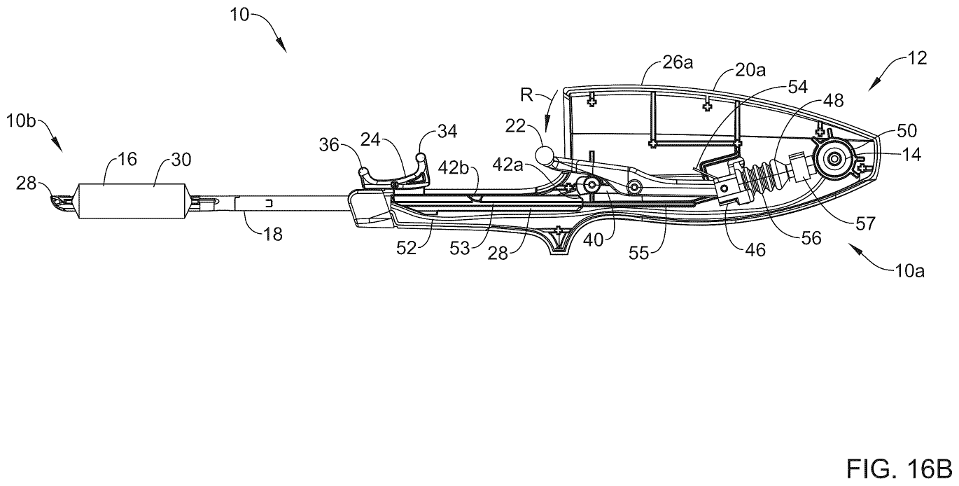

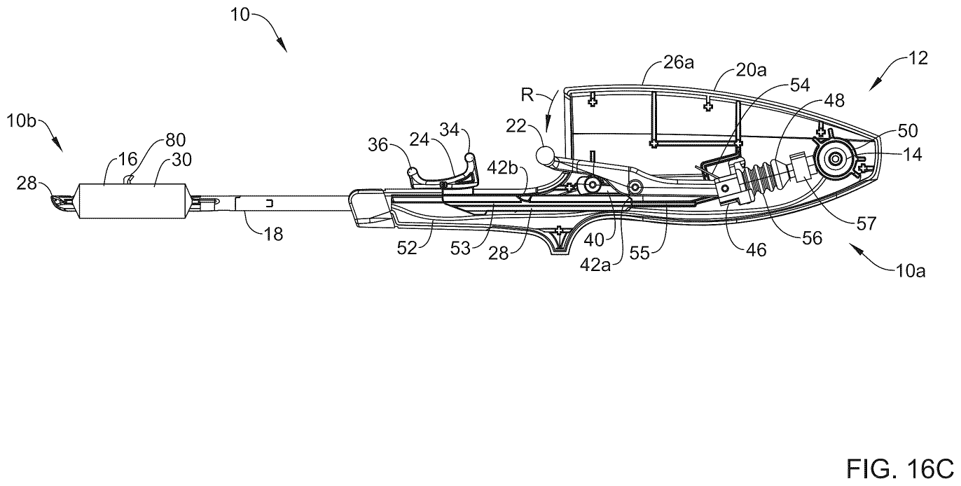

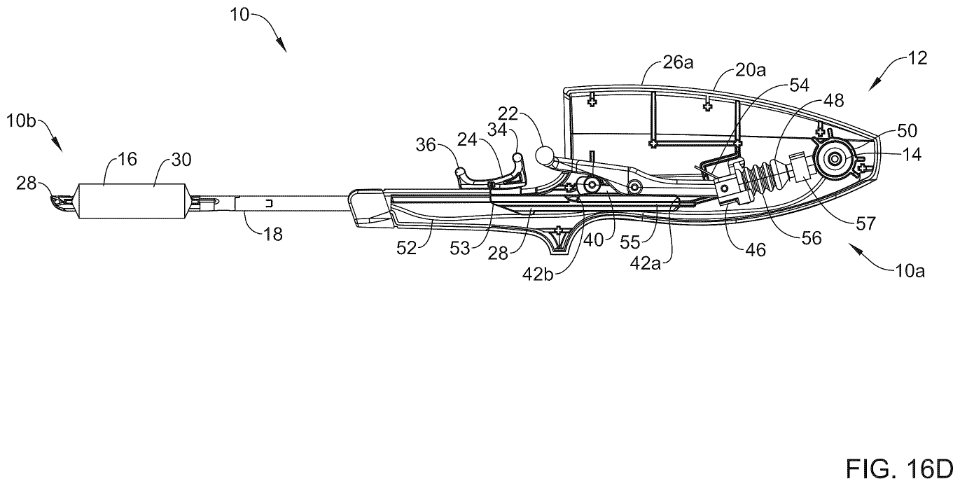

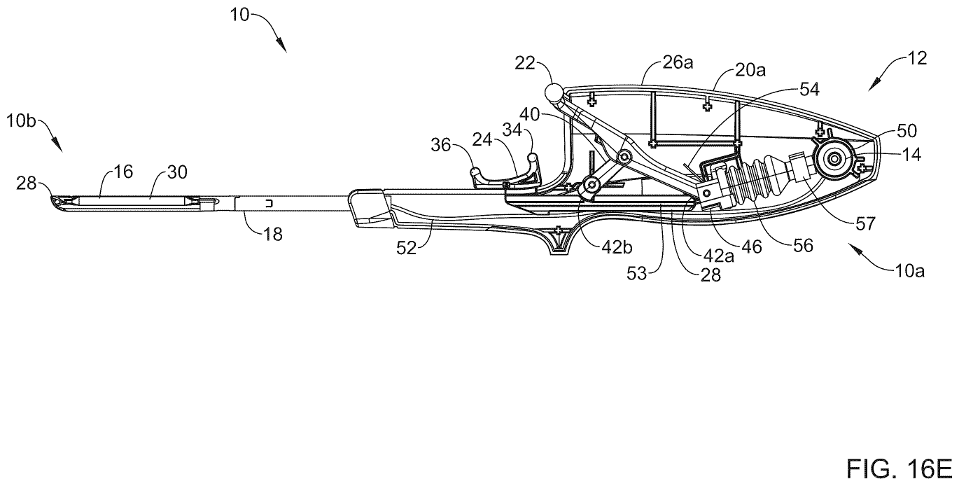

In operation, the one or more linkages 40 may be configured to engage the first engagement portion 42a of the elongate component 38 when the second actuator 24 is in a first locked configuration and the blade is in a protected first position. After the first actuator 22 is actuated to expand the expanders 16, the one or more linkages 40 may disengage the first engagement portion 42a such that the second actuator 24 is in the unlocked configuration. When in the unlocked configuration, the second actuator 24 may be longitudinally adjusted (e.g., in a proximal direction) to longitudinally adjust the blade to an exposed or cutting second position (not shown in FIG. 3) until the blade moves to a proximal, protected third position and the second engagement portion 42b is proximate the one or more linkages 40. Such movements are depicted in FIGS. 16A-16E. The raised portion between the engagement portions 42a, 42b, when included, may be configured to engage the one or more linkages 40 and prevent first actuator 22 from moving to an unactuated position (e.g., a fully unactuated position) and contracting the expanders 16 (e.g., deflating the balloons 30) while the blade is in the exposed or cutting second position (e.g., while the blade is exposed between the protected first position and the protected third position).

The expansion system 44 may be any suitable system configured to facilitate expanding and/or contracting the expanders 16 in response to actuation or adjustment of the first actuator 22. In some configurations, the expansion system 44 may include a connector 46, an expansion device 48 (e.g., a compressor and/or other suitable expansion device), and a manifold 50. Further, the expansion system 44 may include one or more tubes 52 configured to move fluid from the manifold 50 to the expanders 16.

The connector 46 may be any suitable type of connector for coupling the first actuator 22 with the expansion device 48. In one example, the connector 46 may be pivotably coupled with an end of the first actuator 22 such that the first actuator 22 pivots about an adjustable (e.g., a longitudinally adjustable) pivot point at the connector 46. In some examples, the adjustable pivot point at the connector 46 may be an axis of a pivot pin 47 extending through the first actuator 22 and the connector 46. In some examples, a first end of the first actuator 22 may be configured for a user to engage, a second end of the first actuator 22 may couple with the connector 46, and the first actuator 22 may couple with the one or more linkages 40 at a location between the first end and the second end.

In some configurations, the pivot pins 43, 45, 47 may be omitted. When omitted, the pivot pins 43, 45, 47 may be replaced with one or more bosses or protrusions configured to facilitate pivoting of the components at the bosses. Other suitable configurations are contemplated.

In some configurations, one or more springs 54 may engage the connector 46, the expansion device 48, and/or the second end of the first actuator 22 to bias the first actuator 22 to an unactuated position and/or the expansion device 48 to an expanded position. The one or more springs 54, when included, may be any suitable type of spring(s). In some examples and as depicted in the Figures, the one or more springs 54 may be or may include a torsional spring configured to engage the first actuator 22 and the connector 46. Alternatively or additionally, the one or more springs 54 may be or may include a return spring extending along an exterior surface of and coaxial with the expansion device 48 to bias the expansion device 48 to an expanded configuration. When the return spring positioned around the expansion device 48 is used, the expansion device 48 may include a configuration (e.g., pleats, an overmold, etc.) configured to receive or engage the return spring (e.g., the return spring may be added to expansion device 48 prior to coupling with the manifold 50, the return spring may be threaded on pleats of the expansion device 48, etc.), but other suitable configurations are contemplated. In some examples, the one or more springs 54 may be or may include the material of the expansion device 48, which may be configured to bias the expansion device 48 to an expanded configuration. Other suitable spring or biasing configurations are contemplated.

As discussed, the expansion device 48 may be coupled with the first actuator 22 and in response to actuation of the first actuator 22, the expansion device 48 may adjust to expand the expanders 16. When the expanders 16 are or include balloons 30 and/or other suitable inflatable devices, the expansion device 48 may be in fluid communication with the manifold 50, which may be in fluid communication with the balloons 30 and/or other suitable inflatable devices via one or more tubes 52. In one example, the first actuator 22 may be fully actuated such that the expansion device 48 contracts and causes fluid to pass through the tubes 52 to the balloons 30 and the one or more linkages 40 are in an over-center position to maintain the first actuator 22 in the fully actuated position with the balloons 30 expanded, but other suitable configurations are contemplated.

The expansion device 48 may have any suitable configuration and/or components configured to facilitate expanding and/or contracting the expanders 16 in response to forces acting on the first actuator 22 to adjust the first actuator 22 between a fully actuated position (e.g., in which the expanders 16 are in an expanded position) and an unactuated position (e.g., in which the expanders 16 are in the contracted position). For example, the expansion device 48 may include, but is not limited to, one or more of one or more compressors, one or more cylinders, one or more plungers, one or more syringes, one or more bellows, a set of one or more mechanical linkages, and/or other suitable components configured to expand and/or contract the expanders 16. In some examples, the expansion device 48 may be or may include a compressor or bellows 56 configured to expand and/or contract to move fluid (e.g., air, liquid, foam, etc.) between the bellows 56, the manifold 50, the tubes 52, the expanders 16 (e.g., the balloons 30, etc.), and/or other suitable components of the device 10. In some examples, the expansion device 48 may a plunger (not shown) in communication with the bellows 56.

The bellows 56, when included, may have any suitable configuration. In some examples, the bellows 56 may include pleats, as depicted in FIG. 3, configured to facilitate contracting and expanding the bellows 56 as the first actuator 22 is adjusted and/or as forces are otherwise acting on the bellows 56 (e.g., during priming, as discussed herein or otherwise). As discussed, the bellows 56 may be biased to an expanded configuration in response to one or more springs acting on the bellows 56 and/or one or more springs or spring forces of the bellows 56.

The manifold 50 may have any suitable shape and/or configuration designed to hold fluid received via the priming port 14 and facilitate expanding the expanders 16 with the fluid received. In some examples, the manifold 50 may be in fluid communication with the priming port 14, the expansion device 48, the tubes 52, the expanders 16, and/or other suitable components of the device 10 in any suitable manner. In some configurations, the manifold 50 may be omitted or incorporated into one or more other components of the device 10.

The manifold 50, when included, may be coupled, formed with, or otherwise in fluid communication the priming port 14, the expansion device 48, the tubes 52, the expanders 16, and/or other suitable components of the device 10 in any suitable manner. For example, the manifold 50 may be formed integral with, formed monolithically with, press fit with, crimped to, connected with adhesive to, press fit with, and/or coupled with the priming port 14, the expansion device 48, the tubes 52, the expanders 16, and/or other suitable components of the device 10. In one example, the manifold 50 may be coupled with the expansion device 48 using a clamp and/or crimp 57 around the manifold 50 and the expansion device 48. In another example, an end of the tubes 52 may be coupled with the manifold 50 using a press fit technique and/or adhesive. In another example, the manifold 50 may be monolithically formed with the priming port 14. Other suitable techniques for coupling the manifold 50 with components of the device 10 are contemplated.

The priming port 14 may have any suitable configuration configured to receive a fluid transfer device (e.g., a priming device or other suitable fluid source). Example fluid transfer devices include, but are not limited to, syringes, flexible reservoirs, and/or other suitable fluid sources.

In some configurations, the priming port 14 may include one or more valves 58. In some examples, the valve(s) 58 may be one-way valves or check valves configured to facilitate receiving fluid from the fluid transfer device received at the priming port and blocking fluid from exiting the valves when the fluid transfer device is not received at the priming port 14.

The priming port 14 may be configured to receive and/or couple with a fluid transfer device in any suitable manner. For example, the priming port 14 may be configured to receive and/or couple with the fluid transfer device using one or more coupling techniques including, but not limited to, luer lock connections, friction or pressure fit connections, threaded connections, bayonet connections, ball-detent connections, and/or other suitable types of coupling techniques. In some examples, the priming port 14 may be configured to couple with the fluid transfer device using a luer lock connection in a manner that opens the valve 58 of the priming port 14.

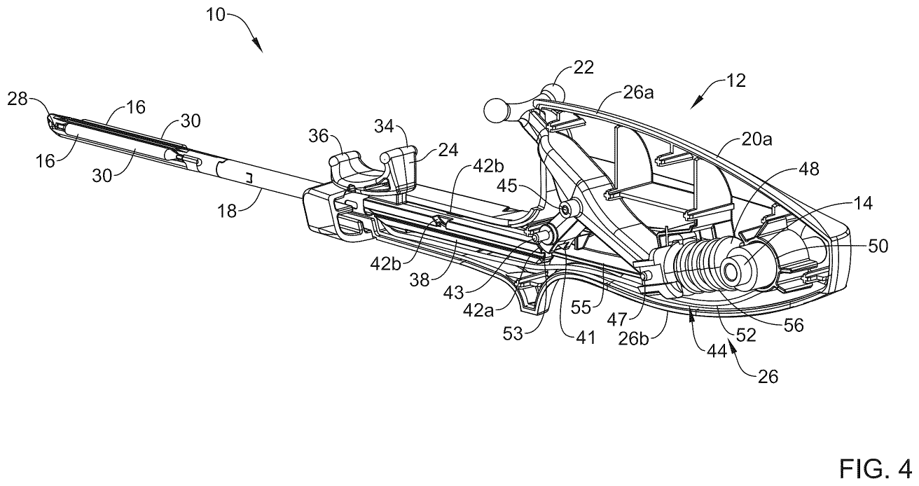

FIG. 4 depicts a schematic back-side perspective view of the illustrative device 10 depicted in FIG. 3 with the second portion 20b of the housing 20 removed. As depicted in FIG. 4, the elongate component 38 may include alignment features such as channels 53 and ledges 55, where one or more channels 53 of the elongate component 38 may be configured to engage one or more ledges 55 of the housing 20. In some examples, the elongate component 38 may include two elongate channels 53, where each elongate channel 53 may be configured to engage a ledge 55 of each of the first portion 20a and the second portion 20b of the housing 20. Alternatively or additionally, the elongate component 38 may include one or more ledges configured to engage one or more channels of the housing 20. When included, the channels 53 and/or ledges 55 may facilitate linear movement of the elongate component 38 in response to movement of the second actuator 24. Other suitable features for ensuring a desired alignment of the elongate component 38 may be used and/or alignment features may be omitted.

Further, the linkage 40 may include one or more feet 41 configured to engage the engagement portions 42a, 42b. In some examples and as depicted in FIG. 4, the linkage 40 may include two feet 41 and may be configured to engage separate slots of the engagement portions 42a, 42b (for example, two slots of the second engagement portions 42b are depicted in FIG. 4).

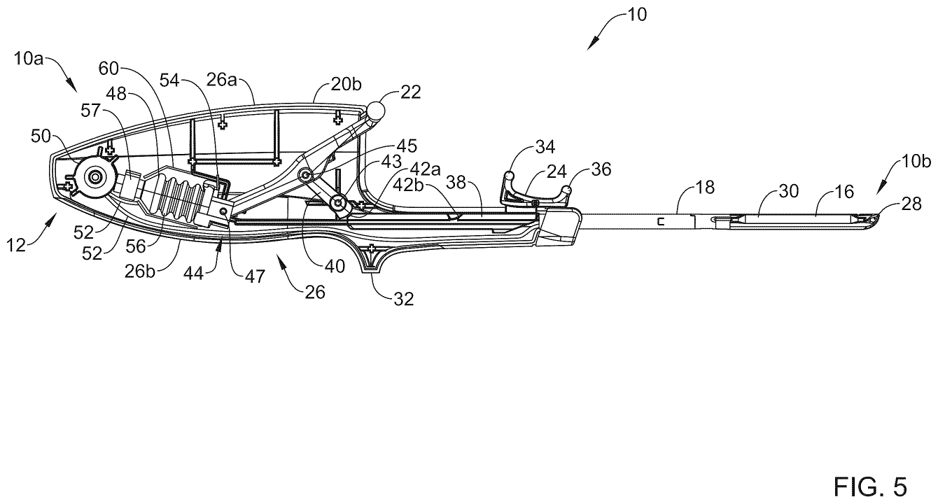

FIG. 5 depicts a schematic side view of the illustrative device 10 depicted in FIG. 1, where the depicted side is opposite a side depicted in FIGS. 2 and 3 and the second housing portion 20b has been removed to schematically depict an interior of the handle 12. Several components of the handle 12 depicted in FIG. 5 were discussed with respect to FIG. 3 and are not re-discussed here.

The one or more tubes 52 may extend from the manifold 50 to the expanders 16. When two balloons 30 or other inflatable devices are used as the expanders 16, two tubes 52 may extend from the manifold 50, where each tube 52 may extend from the manifold to a different one of the two balloons 30. Other suitable number of tubes 52 may be used with one or more balloons 30 and/or other suitable expanders 16.

The tubes 52 may have any suitable size and/or configuration. In some examples, the tubes 52 may have an inner diameter that facilitates utilizing capillary action to move liquid fluid from the expansion system 44 to the expanders 16 without drawing air into the expanders 16. Preventing air from entering the expanders 16 may facilitate identifying the expanders 16 in images (e.g., ultrasound images or other suitable images) of the device 10 within a patient as non-air fluid is more readily viewable on images than expanders 16 with air therein. Further, as air is a compressible fluid, preventing air from entering the expanders 16 may facilitate exerting a predictable pressure on tissue in the patient at locations proximate the expanders 16.

FIG. 5 depicts a carrier 60 configured to facilitate maintaining a position of the expansion device 48 within the housing 20 of the handle 12. The carrier 60 may extend around one or more edges or sides of the expansion device 48 to carry or stabilize the expansion device within the handle 12. Although not required, the connector 46 may engage and/or otherwise couple with the carrier 60 to stabilize the expansion device within the housing 20, while allowing the expansion device 48 to contract and/or expand in response to adjustments or actuation of the first actuator 22 for expanding and/or contracting the expanders 16.

As discussed, the blade may be in communication with and/or coupled with the second actuator 24. As depicted in FIG. 5, a blade shaft 62 of or from which the blade extends may connect with the elongate component 38 of or coupled with the second actuator 24.

The blade 80 and the blade shaft 62 may be referred to collectively as the blade. In some configurations, the blade and the blade shaft 62 may be monolithically formed. Alternatively, the blade and blade shaft 62 may be separate components coupled together using one or more suitable coupling techniques including, but not limited to, an over mold technique, a press or friction fit technique, an adhesive technique, a snap technique, a luer lock technique, a threaded coupling technique, and/or other suitable coupling techniques.

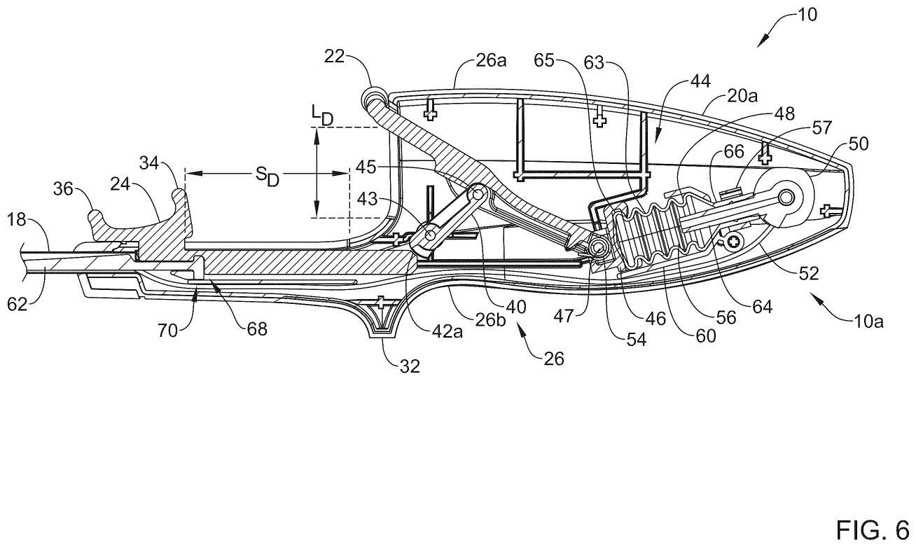

FIG. 6 depicts a schematic cross-section view of the handle 12 of the device 10, taken along a vertical longitudinally extending plane through a central axis of the device 10. As depicted in FIG. 6, the second actuator 24 may be configured to adjust or move a horizontal distance SD and the first actuator 22 may be configured to adjust or move a vertical distance LD. Other suitable configurations are contemplated.

As depicted FIG. 6, the expansion device 48 may be coupled with the connector 46 and positioned within the carrier 60. In some examples, the connector 46 may be configured to couple with the expansion device 48 by engaging a valley 63 between two peaks 65 of the expansion device 48. Further, the connector 46 may receive the first actuator 22 and the spring 54, where a pivot pin 47 may couple the first actuator 22 with the connector 46 and the spring 54 may bias the first actuator 22 to an unactuated position and the expansion device 48 to an expanded position. Other suitable configurations of the first actuator 22, the connector 46, and the expansion device 48 are contemplated.

The expansion device 48 may be coupled with the manifold 50 via the clamp and/or crimp 57, as discussed. In some examples and as depicted in FIG. 6, the manifold 50 may have an elongate portion 64 configured to be inserted into the expansion device 48 through a connector portion 66. The clamp or crimp 57 may be clamped or crimped over the connector portion 66 of the expansion device 48 at a location overlapping the elongate portion 64 of the manifold 50 to secure the manifold 50 with the expansion device 48. In some examples, the connection between the expansion device 48 and the manifold 50 may be a fluid tight (e.g., hermetic) connection.

As depicted in FIG. 6, the elongate component 38 and the second actuator 24 may be monolithically formed. Other suitable configurations, however, are contemplated.

The second actuator 24 and/or the elongate component 38 may include a connector portion 68, where the connector portion 68 may be configured to engage a connector end 70 of the blade or the blade shaft 62. In some examples, the connector portion 68 may have a shape designed to mate with the connector end 70 of the blade or blade shaft 62. When so configured, the blade or blade shaft 62 and the second actuator 24 or elongate component 38 may be coupled together with a keyed relationship. Additionally or alternatively, the blade or the blade shaft 62 may couple with the elongate component 38 and/or the second actuator 24 in any other suitable manner using, but not limited to, an over mold technique, a press or friction fit technique, an adhesive technique, a snap technique, a luer lock technique, a threaded coupling technique, and/or other suitable coupling techniques.

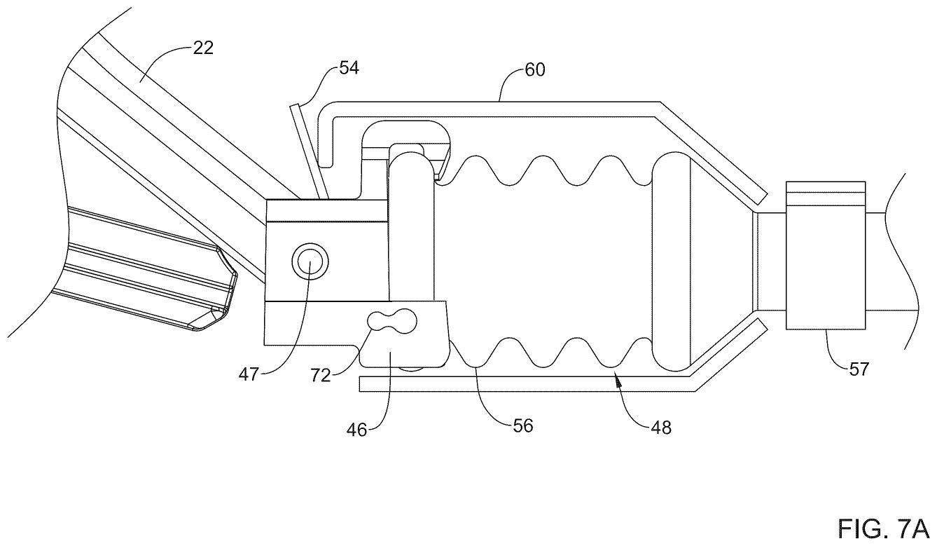

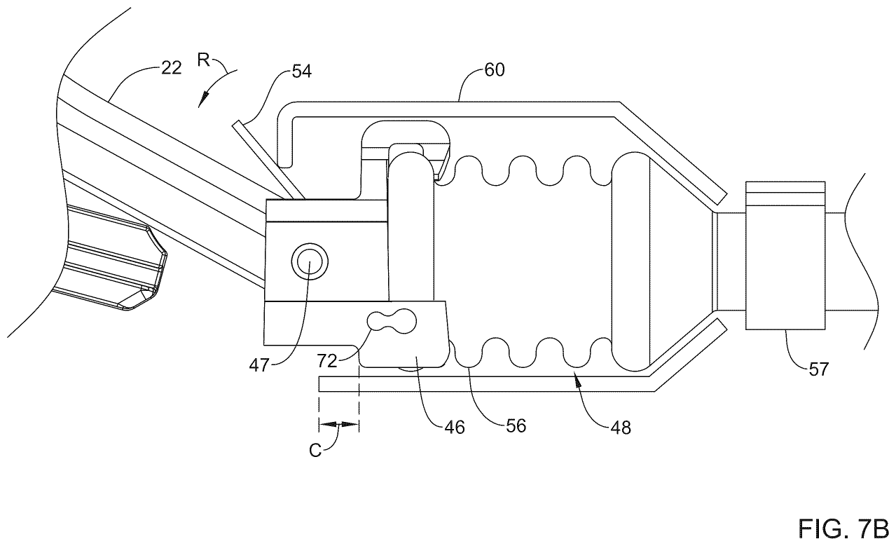

FIGS. 7A and 7B depict schematic side perspective views depicting adjustment or movement of the expansion device 48 in response to movement of the first actuator 22. In some cases, the connector 46 coupled with the expansion device 48 may optionally include an alignment component 72, where the alignment component may be configured to engage with alignment features of the housing 20 of the handle 12 to maintain linear movement of the expansion device 48 without rotational movement thereof. When included, the alignment component 72 may have any suitable shape and/or configuration including, but not limited to, a protrusion, an indent, a channel, a ledge, a structure with a circular cross-section, a structure with a peanut cross-section, and/or other suitable shape and/or configuration.

FIG. 7A depicts the expansion device 48 in a resting or relaxed, expanded position with the first actuator 22 in an unactuated position. When the expansion device 48 is in an expanded position and the first actuator 22 is in an unactuated position, the spring 54 may be in a relaxed position.

FIG. 7B depicts the expansion device 48, the connector 46, and the pivot pin 47 advanced or moved a distance C to a contracted position in response to actuation or movement of the first actuator 22 in the direction of arrow R to expand the expanders 16. When the expansion device 48 is in the contracted position and the first actuator 22 is in an actuated position, the spring 54 may bias the first actuator 22 to the unactuated position and may bias the expansion device 48 to the expanded position due to the spring 54 being tensioned in response to engagement with the carrier 60 and/or other components of the device 10 as the connector 46 and the expansion device 48 are adjusted to the contracted position. An over-center mechanism coupling between the first actuator 22 and the linkage(s) 40 (now shown in FIG. 7B) may prevent the bias of the spring 54 from returning the expansion device to the expanded position. Applying force to the first actuator 22 in the direction of the unactuated position may overcome the over-center mechanism and allow the spring 54 to bias the first actuator 22 to the unactuated position and the expansion device 48 to the expanded position.

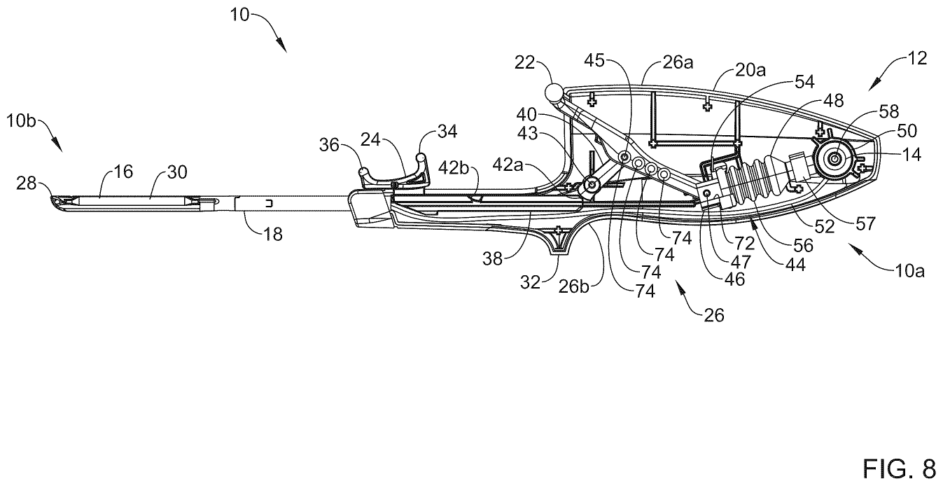

FIG. 8 depicts a schematic side view of an illustrative configuration of the device 10 with the second portion 20b of the housing 20 removed. Device 10 depicted in FIG. 8 may be similar to the device 10 depicted in FIG. 3 and common components are not re-described.

The first actuator 22 depicted in FIG. 8 may include one or more adjustment holes 74, such that a location at which the one or more linkages 40 engage the first actuator 22 may be adjusted. In some examples, the pivot pin 45 or a boss coupling the one or more linkages 40 with the first actuator 22 may be removed or separated from one or both of the linkage(s) 40 and the first actuator 22 and repositioned such that the linkage(s) 40 may be coupled with the first actuator 22 at a location of one other of the adjustment holes 74.

Allowing for adjustment of a location at which the linkage(s) 40 engages the first actuator 22 may facilitate altering an amount the expanders 16 are adjusted and/or altering a pressure at the expanders 16 in response to actuation of the first actuator 22. In some examples when expanders are balloons 30 or other suitable inflatable components, adjusting a location at which the linkage(s) engage the first actuator 22 may allow for an adjustment of an amount of fluid that is provided to the balloons 30 or other suitable inflatable components and thus, an adjustment of an amount of pressure provided at the balloons 30 or other suitable inflatable components. Further, utilizing a plurality of adjustment holes 74 may allow the same linkage(s) 40 and/or first actuators 22 to be used in devices that have different sizes of expanders 16 by changing at which adjustment hole 74 the linkage(s) 40 engages the first actuator 22 during assembly of the device 10. Alternatively or additionally, a user of the device 10 may be able to use the plurality of adjustment holes 74 to adjust a location at which the linkage(s) 40 engages the first actuator 22 and thus, adjust a volume of fluid provided to and/or a pressure at the expanders 16.

The first actuator 22 may have any suitable number of adjustment holes 74. For example, the first actuator 22 may include a one adjustment hole 74, two adjustment holes 74, three adjustment holes 74, four adjustment holes 74, five adjustment holes 74, and/or other any other suitable number of adjustment holes 74. In one example, the first actuator 22 may include four adjustment holes 74, but other suitable configurations are contemplated.

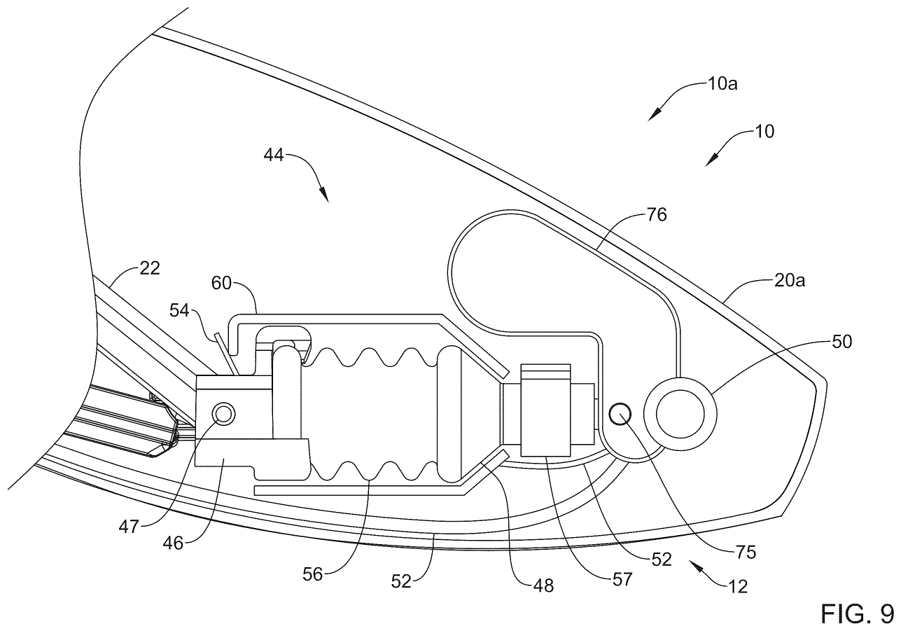

FIG. 9 depicts a schematic perspective of a portion of the proximal end 10a of an illustrative configuration of the device 10, where the second portion 20b of the housing 20 has been removed. FIG. 9 depicts the manifold 50 coupled with the expansion device 48 using the crimp 57, as discussed herein. The priming port 14 has been removed for clarity.

As discussed, the manifold 50 may take on a variety of shapes and/or configurations. In some examples, the manifold 50 may include a basin 76 (e.g., a pressure relief chamber and/or other suitable basin) configured to receive overflow fluid from one or more channels or fluid paths of the manifold 50. For example, as pressure rises in the expansion system 44, fluid may exit the channels of the manifold 50 and enter the basin 76 to avoid damaging the expansion system 44 (e.g., the expansion device 48, the tubes 52, the expanders 16, etc.) and/or causing fluid to leak out of the device 10 (e.g., via the priming port 14, etc.)

The basin 76 may include a cover, which has been omitted for clarity purposes. The cover of the basin 76 may be any suitable component configured to facilitate maintaining overflow fluid in the basin 76 and may include, but is not limited to, a component coupled with the basin 76, a component of the basin 76, a component monolithically formed with or as the basin 76, a portion of the housing 20 that mates with the basin 76, and/or other suitable component configured to cover or enclose the basin 76.

A pressure relief valve 75 in communication with the basin 76 may facilitate passing fluid from a channel or flow path of the manifold 50 and/or other suitable component of the expansion system 44 to the basin 76 when pressure in the flow path meets or goes beyond a threshold pressure. The pressure relief valve 75 may be any suitable type of valve that may control or limit the pressure in the expansion system 44 to avoid damaging the expansion system 44, avoid leaking fluid from the device 10 or components of the device 10, and to facilitate controlling pressures at and/or proximate to the expanders 16. Example suitable types of pressure relief valves 75 include, but are not limited to, spring-loaded pressure relief valves, wastegate regulator valves, modulating relief valves, snap relief valves, vacuum pressure relief valves, pilot operated relief valves, and/or other suitable pressure relief valves. In some examples, the pressure relief valve 75 may be or may include a spring-loaded pressure relief valve, but other suitable configurations are contemplated.

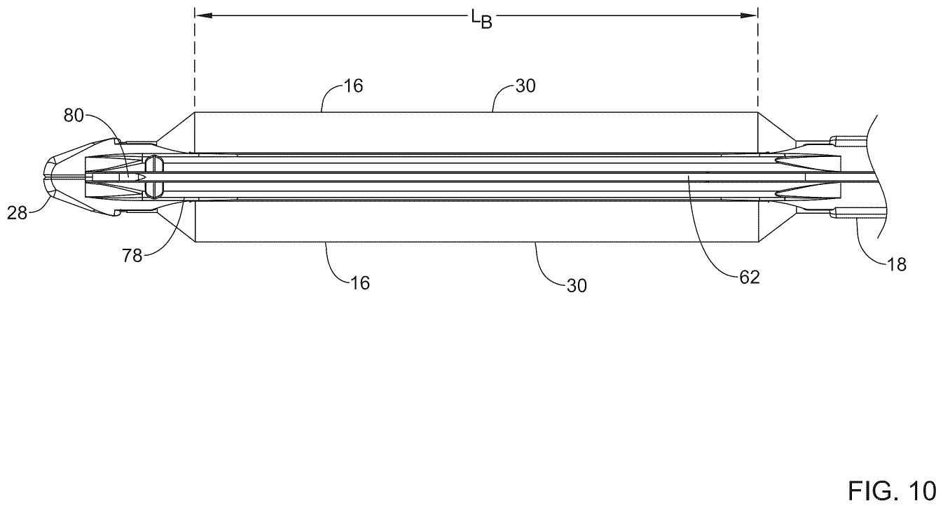

FIG. 10 schematically depicts a top view of a portion of the distal end 10b of the illustrative device 10 depicted in FIG. 1. The shaft 18 of the device 10 terminates at the distal tip 28 and defines an opening 78 through which the blade 80 may be exposed as the second actuator 24 (not shown in FIG. 10) coupled with the blade 80 (e.g., via the blade shaft 62) is adjusted or moved.

As discussed, the expanders 16 of the device 10 may extend along the shaft 18. In some configurations, the expanders 16 may extend along the opening 78 and/or a guideway defined, entirely or at least in part, by the shaft 18. In some examples, the device 10 may be configured such that the blade 80 of the device 10 is exposed along the opening 78 and/or the guideway and the expanders 16 may be configured to move tissue of a patient that is not to be cut by the blade 80 away from a cutting path of the blade 80 that is proximate the opening 78 and/or the guideway.

The expanders 16 may have any suitable length. In some examples, the expanders 16 may have a length LB configured to extend along at least an entirety of the cutting path of the blade 80. In one example, the blade 80 may have a cutting path that extends from an initial exposure location at or proximate a distal end of a body of the expanders 16 (e.g., at a proximal end of a distal cone of the expanders 16) and a fully covered or protected location at or proximate a proximal end of the body of the expanders 16 (e.g., at a distal end of a proximal cone of the expanders 16) and the length LB of the body of the expander 16 extends a same distance as the cutting path. In some examples, the length LB of the body of the expander 16 may be approximately 30 millimeters (mm) to approximately 40 mm when the device is configured to cut a carpal tunnel ligament, but other suitable lengths are contemplated for devices 10 configured to cut the carpal tunnel ligament and/or other soft tissue of patients.

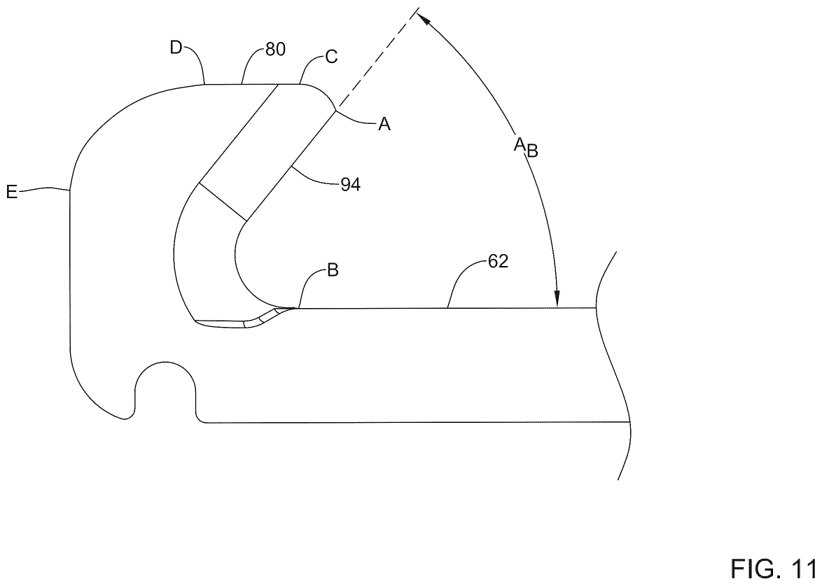

FIG. 11 schematically depicts a side view of the blade 80 and a portion of the blade shaft 62. The blade 80 may have a cutting surface 94 configured to engage and cut soft tissue of a patient. In some configurations, the cutting surface 94 may extend between point A to point B in FIG. 11, but other suitable cutting surface configurations are contemplated. In some examples, all other surfaces of the blade 80 not identified as the cutting surface 94 may be blunt surfaces that may be configured to prevent cutting of soft tissue.

The cutting surface 94 may be formed from one or more blade components. In some examples, the cutting surface 94 may be formed from a single blade component with two chamfered surfaces extending distally from the cutting surface 94. In some examples, two blade components may be used to form the cutting surface 94, where each blade component includes a single chamfered surface extending distally from a component cutting surface and the two blade components are coupled to one another to form the cutting surface 94 with the two component cutting surfaces such that two chamfered surfaces extend distally from the cutting surface 94.

At least a portion of the cutting surface 94 may form an angle AB relative to a longitudinal axis of the blade shaft 62. The angle AB may be any suitable angle. In some examples, the angle AB may be configured with a proximal lean or otherwise may be angled proximally, such that a portion of the cutting surface 94 proximate the top of the blade 80 is positioned further proximal than other portions of the cutting surface 94 closer to or proximate a bottom of the blade 80 or the blade shaft 62. In some examples, the angle AB may be between about thirty degrees and about sixty degrees. In one example, the angle AB may be about forty-five degrees, but other suitable configurations are contemplated.

As depicted in FIG. 11, a top of the blade 80 may have one or more rounded and/or blunt surfaces to prevent the blade 80 from inadvertently cutting tissue that is not intended to be cut (e.g., as the blade travels proximally and/or distally). Further, the rounded or blunt surfaces of the blade 80 may facilitate viewing the blade with an imaging system (e.g., with ultrasound imaging).

In some example configurations, the blade 80 may have a first blunt portion extending from point A to point C, where the first blunt portion may be rounded and configured to abut soft tissue without cutting the soft tissue. A second blunt portion of the blade 80 may extend from point C to point D and may generally have no or substantially no radius of curvature. A third blunt portion of the blade 80 may extend from point D to point E, where the third blunt portion may be rounded and configured to about soft tissue without cutting the soft tissue. Other suitable configurations of blunt portions of the blade 80 are contemplated.

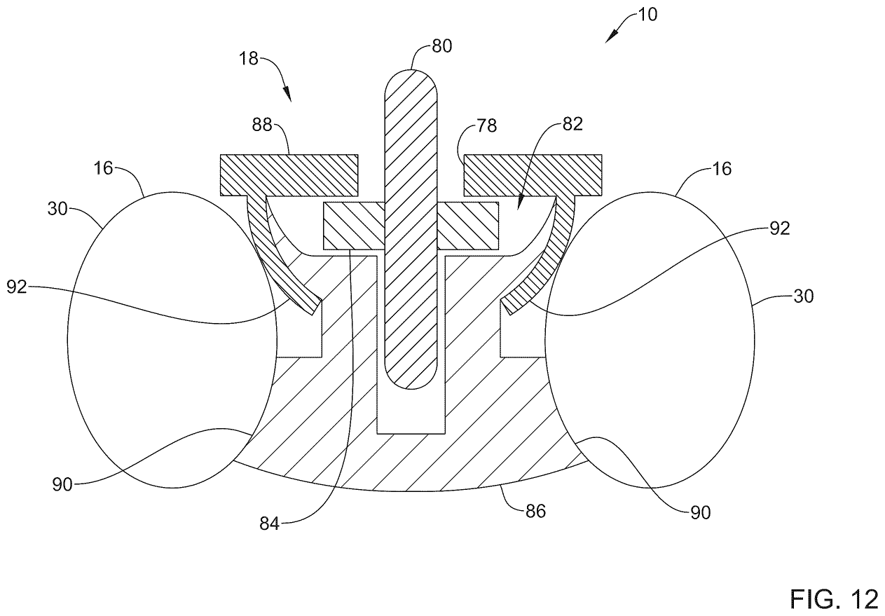

The shaft 18 of the device 10 may be made from any suitable components and may be manufactured in any suitable manner. In some examples, the shaft 18 may be configured to be rigid such that the shaft 18 withstands push forces as the shaft is inserted into a patient without buckling or bending (e.g., where the shaft 18 has a desired column strength), while being light weight so as to be easy to use, facilitate a balance of weight between the proximal end 10a and distal end 10b of the device 10, and mitigate injury to the patient as a procedure is performed. FIG. 12 schematically depicts a cross-section of an illustrative configuration the device 10, taken at a cross-section of the shaft 18, the expanders 16, and the blade 80, where the shaft 18 may be configured to have a desired rigidity and/or column strength, but also may be light weight.

As depicted in FIG. 12, the blade 80 is exposed through the opening 78 of the shaft 18 and the expanders 16, in the form of balloons 30, are in an expanded configuration. In addition to defining the opening 78, the shaft 18 may define a guideway 82 and a pin 84 coupled with and/or formed with the blade 80 may be positioned in and/or may travel within the guideway 82. Although the pin 84 is depicted in FIG. 12 as being a different material than the blade 80, the pin 84 may be monolithically formed with the blade 80 and/or may be made of the same material as the blade 80.

The shaft 18 may be formed with one or more materials. Example suitable materials for use in forming the shaft 18 include, but are not limited to, metals, polymers, and/or other suitable biocompatible materials. In some examples, the shaft 18 may be formed from metal materials and polymer materials, but other suitable configurations are contemplated.

The shaft 18 may be formed using one or more components. In some examples, the shaft 18 may be formed from a single high strength, light weight material. In some examples, the shaft 18 may be formed from a first light weight component and a second, high strength component. In one example, the shaft 18 may include a base component 86 formed from a first material, such as a polymer (e.g., a light weight polymer), and a cover component 88 formed from a second material, such as a metal (e.g., a rigid material or material having a desired column strength). Other suitable configurations are contemplated.

When the shaft 18 is formed from the base component 86 and the cover component 88, the components 86, 88 may be formed and/or combined in any suitable manner to form the opening 78 and the guideway 82. For example, the base component 86 and the cover component 88 may be formed using one or more suitable manufacturing techniques including, but not limited to, molding, over molding, machining, stamping, press-fitting, three-dimensional (3D) printing, liquid metal, crimping, reflowing, and/or other manufacturing techniques.

The base component 86 may be elongate and may extend along an entirety of or at least part of a length of the shaft 18. The base component 86 may define (e.g., at least partially define) an indented or recessed surface 90 extending along and accommodating or configured to mate with (e.g., have a radius of curvature that matches) an expanded expander 16, as depicted in FIG. 12. In some examples, the base component 86 may define at least a portion of the guideway 82 and/or a path along which the blade 80 may travel.

In some examples, including when the base component 86 is formed at least partially from a polymer, the base component 86 may be formed from a molding technique. Additional and/or alternative manufacturing techniques may be used

The cover component 88 may be elongate and may extend along an entirety of or at least part of a length of the shaft 18. The cover component 88 may define (e.g., at least partially define) the opening 78 for receiving an exposed blade 80 and the guideway 82 (e.g., with the base component 86) configured to receive the pin 84. In some examples, the cover component 88 may be configured to receiving an expanded expander 16.

As depicted in FIG. 12, the cover component 88 may couple with the base component 86 by wrapping one or more arms 92 around a portion of the base component 86 (e.g., around a portion of the base component 86 defining the guideway 82 and/or other portion of the base component 86). The cover component 88 may include one or more sets of laterally extending arms 92. In some examples, the cover component 88 may include two or more sets of laterally extending arms 92 at different axial locations to engage the base component 86 at discrete locations longitudinally spaced from one another. Alternatively, the arms 92 may extend along a length of cover component 88 and may be wrapped around an elongate portion of the base component 86. Alternatively or additionally, the cover component 88 may be coupled with the base component 86 in any other suitable manner.

In some examples including when the cover component 88 is formed at least partially from a metal, the cover component 88 may be formed from sheet metal using a stamping technique to cut a pattern of metal material for the cover component 88. Then, the pattern of metal material may be press-fit over the base component 86 to form a desired shape of the cover component 88 and couple the cover component 88 with the base component 86. Alternatively or additionally, the cover component 88 may be formed from a cut hypotube or other suitable metal tube and bent or otherwise fit around the base component 86 to form the shaft 18. In some examples, the base component 86 formed from a polymer material may be reflowed to facilitate coupling the cover component 88 with the base component 86. Additional and/or alternative manufacturing techniques may be used to form the cover component 88 and couple the cover component 88 with the base component 86.

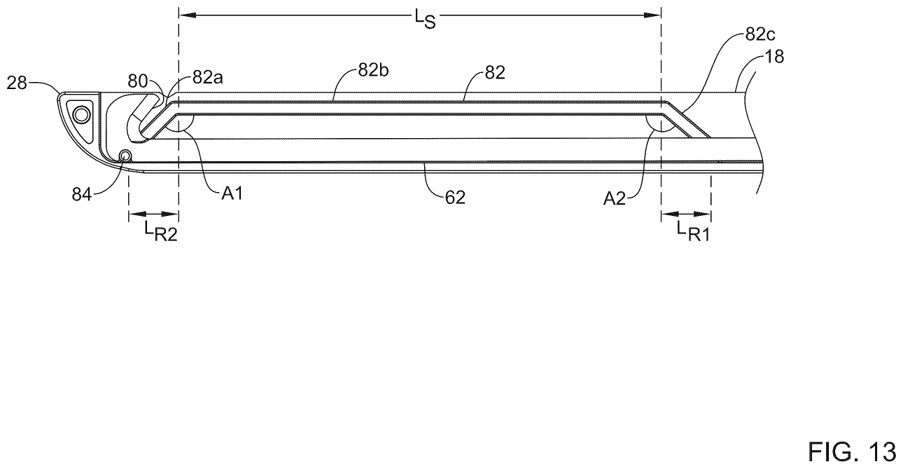

FIG. 13 depicts a schematic side of the distal end 10b of an illustrative configuration of the device 10, with a portion of the shaft 18 removed to show the blade 80, the blade shaft 62, the pin 84, and one side of the guideway 82. The pin(s) 84 may extend laterally from one or both sides of the blade 80 or the blade shaft 62 along the guideway 82 in the shaft 18 to facilitate adjusting the blade 80 between a protected position and an exposed or cutting position.

The guideway 82 may have any suitable configuration configured to facilitate moving or adjusting the blade 80 between a protected position and an exposed or cutting position. In some examples, the blade 80 may be configured to adjust or move along the guideway 82 between a distal, protected or unexposed first position, an exposed or cutting second position, and a proximal, protected or unexposed third position.

To facilitate adjusting the blade 80 between a protected or unexposed position and an exposed or cutting position, the guideway 82 may include one or more ramp portions (e.g., inclining/declining portions) and one or more steady portions (e.g., portions configured to maintain a height of the blade 80). In one example configuration of the guideway 82, the guideway 82 may have a first portion 82a that may be a ramp portion (e.g., tapering distally or inclining proximally), a second portion 82b extending proximally from the first portion 82a and configured to maintain a height of the blade 80, and a third portion 82c extending proximally from the second portion and that may be a ramp portion (e.g., tapering proximally or inclining distally). Other suitable configurations are contemplated.

The angles between the first and third portions 82a, 82c and the second portion 82b of the guideway 82 may be any suitable angles. In some configurations, the angles between the first and third portions 82a, 82c and the second portion 82b of the guideway 82 may be configured to facilitate cutting soft tissue of the patient and/or reducing forces on a user operating the device 10 to cut soft tissue of the patient. The angles A1 and A2 between the first and third portions 82a, 82c and the second portion 82b of the guideway 82 may be in a range of approximately thirty-five degrees to approximate sixty degrees and/or within one or more other suitable ranges. In one example, the angle A1 between the first portion 82a of the guideway 82 and the second portion 82b of the guideway 82 may be or may be about forty-five degrees and the angle A2 between the second portion 82b of the guideway 82 and the third portion 82c of the guideway 82 may be or may be about forty degrees. In another example, the angle A1 between the first portion 82a of the guideway 82 and the second portion 82b of the guideway 82 may be or may be about forty degrees and the angle A2 between the second portion 82b of the guideway 82 and the third portion 82c of the guideway 82 may be or may be about forty degrees. Other suitable configurations of the angles between the portions of the guideway 82 relative to one another are contemplated.

The portions of the guideway 82 may have any suitable length. In some examples, the lengths of the first portion 82a and the third portion 82c of the guideway 82 may be in a range of about 2.0 mm to about 5.0 mm and the length of the second portion 82b of the guideway 82 may be in a range of about 25 mm to about 45 mm. The guideway 82 may have a total length in a range of about 33 mm to about 55 mm. Other suitable distances for the lengths of the portions of the guideway 82 are contemplated.

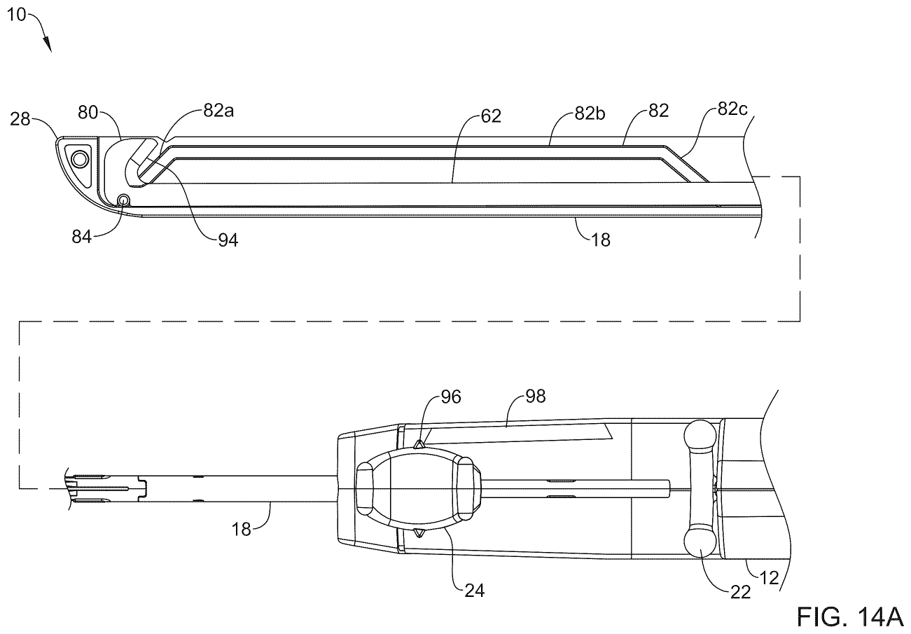

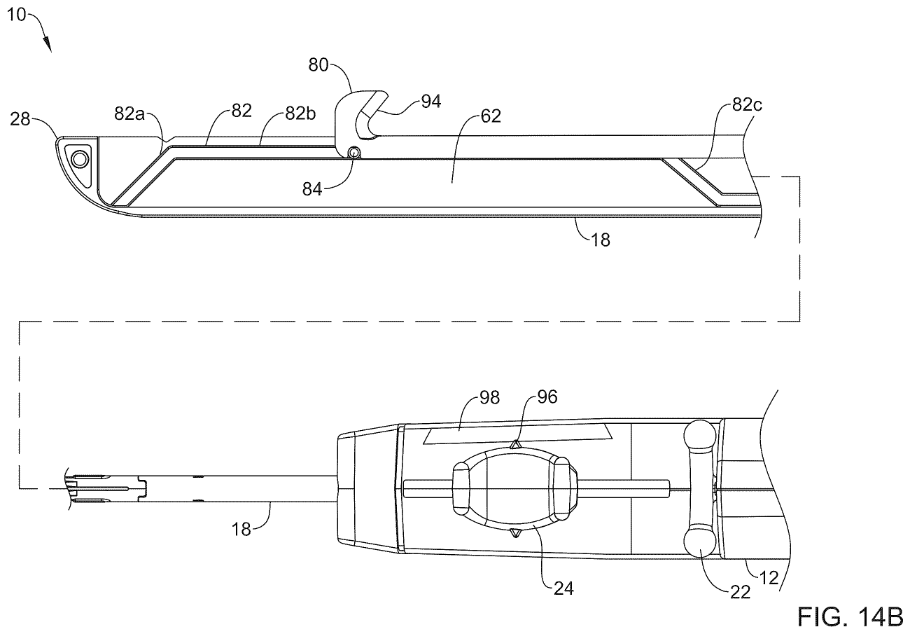

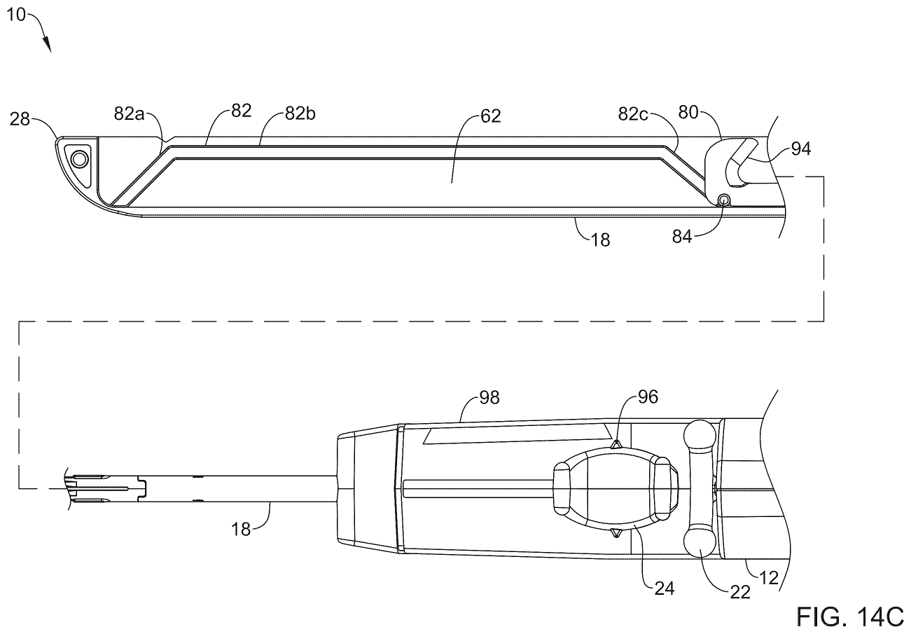

FIGS. 14A-14C schematically depict adjustment of the blade 80 between the protected positions and the exposed or cutting positions that may be used in a process of cutting soft tissue of a patient. As depicted in FIGS. 14A-14C, the second actuator 24 may include a pointer 96 that may point to a location along a position indicator 98 indicative of where the blade 80 is positioned. In some examples and as depicted in FIGS. 14A-14C, the position indicator 98 may be a replica of the guideway 82 to facilitate a user understanding where the blade 80 is located relative to the cutting path of the blade 80.

FIG. 14A depicts the second actuator 24 positioned fully distal with the pointer 96 located at a distal end of the position indicator 98, which may be indicative of the blade 80 being in a distal, protected or unexposed position at the first portion 82a of the guideway 82. FIG. 14B depicts the second actuator 24 positioned in an intermediate position with the pointer 96 located in a middle of the position indicator 98, which may be indicative of the blade 80 being in an exposed position in a middle of the second portion 82b of the guideway 82. As the blade travels along the second portion 82b of the guideway 82 in the exposed position, the cutting surface 94 of the blade 80 may engage soft tissue of the patient that is to be cut and cut the soft tissue as the blade traverses proximal along the second portion 82b of the guideway 82. FIG. 14C depicts the second actuator 24 positioned fully proximal with the pointer 96 located at a proximal end of the position indicator 98, which may be indicative of the blade 80 being in a distal, protected or unexposed position at the third portion 82c of the guideway 82.

The process of cutting soft tissue of a patient may be repeated by reversing the steps of FIGS. 14A-14C and then repeating the steps of FIGS. 14A-14C. The blunt top surfaces of the blade 80 may facilitate moving the blade 80 in a distal direction by deflecting tissue without cutting the tissue.

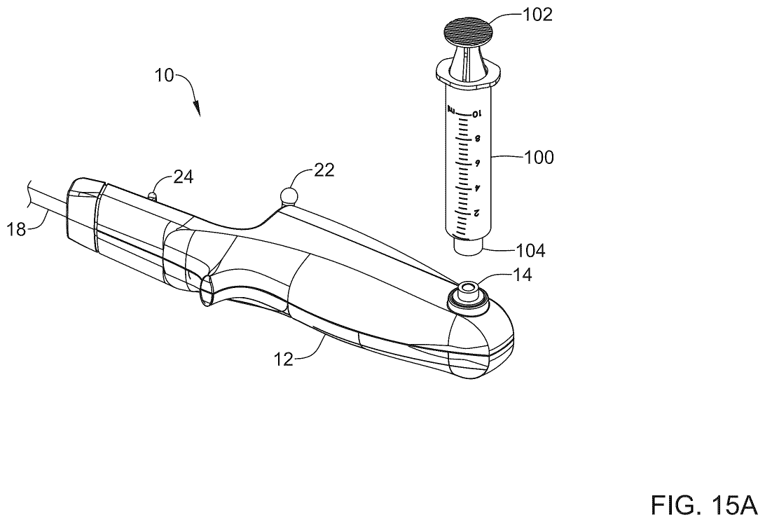

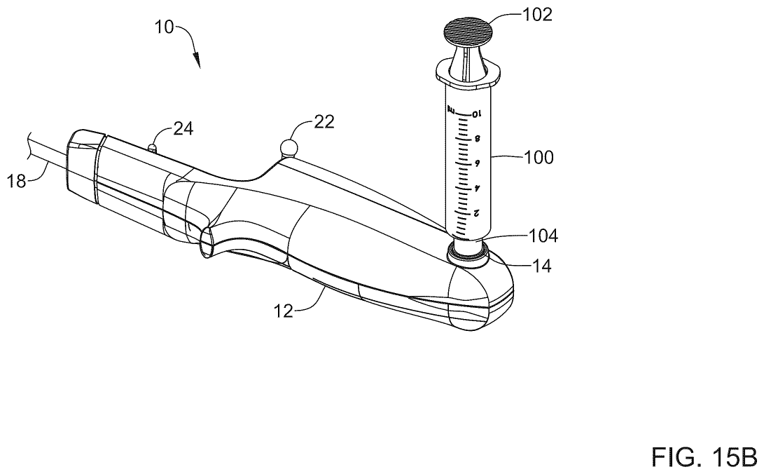

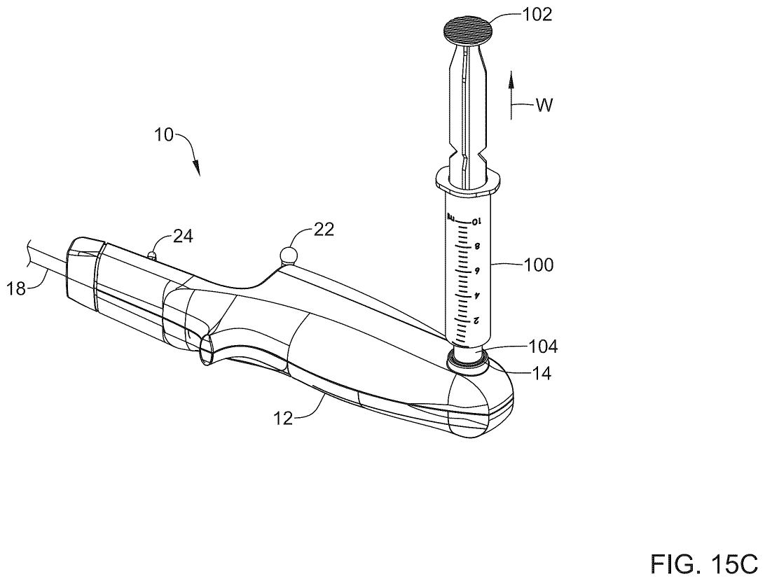

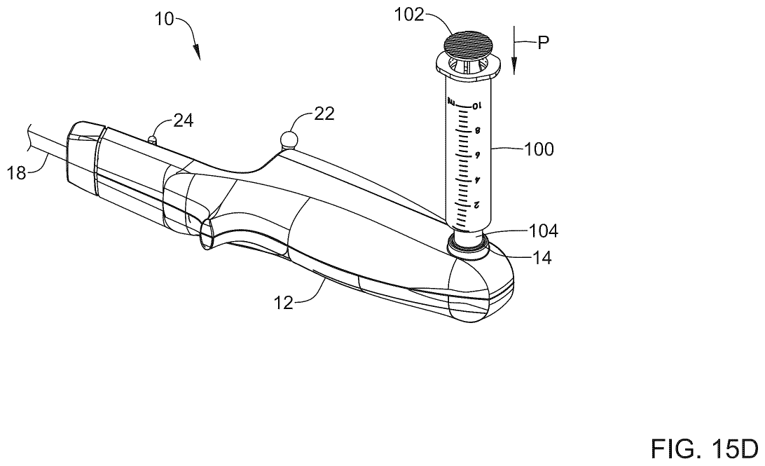

FIGS. 15A-15D schematically depict an illustrative technique for priming the expansion system 44 of the device 10 with fluid from a fluid transfer device. In some examples, a syringe 100 (e.g., a fluid source and/or a fluid transfer device) having a plunger 102 may engage the device 10 to transfer fluid from the syringe 100 to the device 10, where the transferred fluid may be used in controlling pressure at a distal end of the device 10 by expanding expanders 16 (e.g., by inflating balloons 30 and/or other suitable inflatable components). In some cases, the fluid transferred from the syringe 100 may be a liquid (e.g., saline, other suitable liquid, and/or other suitable non-compressible fluids). Suitable techniques for priming the expansion system 44 of the device 10 other than the techniques described with respect to FIGS. 15A-15D are contemplated

FIG. 15A schematically depicts the syringe 100 with a nozzle 104 aligned with the priming port 14. The syringe 100 may be at least partially filled with a fluid for use in the expansion system 44 of the device 10. In some configurations, the nozzle 104 and the priming port 14 may be configured to form a luer lock connection and/or other suitable type of connecting when engaged with one another.

FIG. 15B schematically depicts the nozzle 104 of the syringe 100 engaging or engaged with the priming port 14 of the device 10. In some examples, the device 10 may be supported by a flat surface (e.g., a plate, a table, etc.) as the nozzle 104 is engaged with the priming port 14 to facilitate the engagement. To reduce movement of the device 10 during engagement with the syringe 100, the handle 12 of the device 10 may have a flat surface that rests on the supporting flat surface such that the syringe 100 may be engaged with the priming port 14 with minimal movement of the device 10, but other suitable configurations are contemplated. As nozzle 104 engages the priming port 14, the nozzle 104 may interact with and open a valve in the priming port 14 to facilitate fluid flow between the expansion system 44 of the device 10 and the syringe 100. Although not depicted in FIG. 15B and not required, the device 10 may include a button or other suitable interface that may be actuated to initiate the connection between the syringe 100 and the device 10.

Once the syringe 100 has been engaged or coupled with the device 10 at the priming port 14 and before inserting fluid into the expansion system 44 of the device 10, a vacuum pressure may be created in the expansion system 44 and the syringe 100. For example, the vacuum pressure may be created by withdrawing the plunger 102 in the direction of arrow W as a user applies a withdrawing force to the plunger 102. Withdrawing the plunger 102 before providing the fluid in the syringe 100 to the expansion system 44 of the device 10, may draw fluid (e.g., air and/or other suitable fluid) into the syringe 100 from the expansion system of the device 10 and create a vacuum in the expansion system 44 of the device 10.

Once a user has released the plunger 102 and removed the withdrawing force from the plunger 102, the vacuum in the expansion system 44 of the device 10 may pull the plunger 102 in the direction P to an inserted position, as shown in FIG. 15D, and cause the liquid in the syringe 100 to enter the expansion system 44 prior to any air or other fluid in the syringe 100 enters the expansion system 44. The liquid in the syringe 100 may be a sufficient amount to fill the expansion system 44 of the device 10. However, if it is believed air or other compressible fluid remains in the expansion system 44, the steps discussed with respect to FIGS. 15B-15D may be repeated until a user is satisfied that no air or compressible fluid or at least only an acceptable amount of air or compressible fluid remains in the expansion system 44 of the device 10. As discussed, it may be beneficial to remove as much air or other compressible fluid from the system as possible, as air in the expansion system 44 that travels to the expanders 16 may impair the ability to create a predictable pressure at the expanders 16 and/or to image the expanders 16 when the distal end 10b of the device 10 is positioned in a patient due to air being generally transparent on ultrasound images and/or other images that may be used in soft tissue cutting procedures.

FIGS. 16A-16D depict an illustrative technique or method of operating a device 10 for cutting body tissue (e.g., soft tissue) of a patient, where a second portion 20b of the housing 20 has been removed to schematically depict movements within the handle 12 of the device 10 as the first actuator 22 and the second actuator 24 are adjusted. When the device 10 has been primed and is in the configuration depicted in FIG. 16A with the first actuator 22 in an unactuated position and the second actuator 24 distally positioned such that the blade 80 is in a first protected position, the distal end 10b of the device 10 may be inserted into a target area of a patient that is proximate a body tissue to be cut. In some examples, the shaft 18 may be inserted into the hand and through a carpal tunnel of the hand when the device is to be used in a carpal tunnel release procedure on the hand of the patient. In some examples, the shaft 18 may be inserted into the patient such that the expanders 16 have been fully inserted into the patient, but this is not required.

Once the distal end 10b of the device 10 has been inserted into the patient, the first actuator 22 may be actuated or adjusted to a fully actuated position. Actuating the first actuator 22 may include moving the first actuator 22 in the direction of arrow R to the fully actuated position, which may position a joint between the one or more linkages 40 and the first actuator 22 in an over-center position such that the first actuator 22 remains in the actuated position despite being biased to the unactuated position depicted in FIG. 16A. Actuating the first actuator 22 and positioning the linkage(s) 40 and the first actuator 22 in the over-center position, may disengage linkage(s) 40 from the first engagement portion 42a of the elongate component 38 and unlock the second actuator 24 coupled with the blade 80 to allow the second actuator 24 and the blade 80 to adjust longitudinally.

Actuating or adjusting the first actuator 22 may result in compacting or contracting the expansion device 48 in the direction of arrow E, which may cause fluid to travel through the tubes 52 to the inflatable expanders 16 (e.g., the balloons 30 or other inflatable components) and inflate or expand (e.g., laterally and/or radially enlarge) the expanders 16, as depicted in FIG. 16B. Expanding or inflating the expanders 16 extending along the shaft 18 and the guideway 82 or cutting path of the blade 80 may result in moving tissue (e.g., soft tissue or other suitable tissue) of the patient that is not intended to be cut laterally away from the cutting path of the blade 80.

Once the expanders 16 have been inflated and expanded and the linkage(s) 40 disengage the elongate component 38 to unlock the second actuator 24, the second actuator 24 may be adjusted proximally to adjust the blade 80 from a protected first position as in FIGS. 16A and 16B to an exposed or cutting second position, as depicted in FIG. 16C. The second actuator 24 may be further adjusted to a fully proximal position to move the blade 80 from the exposed or cutting second position to a protected third position, as depicted in FIG. 16D. As the blade 80 traverses in the exposed or cutting second position along the shaft 18 between the protected first position and the protected third position, the blade 80 (e.g., the cutting surface of the blade 80) may cut a transverse carpal tunnel ligament of the patient.

Once the blade 80 is in the protected third position, a decision as to whether the body tissue has been sufficiently cut may be made. When the body tissue has not been sufficiently cut, the steps of FIGS. 16B and 16C may be reversed and then repeated in the order discussed until the body tissue has been sufficiently cut. When the body tissue has been sufficiently cut and the blade is in the protected third position with the second actuator 24 in the fully proximal position, the first actuator 22 may be adjusted to an unactuated position, as shown in FIG. 16E. Positioning the first actuator 22 in the unactuated position may release a joint between the one or more linkages 40 and the first actuator 22 from the over-center position such that the first actuator 22, which may allow the linkage(s) 40 to engage the second engagement portion 42b of the elongate component 38 and lock the second actuator 24 in the proximal position and the blade in the protected third position. Further, adjusting the first actuator 22 to the unactuated position may result in expanding the expansion device 48, which may cause fluid to travel from the expanders 16 through the tubes 52 to the manifold 50 and/or the expansion device 48 and deflate the expanders 16, as depicted in FIG. 16E. Once the second actuator 24 has been locked in the proximal position with the blade 80 locked in the protected third position and the expanders 16 have been deflated or contracted, the device 10 may be removed from the patient and the insertion point on the patient may be sutured or otherwise closed.

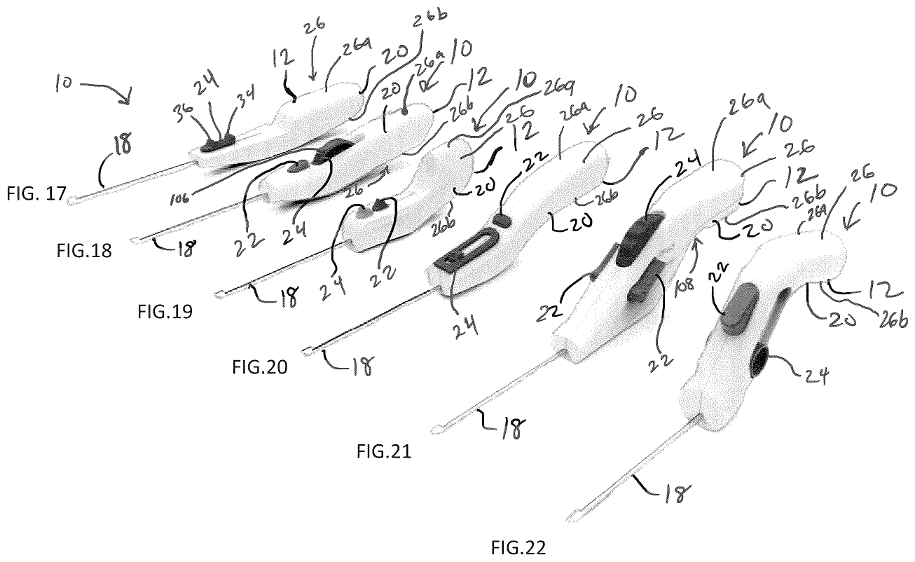

FIGS. 17-22 are perspective views of six illustrative configurations of the device 10, each with a different configuration of the handle 12 and a different configuration of the actuators 22, 24. The configurations of the devices 10 depicted in FIGS. 17-22 may include the shaft 18 extending distally from the handle 12 and the housing 20 defining an outer surface of the handle 12, including the grip portion 26. Although the expanders 16, the blade 80, and the blade shaft 62 are not depicted in FIGS. 17-22, the devices 10 may include these components and/or other components of the configurations of the device 10 discussed herein. Any other suitable mechanism(s) may be used in addition to and/or alternative to the mechanisms of configurations of the device 10 depicted in FIGS. 17-22.

The handles 12 of the configurations of the devices 10 depicted in FIGS. 17-22 may be held by user in any suitable manner. In some examples, the handles 12 may be configured to be used with a single hand. For example, in general, a user may grasp the handle 12 with the palm of its hand on the top surface 26a and one or more fingers of the hand on the bottom surface 26b of the grip portion 26 and adjust the first actuator 22 and/or the second actuator 24 with a thumb of the hand engaging handle 12. Other configurations of a user engaging the handle 12 with a hand are contemplated.

FIG. 17 schematically depicts an illustrative configuration of the device 10 with an elongate grip portion 26. The top surface 26a of the grip portion 26 may be relatively straight or flat, while the bottom surface 26b of the grip portion 26b may angle toward the top surface in a distal to proximal direction such that a height of the handle 12 may be reduced in the distal to proximal direction of the grip portion 26.

The configuration of the device 10 in FIG. 17 may omit the first actuator 22 and the expanders 16 may be expanded or inflated in response to movement of the second actuator 24. As depicted in FIG. 17, the second actuator 24 may have a slider configuration that may be moved longitudinally (e.g., moved proximally and/or distally) along the handle 12 to adjust the blade 80 between one or more protected positions in which a cutting surface is not exposed to tissue and one or more cutting positions in which the cutting surface is exposed to a target tissue of a patient. In some configurations, the second actuator 24 in the slider configuration may have the proximal extension 34 and the distal extension 36 spaced distally from the proximal extension 34 with a gap between the proximal extension 34 and the distal extension 36, such that a user adjust the second actuator 24 proximally and/or distally with a thumb of the hand engaging the proximal extension 34, the distal extension 36, a space between the proximal extension 34 and the distal extension 36, and/or other portion of the second actuator 24.

FIG. 18 schematically depicts an illustrative configuration of the device 10 with an elongate grip portion 26. The top surface 26a and the bottom surface 26b of the grip portion 26 may both be relatively straight or flat such that a height of the handle 12 may be substantially constant along a length of the grip portion 26. In some examples, the top surface 26a of the grip portion 26 may extend proximally a distance further than a distance the bottom surface 26b extends proximally, but other suitable configurations are contemplated.

The configuration of the device 10 in FIG. 18 may include a button configuration of the first actuator 22, where the first actuator 22 may be positioned distal of the second actuator 24 and may be pushed or pressed to expand or inflate the expanders 16. As depicted in FIG. 18, the second actuator 24 may have a wheel configuration and may be rotated to adjust the blade 80 between one or more protected positions in which a cutting surface is not exposed to tissue and one or more cutting positions in which the cutting surface is exposed to a target tissue of a patient. In some configurations, the second actuator 24 in the wheel configuration may have a tab 106. A user may actuate the first actuator 22 with a thumb of the hand engaging handle 12 and adjust the second actuator 24 proximally and/or distally with the thumb engaging the tab or other portion of the second actuator 24. In some examples, a rotational location of the tab 106 may be indicative of a position of the blade 80.

FIG. 19 schematically depicts an illustrative configuration of the device 10 with a short or abbreviated grip portion 26. The top surface 26a of the grip portion 26 may be relatively straight or flat, while the bottom surface 26b of the grip port 26b may angle toward the top surface in a distal to proximal direction such that a height of the handle 12 may be reduced in the distal to proximal direction of the grip portion 26. Relative to the lengths of the grip portions 26 of the configurations of the devices 10 in FIGS. 17 and 18, the length of the grip portion 26 in the configuration of the device depicted in FIG. 19 may be reduced.

The configuration of the device 10 in FIG. 19 may have a dual slider configuration, where the first actuator 22 may have be a proximal slider that may be moved proximally to expand or inflate the expanders 16. As depicted in FIG. 18, the second actuator 24 may be a distal slider that may be moved longitudinally (e.g., proximally and/or distally) after or as the first actuator 22 is adjusted proximally to adjust the blade 80 between one or more protected positions in which a cutting surface is not exposed to tissue and one or more cutting positions in which the cutting surface is exposed to a target tissue of a patient. A user may grasp the bottom surface 26b of the grip portion 26 with less than all of the fingers of the user due to the short length of the grip portion 26 and may adjust the first actuator 22 and the second actuator 24 proximally and/or distally with a thumb of the hand engaging handle 12.

FIG. 20 schematically depicts an illustrative configuration of the device 10 with an elongate grip portion 26. The top surface 26a and the bottom surface 26b of the grip portion 26 may both be relatively straight or flat such that a height of the handle 12 may be substantially constant along a length of the grip portion 26. However, one or both of the top surface 26a and the bottom surface 26b may extend away from the other as the surfaces 26a, 26b extend proximally such that the height of the handle 12 increases in a proximal direction. In some examples, the grip portion 26 may be raised up relative to a location of the handle 12 at or proximate the first actuator 22 and/or the second actuator 24.

The configuration of the device 10 in FIG. 20 may include a button configuration of the first actuator 22, where the first actuator 22 may be positioned proximal of the second actuator 24 and may be pushed or press to expand or inflate the expanders 16. As depicted in FIG. 20, the second actuator 24 may have a slider configuration and may be moved laterally (e.g., moved proximally and/or distally) to adjust the blade 80 between one or more protected positions in which a cutting surface is not exposed to tissue and one or more cutting positions in which the cutting surface is exposed to a target tissue of a patient. In some configurations, a ring may be located around a path of travel of the second actuator 24. A user may actuate the first actuator 22 with a thumb of the hand grasping the handle 12 and adjust the second actuator 24 proximally and/or distally with the thumb.

FIG. 21 schematically depicts an illustrative configuration of the device 10 with an elongate grip portion 26. The top surface 26a and the bottom surface 26b of the grip portion 26 may both be relatively straight or flat such that a height of the handle 12 may be substantially constant along a length of the grip portion 26. However, the bottom surface 26b may define a reduced depth portion 108 configured to receive a finger of a hand of a user while one or more other fingers of the hand of the user engage a portion of the bottom surface 26b proximal of the reduced depth portion 108. In some examples, the grip portion 26 may be raised up relative to a location of the handle 12 at or proximate the first actuator 22 and/or the second actuator 24.

The configuration of the device 10 in FIG. 21 may include a dual lever configuration of the first actuator 22, where the first actuator 22 may be formed from two levers extending laterally from the housing 20 and extending at least partially distal of and lateral to the second actuator 24, where the first actuator 22 may be pushed or press to expand or inflate the expanders 16. As depicted in FIG. 18, the second actuator 24 may have a wheel configuration and may be rotated to adjust the blade 80 between one or more protected positions in which a cutting surface is not exposed to tissue and one or more cutting positions in which the cutting surface is exposed to a target tissue of a patient. In some configurations, the second actuator 24 in the wheel configuration may include indents and/or protrusions to facilitate a user engaging the wheel. A user may grasp the handle 12 with one or more fingers in the reduced depth portion 108 of the grip portion 26, when included, and may actuate the first actuator 22 with a thumb of the hand grasping the handle and adjust the second actuator 24 proximally and/or distally with the thumb. In some examples, the lateral position of the dual levers may facilitate a use of the device 10 with a left hand or a right hand of the user.

FIG. 22 schematically depicts an illustrative configuration of the device 10 with an elongate grip portion 26. The top surface 26a and the bottom surface 26b of the grip portion 26 may both be relatively straight or flat such that a height of the handle 12 may be substantially constant along a length of the grip portion 26. In some examples, the grip portion 26 may be raised up and angled downward relative to a location of the handle 12 at or proximate the first actuator 22 and/or the second actuator 24 that may be angled upward.

The configuration of the device 10 in FIG. 21 may include a button configuration of the first actuator 22, where the first actuator 22 may be pressed or pushed to expand or inflate the expanders 16. As depicted in FIG. 21, the second actuator 24 may have a dual slider configuration, where the second actuator 24 may be formed from two sliders at lateral sides of the housing 20 that are configured to slide between a location distal of the first actuator 22 and a location proximal of the first actuator 22 such that the second actuator 24 may be moved proximally and/or distally to adjust the blade 80 between one or more protected positions in which a cutting surface is not exposed to tissue and one or more cutting positions in which the cutting surface is exposed to a target tissue of a patient. In some configurations, the sliders of second actuator 24 may have indents or may be hollow to facilitate a user engaging the sliders. A user may actuate the first actuator 22 with a thumb of the hand grasping the handle 12 and adjust the second actuator 24 proximally and/or distally with the thumb. In some examples, the lateral position of the dual sliders may facilitate a use of the device 10 with a left hand or a right hand of the user.

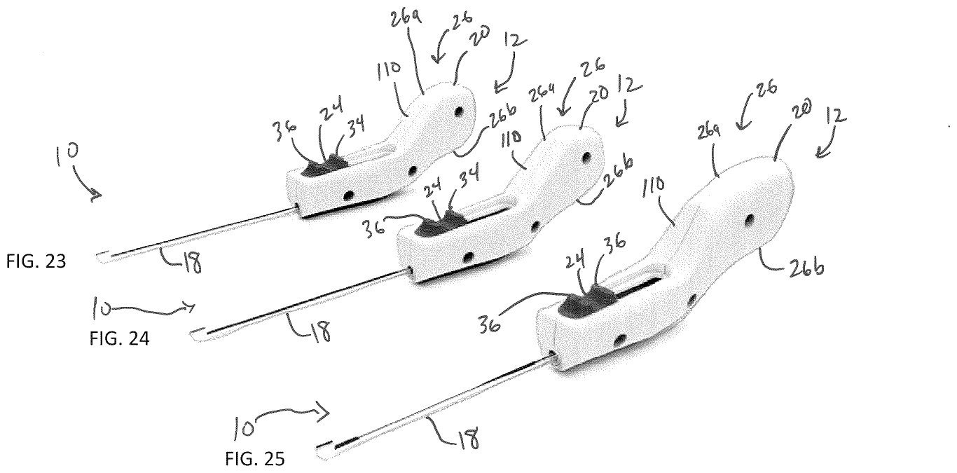

FIGS. 23-25 are schematic perspective views of three different configurations of the device. Each of the configurations of the device 10 depicted in FIGS. 23-25 may include a second actuator 24 similar to the configuration of the second actuator 24 depicted in and discussed with respect to FIG. 17, but with different configurations of the handles 12 at or proximate the grip portion 26. Other suitable configurations of the handle 12 are contemplated.

FIG. 23 schematically depicts an illustrative configuration of the device 10 with the handle 12 having a short grip portion 26 relative to the grip portion 26 in FIG. 17 and FIG. 25. Further, the handle 12 may include an inclined portion 110 extending proximally from a portion of the handle 12 at which the second actuator 24 is configured to adjust, such that at least a portion of the grip portion 26 may be raised relative to the portion of the handle 12 at which the second actuator 24 may be configured to adjust. In some configurations, the inclined portion 110 may be part of the grip portion 26 and a user may contact and/or grasp at least part of the inclined portion 110 when using the device 10 in a procedure.

FIG. 24 schematically depicts an illustrative configuration of the device 10 with the handle 12 having a short grip portion 26, similar to a length of the grip portion 26 depicted in FIG. 23. Further, the handle 12 may include the inclined portion 110 extending proximally from a portion of the handle 12 at which the second actuator 24 is configured to adjust, such that at least a portion of the grip portion 26 may be raised relative to the portion of the handle 12 at which the second actuator 24 may be configured to adjust. The inclined portion 110 of the handle 12 depicted in FIG. 24 may be longer than the inclined portion 11 of the handle 12 depicted in FIG. 23. As such, the portion of the grip portion 26 proximal of the inclined portion 110 depicted in FIG. 24 may be shorter than the portion of the grip portion 26 extending proximal of the inclined portion 110 depicted in FIG. 23 in view of the grip portions 26 in FIGS. 23 and 24 having the same or similar lengths.

FIG. 25 schematically depicts an illustrative configuration of the device 10 with the handle 12 having an elongate grip portion 26 relative to the length of grip portions 26 depicted in FIGS. 23 and 24. Further, the handle 12 may include the inclined portion 110 extending proximally from a portion of the handle 12 at which the second actuator 24 is configured to adjust, such that at least a portion of the grip portion 26 may be raised relative to the portion of the handle 12 at which the second actuator 24 may be configured to adjust. The inclined portion 110 of the handle 12 depicted in FIG. 25 may be a similar length as a length of the inclined portion 110 of the handle 12 depicted in FIG. 23. The portion of the grip portion 26 proximal of the inclined portion 110 depicted in FIG. 24 may be longer than the portion of the grip portion 26 extending proximal of the inclined portion 110 depicted in FIGS. 23 and 24.

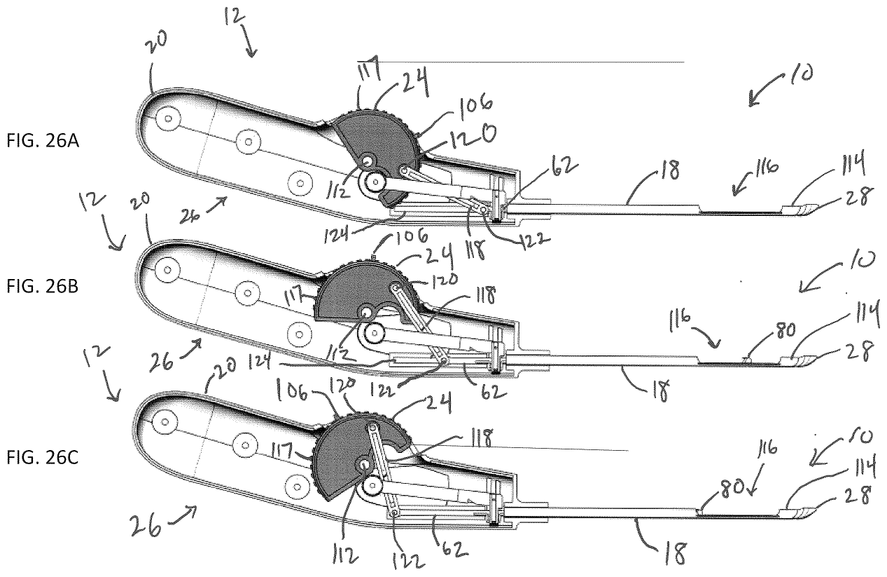

FIGS. 26A-26C are schematic side views of an illustrative configuration of the device 10 with one side of the housing 20 removed to show an internal blade retracting mechanism of the device 10. As depicted in FIGS. 26A-26C, the second actuator 24 may have a wheel configuration, where the second actuator 24 may rotate about an axis of rotation at a pivot component (e.g., a pin, a protrusion, an indent, etc.) 112 to advance and retract the blade 80 of the device 10 to and from a garage 114 at or proximate the tip 28 of the shaft 18. The blade 80 may be in a protected position when in the garage 114 and may be in an exposed or cutting position when at a window 116 of the shaft 18.

In some examples, the wheel configuration of the second actuator 24 may include teeth 117 that are part of a ratchet system, but other configurations are contemplated. In some examples, the teeth may be configured to engage the housing 20 of the handle 12 so as to give tactile and/or audible feedback to a user as the second actuator is moved or adjusted. Further, the second actuator 24 may include a tab 106 that may be engaged by a user and/or may be used as an indication of a position of the blade 80 as the second actuator 24 is adjusted (e.g., rotated about the pin 112 and/or adjusted in one or more other suitable manners).

As depicted in FIGS. 26A-26C, the second actuator 24 may be coupled with the blade shaft 62 of or coupled with the blade 80 via one or more blade linkages 118. A first end of the blade linkage 118 may couple with the second actuator 24 at a first pivot location 120 between the pivot component 112 and an outer circumference of the second actuator 24 using any suitable pivot connection. A second end of the blade linkage 118 may be coupled with the blade shaft 62 at a second pivot location 122 using any suitable pivot connection. In some configurations, the blade shaft 62, a pivot component (e.g., pin, protrusion, indent, etc.) used to couple the blade shaft 62 and the blade linkage 118, and/or other suitable components may be configured to travel linearly in a channel 124 as the second actuator 24 is rotated to maintain linear movement of the blade shaft 62 and/or the blade 80.

FIG. 26A schematically depicts the second actuator 24 rotated forward such that the blade 80 (not shown) may be housed in the garage 114 in a protected first position. FIG. 26B schematically depicts the second actuator 24 rotated in a proximal direction a distance such that the blade 80 may be positioned at the window 116 proximal of the garage 114 and at a cutting or exposed second position. FIG. 26C schematically depicts the second actuator 24 rotated in a fully proximal direction such that the blade 80 may be in a protected third position at a proximal end of the window 116 and at least partially covered by the shaft 18 (e.g., such that a cutting surface of the blade 80 is covered and/or protected). Other suitable configurations for adjusting or moving the blade 80 are contemplated.

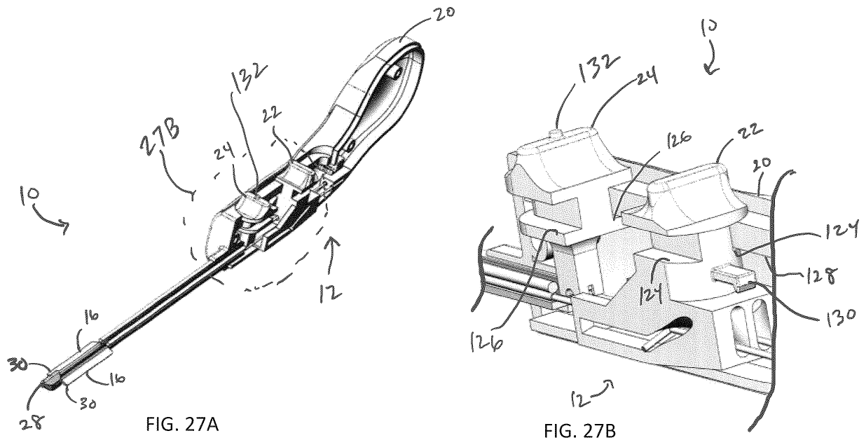

FIGS. 27A and 27B are schematic perspective views of an illustrative configuration of the device 10, where the first actuator 22 and the second actuator 24 may be configured as a dual slider mechanism for expanding the expanders 16 (e.g., inflating the balloons 30, etc.) and adjusting a position of the blade 80 (not shown in FIGS. 27A and 27B). In some examples, the first actuator 22 and the second actuator 24 of the configuration of the device 10 depicted in FIGS. 27A and 27B may be the same as or similar to the first actuator 22 and the second actuator 24 depicted in FIG. 19, but other suitable configurations are contemplated.

FIG. 27A schematically depicts a front perspective view of the device 10 with portions of the device 10 removed to show the first actuator 22 and the second actuator 24. The first actuator 22 may be located proximal of the second actuator 24 such that the second actuator 24 may be adjusted proximally to move the blade 80 from a protected first position to a cutting or exposed second position only after or while the first actuator 22 is adjusted in the proximal direction. Other suitable configurations are contemplated.

FIG. 27B schematically depicts a magnified back perspective view of the device 10, taken from the circle 27B in FIG. 27A. As depicted in FIG. 27B, the first actuator 22 may include one or more protrusions or ledges 124 and the second actuator 24 may include one or more protrusions or ledges 126 that are configured to engage a channel 128 of the housing 20 to facilitate maintaining linear movement of the first actuator 22 and the second actuator 24 as the first and second actuators 22, 24 are adjusted or move relative to the housing 20. Alternatively or additionally, the housing 20 may include the ledges or other suitable protrusions and the first actuator 22 and/or the second actuator 24 may include a channel.

The first actuator 22 may include one or more latches 130 configured to engage the housing 20 or other suitable portion of or component at the handle 12. In some examples, the latch 130 may be located at a proximal end of the first actuator 22 such that when the first actuator 22 is withdrawn proximally a full travel distance ensuring expansion of the expanders 16, the latch 130 may releasably couple to or relative to the housing 20 to prevent the expanders 16 from contracting until a desired time (e.g., until the second actuator 24 has been moved so as to position the blade 80 in a protected position).

In some examples, the first actuator 22 or the second actuator 24 may include a nub 132 (e.g., the second actuator 24, as depicted in FIGS. 27A and 27B) or other suitable tactile component. When included, the nub 132 may be utilized by a user of the device 10 to identify which slider is the second actuator 24 (e.g., in FIG. 27B, the slider with the nub 132) and which slider is first actuator 22 (e.g., in FIG. 27B, the slider without the nub 132). Other suitable techniques for identifying the first actuator 22 and the second actuator 24 may be utilized or omitted, as desired.

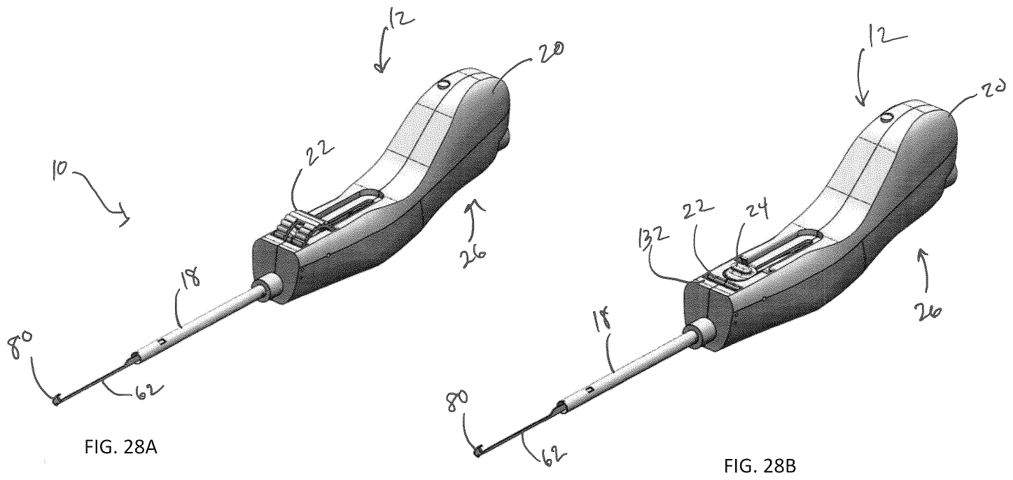

FIGS. 28A and 28B depict schematic perspective views of an illustrative configuration of the device 10, with a rotationally actuated first actuator 22 and a slider second actuator 24. A portion of the shaft 18 has been removed to depict the blade shaft 62 and the blade 80.

FIG. 28A depicts the first actuator 22 in an unactuated position. When in the unactuated position, the first actuator 22 may block a user from accessing the second actuator 24, which may be located under or below the first actuator 22. In some examples, the first actuator 22 may have ridges to facilitate a user engaging an outer surface of the first actuator 22 and/or for other purpose.

FIG. 28B depicts the first actuator 22 rotated distally in a fully actuated position to expand the expanders 16 (not shown in FIGS. 28A and 28B). The first actuator 22 may be rotated into the slot 132 extending through the housing 20 and located distal of the second actuator 24, but other suitable configurations are contemplated. When the first actuator 22 has been positioned in the fully actuated position, the second actuator 24 may be exposed and a user may engage the second actuator 24 for longitudinal adjustment along the handle 12 to longitudinally move or position the blade 80, as desired.

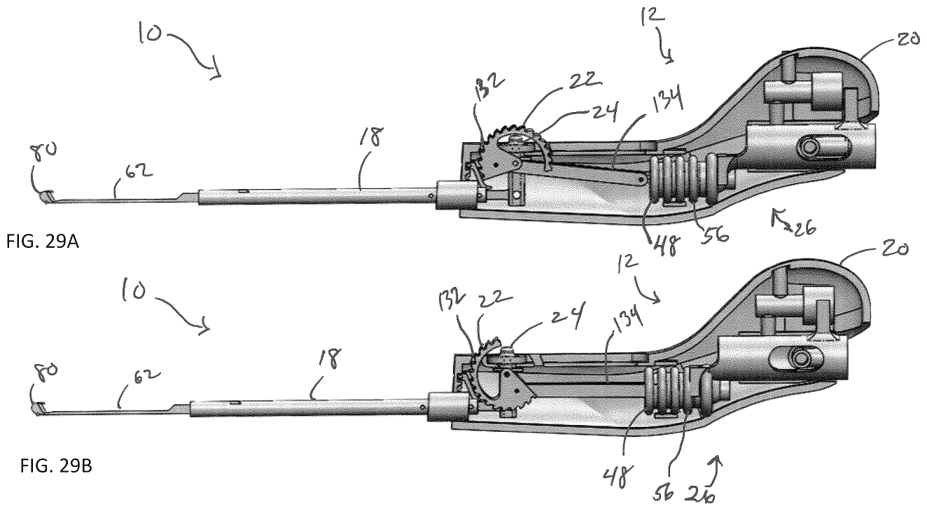

FIGS. 29A and 29B depict schematic side views of the device 10 depicted in FIGS. 28A and 28B, with components of the device 10 removed, including a portion of the housing 20. FIG. 29A corresponds to the orientation of the device 10 in FIG. 28A and FIG. 29B corresponds to the orientation of the device 10 in FIG. 28B.

FIG. 29A schematically depicts the first actuator 22 in the unactuated position, as discussed with respect to FIG. 28A. When in the unactuated position, the expansion device 48 (e.g., the bellows 56), which may be coupled with the first actuator 22 via a linkage 134, may be in an expanded configuration such that the expanders 16 (not shown in FIG. 29A) are not expanded. As depicted in FIG. 29A, the first actuator 22 covers the second actuator 24 when in an unactuated position and thus, may prevent the second actuator 24 from being accessed to move the blade 80 without actuating the first actuator 22 and expanding the expanders 16.

FIG. 29B schematically depicts the first actuator 22 in an actuated position, similar to as discussed with respect to FIG. 28B. When in the actuated position, the expansion device 10 has been rotated distally into the slot 132, the linkage 134 has been advanced proximally, and in response to movement of the linkage 134 and the first actuator 22, the expansion device 48 may be in a compressed or contracted position to expand the expanders 16. Further, in response to actuating the first actuator 22, the second actuator 24 becomes accessible to a user, such that a user may adjust the second actuator 24 to move the blade 80 between one or more protected positions and one or more exposed or cutting positions.

FIG. 30 depicts a schematic perspective view of an illustrative configuration of the device 10, where the first actuator 22 may be a button and the second actuator 24 may be a slider positioned and adjustable at a location distal of the first actuator 22. In some examples, the configuration of the first actuator 22 and the second actuator 24 depicted in FIG. 30 may be the same as or similar to as depicted FIG. 20, but other suitable configurations are contemplated. A portion of the shaft 18 has been removed to depict the blade shaft 62 and the blade 80.

The first actuator 22 depicted in FIG. 30 may be in a pressed down and actuated position. As depicted in FIG. 30, the second actuator 24 has been moved proximally from a distal-most position to adjust a position of the blade 80.

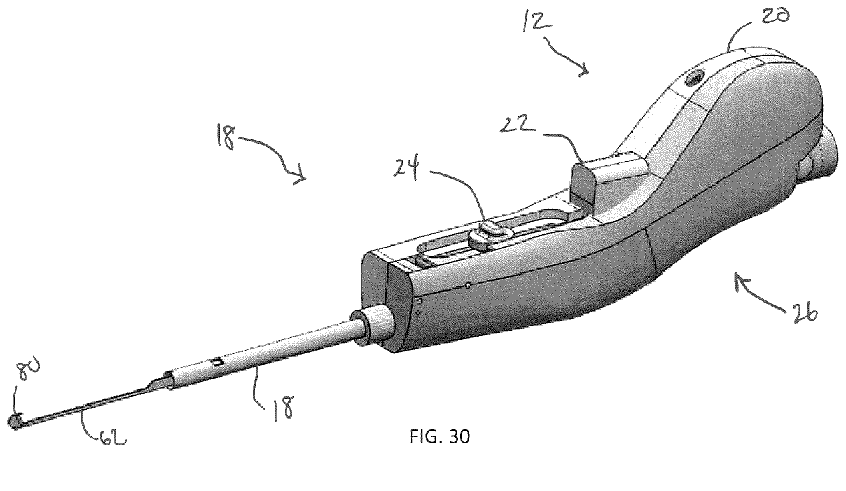

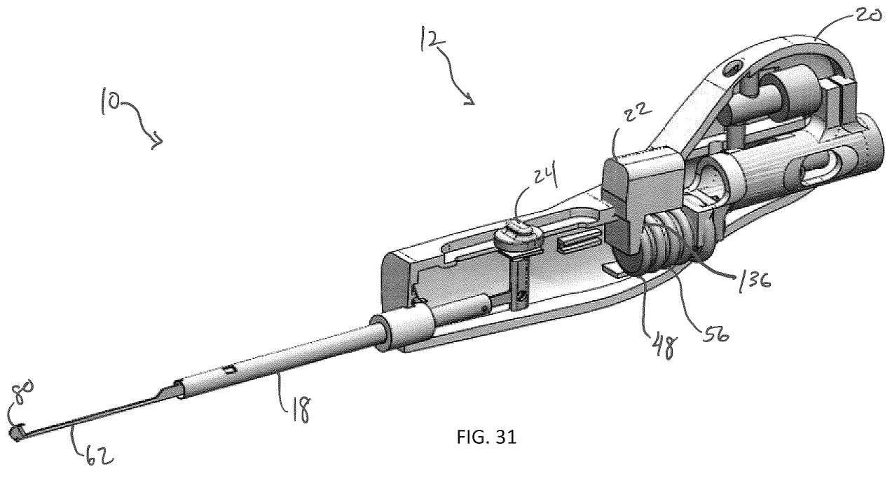

FIG. 31 schematically depicts the first actuator 22 in an actuated position, where a ramped surface 136 of or coupled with the first actuator 22 may engage the expansion device 48 (e.g., the bellows 56) to increasingly compress the expansion device 48 as the first actuator 22 pressed further down. Further and as discussed, the second actuator 24 be moved longitudinally relative to the housing 20 to adjust or move the blade 80 between one or more protected positions and one or more exposed or cutting positions. Although not necessarily depicted in FIG. 31, the second actuator 24 may not be movable until the first actuator 22 is in an actuated position, but this is not required.

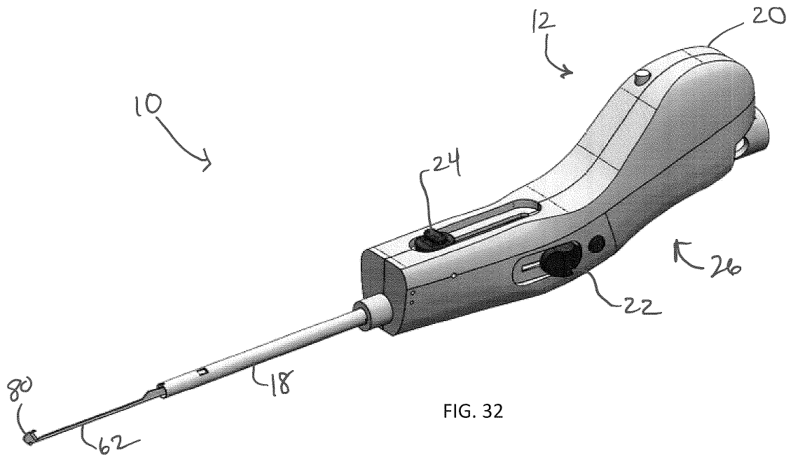

FIG. 32 depicts a schematic perspective view of an illustrative configuration of the device 10, where the first actuator 22 may be a slider on a lateral side of the handle 12 and the second actuator 24 may be a slider positioned and adjustable at a location along a top of the handle 12. A portion of the shaft 18 has been removed to depict the blade shaft 62 and the blade 80.

The first actuator 22 depicted in FIG. 32 may be slid to or otherwise adjusted to a proximal, actuated position. As depicted in FIG. 32, the second actuator 24 may be in a distal-most position such that the blade 80 may be in a protected position.

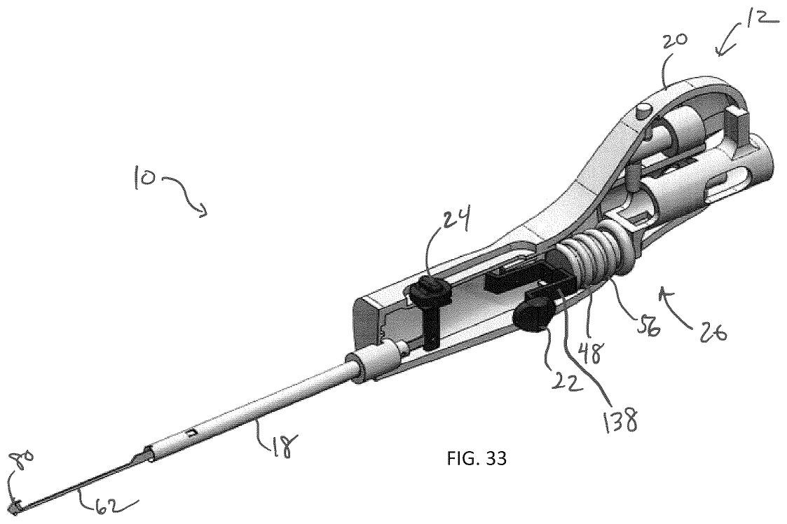

FIG. 33 schematically depicts the first actuator 22 in an actuated position, where an extension 138 of or coupled with the first actuator 22 may engage the expansion device 48 (e.g., the bellows 56) to compress the expansion device 48 as the first actuator 22 is moved proximally. Further and as discussed, the second actuator 24 may be moved longitudinally relative to the housing 20 to adjust or move the blade 80 between one or more protected positions and one or more exposed or cutting positions. Although not necessarily depicted in FIG. 33, the second actuator 24 may not be movable until the first actuator 22 is in an actuated position, but this is not required.

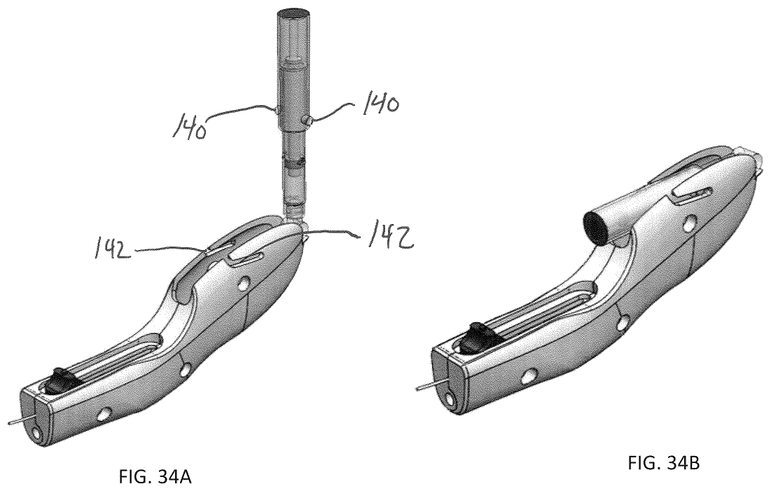

FIGS. 34A and 34B schematically depict perspective views of an illustrative configuration of the handle 12 of the device 10. The handle 12 depicted in FIGS. 34A and 34B may include the first actuator 22 in a lever configuration and the second actuator 24 in a slider configuration positioned and adjustable at a location along a top of the handle 12. The first actuator 22 in the configuration depicted in FIGS. 34A and 34B may be configured to rotate and depress in a “pen-click” manner to activate or expand the expanders 16 (not shown in FIGS. 34A and 34B).

The first actuator 22 depicted in FIG. 34A has been adjusted to an upright position and rotated such that protrusions 140 may be aligned with the slots 142 when the first actuator 22 is adjusted to a horizontal, actuated position as depicted in FIG. 34B. In some examples, when the first actuator 22 is in the upright position, the first actuator 22 may be depressed (e.g., similar to how a pen may be actuated) to actuate and expand the expanders 16. The first actuator 22 may then be rotated to a horizontal position and the protrusion 140 may engage the slots 142 to maintain the first actuator 22 in an actuated configuration. Although the not required, the process may be repeated to adjust the first actuator 22 to an unactuated position. Further, the second actuator 24 may be moved longitudinally relative to the housing 20 to adjust or move the blade 80 between one or more protected positions and one or more exposed or cutting positions. The second actuator 24 may not be movable until the first actuator 22 is in an actuated position, but this is not required.

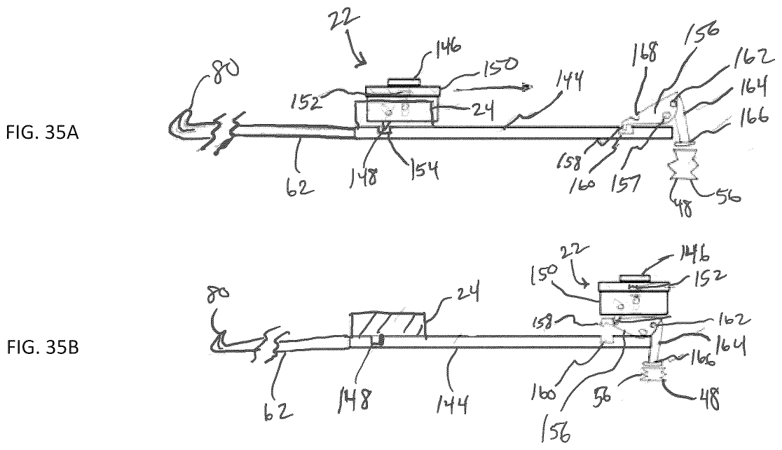

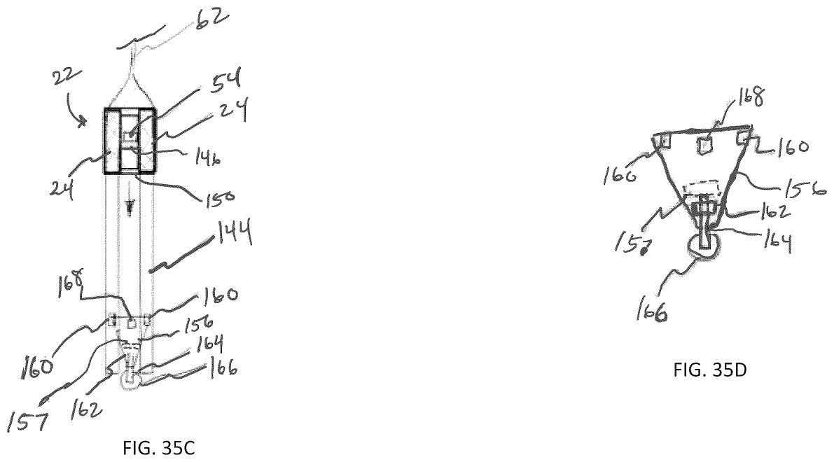

FIGS. 35A-35D are schematic side, side, top, and magnified top views, respectively, of an illustrative configuration of a portion of the device 10. As depicted in FIGS. 35A and 35B, the first actuator 22 may be released from a first position and may travel to a second position at which the first actuator 22 may compress the expansion device 48 (e.g., the bellows 56) to expand the expanders 16 (not show in FIGS. 35A-35D).

As depicted in FIG. 35A, the first actuator 22 may be located at the second actuator 24, where the second actuator 24 may be adjusted along the slide 144. The first actuator 22 may include a button 146 configured to release a tooth 148 from the slide 144 that maintains the first actuator 22 at the second actuator 24 in an unactuated position. Further, the first actuator 22 may include a base 146, where compressing the button 146 against a force applied to the button 146 by a spring 152 and relative to the base 146 may disengage the tooth 148 from a notch 154 of the slide 144 to allow the first actuator 22 to move relative to the slide 144.

When the first actuator 22 is adjusted proximally to a location proximate the expansion device 48, as depicted in FIG. 35B, the base 150 may engage a linkage 156 configured to pivot about a pivot component 157. A first end of the linkage 156 may include a tooth 158 configured to engage a notch 160 in the slide 144. A second end of the linkage 156 may be coupled at a pivot component 162 to a rod 164 with a compression plate 166 configured to engage the expansion device 48. Further, the linkage may include a notch 169 for engaging the tooth 148

FIG. 35C depicts a top view of the configuration depicted in FIG. 35A with the first actuator 22 positioned at the second actuator 24. FIG. 35D depicts a magnified top view of linkage 156, the rod 164, and the compression plate 166 a proximal end of the configuration depicted in FIG. 35C.

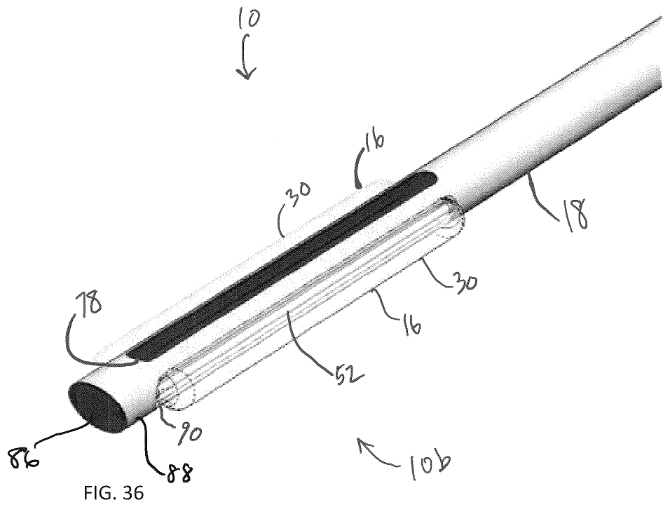

FIG. 36 depicts a schematic perspective view of a portion of a distal end of an illustrative configuration of the device 10. As depicted in FIG. 36, the distal end 10b may include the shaft 18, the opening 78 for receiving an exposed blade 80, two laterally expanding expanders 16 (e.g., balloons 30), and the tube 52 extending to or through the expanders 16.

The portion of the distal end 10b of the device 10 depicted in FIG. 36 may be formed from any suitable materials. In some examples, the shaft 18 may include the cover component 88, which may be formed from a hypotube and may have recess surfaces 90 (e.g., dimpled pockets or cradles) configured to receive the expanders 16 as the expanders 16 inflate or otherwise expand.

The hypotube forming at least a portion of an outer surface of the shaft 18 may be formed from any suitable material. In some examples, the hypotube may be formed from a stainless steel, but other suitable materials are contemplated.

In some examples, the shaft 18 may include the base component 86 extending at least partially along an interior of the cover component 88. In some configurations, the base component 86 may include or at least partially define the guideway for the blade 80 (not shown in FIG. 36).

The base component 86 may be formed from any suitable material. For example, the base component 86 extending within the cover component 88 may be formed from a metal, a polymer, a plastic, and/or one or more other suitable materials.

The opening 78 in the shaft 18 may be formed in any suitable manner. In some examples, the opening 78 may be laser cut into the hypotube or other suitable structure forming the shaft 18, but other suitable techniques for forming the opening 78 are contemplated.

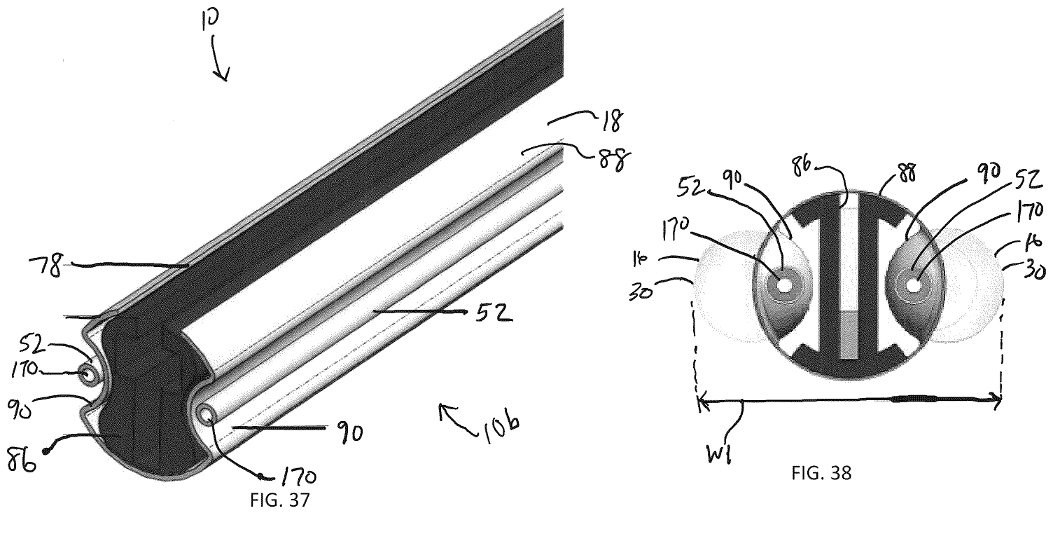

FIG. 37 depicts a schematic perspective view of a portion of the distal end 10b of an illustrative configuration of the device 10, where the portion is taken along the opening 78. As depicted in FIG. 37, the tubes 52 may include a lumen 170 configured to deliver fluid (e.g., saline and/or other suitable fluid) to the expanders 16.

The tubes 52 may be formed from any suitable material. In some examples, the tubes 52 may be formed from nylon, but other suitable materials are contemplated.

FIG. 38 depicts a schematic cross-section view of the distal end 10b of the illustrative configuration of the device 10, where the shaft 18 may be at least partially formed with the cover component 88 and the base component 86 extending within the cover component 88. The cross-section of the distal end 10b of the device 10 depicted in FIG. 38 may have any suitable width W1. Example suitable widths W1 include, but are not limited to, widths in a range between 6 mm and 10 mm, but the width W1 may be less than 6 mm or greater than 10 mm. In one example, the width W1 may be about 8 mm. Other suitable configurations are contemplated.

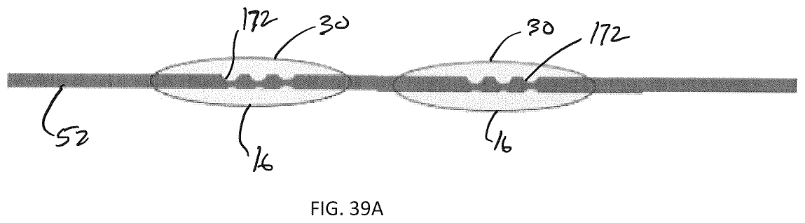

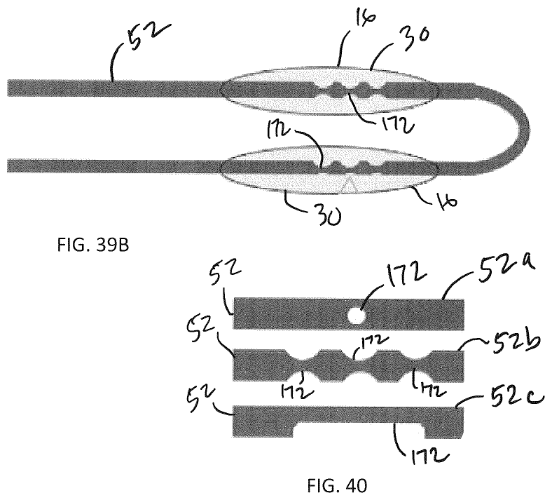

FIGS. 39A and 39B depict views of a single tube 52 with two expanders 16 in the form of balloons 30 or other suitable inflatable components in fluid communication with a lumen of the tube 52 through fluid openings 172. FIG. 39A depicts the single tube 52 in an unfolded configuration and FIG. 39B depicts the single tube in a folded configuration, which may be coupled to the shaft 18 of the device 10. The tube 52 may include a single lumen or more than one lumen.

Fluid may pass from one or both ends of the tube 52 through the lumen of the tube 52 to the fluid openings 172 and into the balloons 30 when the balloons 30 are to be inflated or expanded. When the balloons 30 are to be deflated, the fluid may be pulled from the balloons 30 through the fluid openings 172, into the lumen of the tube 52, and into a fluid a reservoir of the device 10 or into one or more other suitable location.

FIG. 40 schematically depicts configurations of the fluid openings 172 in the tubing 52, which may be located at the balloons 30 and/or other suitable inflatable components. A first configuration of the tube 52a may include a single circular opening configured to pass fluid to and from the balloons 30 and/or other suitable inflatable components. A second configuration of the tube 52b may include multiple circular fluid openings 172 on opposing or non-opposing sides of a tube that are configured to pass fluid to and from the balloons 30 and/or other suitable inflatable components. A third configuration of the tube 52c may include a single rectangular or elongate fluid opening 172 that is configured to pass fluid to and from the balloons 30 and/or other suitable inflatable components.

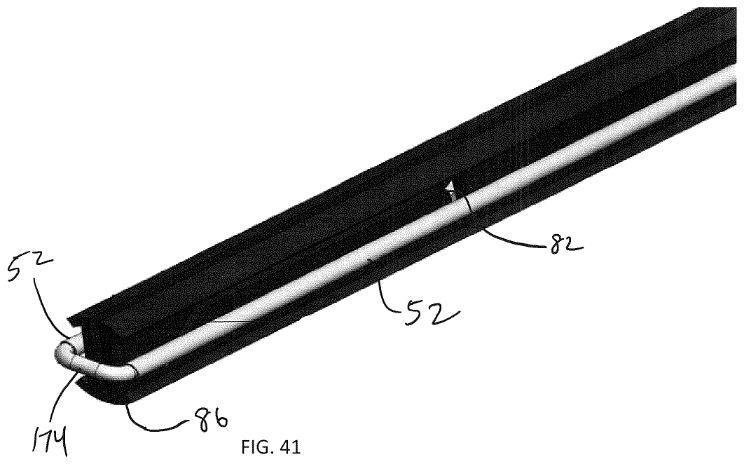

FIG. 41 schematically depicts a perspective view of an illustrative configuration of the base component 86 of the shaft 18, with two tubes 52 extending along the base component 86. In some examples, the base component 86 may entirely or at least partially define the guideway 82 to facilitate adjusting the blade (not shown in FIG. 41) between a protected position and a cutting position as the blade 80 travels longitudinally along the base component 86.

The two tubes 52 may be coupled to one another. In some examples, coupling the tubes 52 to one another may facilitate stabilizing a position of the tubes 52 along the base component 86. In some examples, coupling the tubes 52 to one another may fluidly couple the tubes 52 such that a single lumen may extend through both tubes 52.

The tubes 52 may be coupled to one another in any suitable manner. For example, the tubes 52 may be coupled to one another by engaging a tube connector 174 with the distal ends of the tubes 52, as depicted in FIG. 41, but other suitable coupling techniques are contemplated. The tube connector 174 may define one or more lumens or may be configured to prevent fluid from passing therethrough.

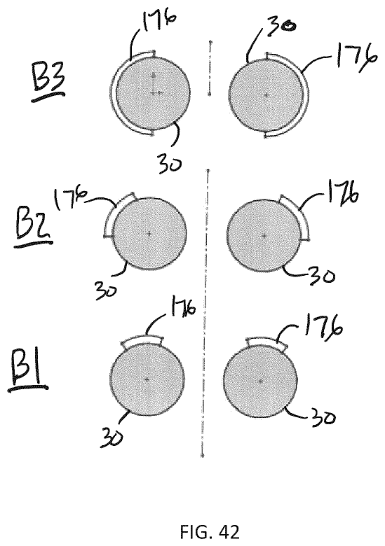

FIG. 42 depicts cross-sections of three sets of balloons 30 with micro-texture surfaces 176 thereon. Any suitable micro-texture surfaces may be used with the balloons 30.

The micro-texture surfaces 176 may be applied to any suitable portion of the outer circumference of the balloons 30 and/or other suitable expanders 16. For example, the micro-texture surfaces 176 may be applied to a portion of the balloons 30 that may engage body tissue of the patient. In some examples, a first set B1 of the balloons 30 may include micro-texture surfaces 176 facing substantially upward. In some examples, a second set B2 of the balloons 30 may include micro-texture surfaces 176 facing upward and and laterally, where the micro-texture surfaces 176 in the second set B2 of the balloons 30 may extend over a greater surface area of the balloons 30 than the micro-texture surfaces of the first set B1 of the balloons 30. In some examples, a third set B3 of the balloons 30 may include micro-texture surfaces 176 facing upward, downward, and laterally, where the micro-texture surfaces 176 in the third set B3 of the balloons 30 may extend over a greater surface area of the balloons 30 than the micro-texture surfaces of the second set B2 of the balloons 30. Other suitable applications of materials with or forming the micro-texture surfaces 176 to the balloons 30 are contemplated.

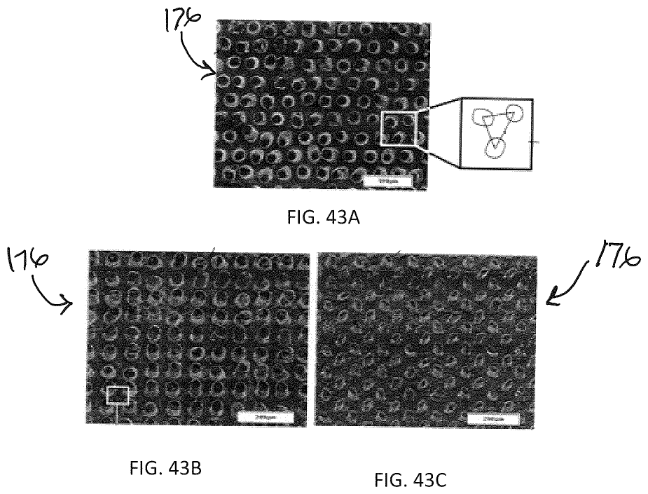

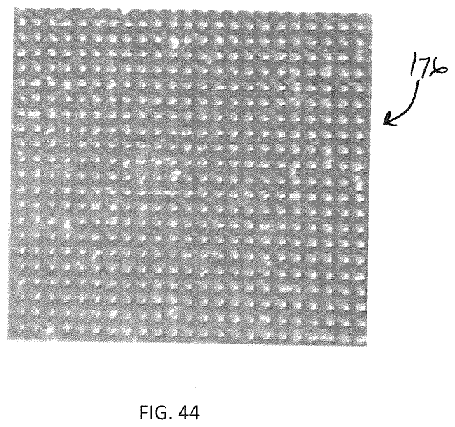

FIGS. 43A-43C schematically depict top views of different portions of material each with different micro-texture surfaces 176. FIG. 44 schematically depicts a top view of a material with a micro-texture surface 176.

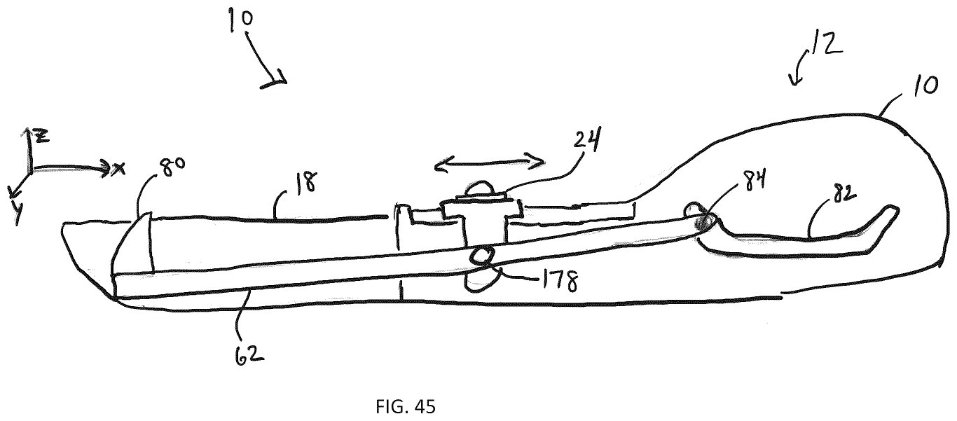

FIG. 45 depicts a schematic side view of an illustrative configuration of a device 10 for cutting soft tissue, where the guideway 82 for controlling a position of the blade 80 relative to the shaft 18 may be positioned in the handle 12 of the device 10. Portions of the device 10 have been omitted.

A distal end of the blade shaft 62 may include or be coupled with the blade 80 and a proximal end of the blade shaft 62 may include or be coupled with the pin 84 or other suitable protrusion configured to engage and travel along the guideway 82. In some examples, the second actuator 24 may be coupled with the blade shaft 62 at a pivot component 178 located between the distal end and the proximal end of the blade shaft 62.

When the guideway 82 is positioned proximal of the pivot component 178 coupling the second actuator 24 and the blade shaft 62, the guideway 82 may be inverted relative to when the guideway 82 is positioned distal of a coupling between the second actuator 24 and the blade shaft 62. In operation, as the second actuator 24 is adjusted longitudinally (e.g., proximally and/or distally in a direction of an x-axis), the pin 84 or other suitable component may travel along the guideway 82, causing the blade shaft 62 to pivot about the pivot component 178 and adjust the blade 80 between one or more protected positions and one or more exposed or cutting positions (e.g., in a z-axis).

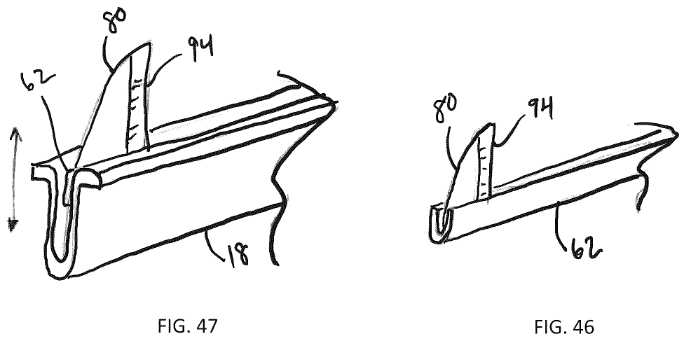

FIG. 46 schematically depicts a perspective view of an illustrative configuration of the blade shaft 62 coupled with the blade 80. The blade shaft 62 may be formed from steel or other suitable material to form a rugged blade shaft 62 configured to encounter resistance forces in the z-axis direction depicted in FIG. 45. The blade 80 may be coupled to the blade shaft 62 in any suitable manner including, but not limited to, via a welding technique, such as spot welding or other suitable welding technique. Coupling the blade 80 to the blade shaft 62 without a pin or other similar component may reduce a width profile of the blade 80 and blade shaft 62. Further, the blade shaft 62 may be configured without a flange to facilitate vertically translating (e.g., in the z-axis depicted in FIG. 45) the blade 80.

FIG. 47 schematically depicts a perspective view of an illustrative configuration of the blade 80 and blade shaft 62 combination depicted in FIG. 46 within the shaft 18. The shaft 18 may be formed from steel or other suitable rugged material. In some examples, the shaft 18 and the blade shaft 62 and blade 80 combination depicted in FIG. 47 may be utilized in the configuration of the device 10 depicted in FIG. 45 and/or other suitable configurations of the device 10.

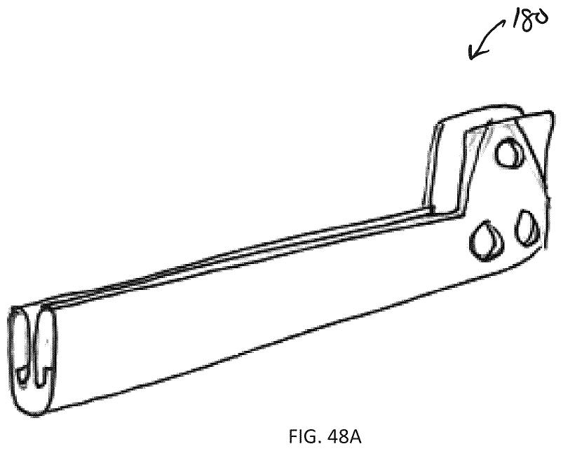

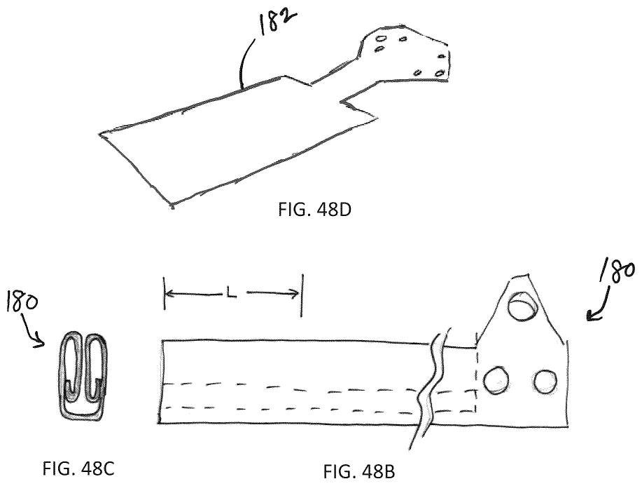

FIGS. 48A-48D schematically depict views of a channel 180 for receiving the blade 80 (not shown in FIGS. 48A-48D). The channel 180 may be configured as at least a part of the shaft 18 and/or a part of the blade shaft 62, but other suitable configurations are contemplated.

FIG. 48A schematically depicts a perspective view of an illustrative configuration of the channel 180. FIG. 48B schematically depicts a side view of the channel 180 depicted in FIG. 48A. FIG. 48C schematically depicts a cross-section view of the channel 180 depicted in FIG. 48A. FIG. 48D schematically depicts a sheet of material 182 that has been cut to a shape that may be used to form the channel 180. In some examples, the cut sheet of material 182 may be formed from a sheet of metal material cut to the desired dimensions, which may then be folded or bent or machined using one or more other suitable technique to the desired shape of the channel 180.

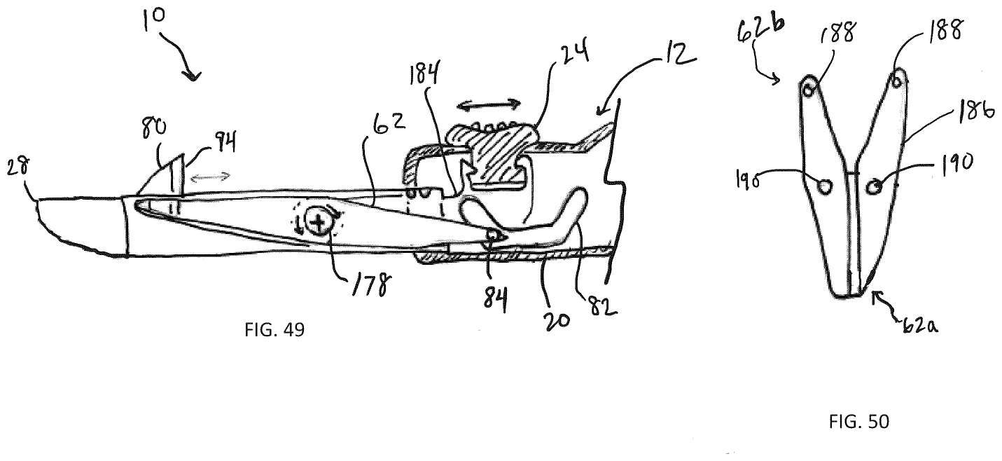

FIG. 49 depicts a schematic side view of an illustrative configuration of a device 10 for cutting soft tissue, where the guideway 82 for controlling a position of the blade 80 relative to the shaft 18 may be positioned in the handle 12 of the device 10. Portions of the device 10 have been omitted and/or shown in cross-section.

A distal end of the blade shaft 62 may include or be coupled with the blade 80 and a proximal end of the blade shaft 62 may include or be coupled with the pin 84 or other suitable protrusion configured to engage and travel along the guideway 82. In some examples, the second actuator 24 may be coupled with the blade shaft 62 via a connector 184 at the pivot component 178 located between the distal end and the proximal end of the blade shaft 62. In some examples, the connector 184 may be utilized to facilitate positioning the pivot component 178 close to the blade 80 (e.g., closer to the blade 80 relative to a position of the pivot component 178 in the configuration of the device 10 depicted in FIG. 45), which may facilitate improved control over a position of the blade relative to when the pivot component 178 at the coupling between the second actuator 24 and the blade 62 is farther from the blade 80.

When the guideway 82 is positioned proximal of the pivot component 178 coupling the second actuator 24 and the blade shaft 62, the guideway 82 may be inverted relative to when the guideway 82 is positioned distal of a coupling between the second actuator 24 and the blade shaft 62. In operation, as the second actuator 24 is adjusted longitudinally (e.g., proximally and/or distally), the pin 84 or other suitable component may travel along the guideway 82, causing the blade shaft 62 to pivot about the pivot component 178 and adjust the blade 80 between one or more protected positions and one or more exposed or cutting positions (e.g., as depicted in FIG. 49).

FIG. 50 schematically depicts a sheet of material 186 that has been cut to a shape that may be used to form the configuration of the blade shaft 62 depicted in FIG. 49. In some examples, the cut sheet of material 186 may be formed from a sheet of metal material cut to the desired dimensions, which may then be folded or bent or machined using one or more other suitable technique to the desired shape of the blade shaft 62. The blade 80 may be affixed to a first end 62a of the blade shaft 62. A second end 62b of the blade shaft 62 may include openings 188 configured to receive the pivot component or pin 84. Openings 190 may be located between the first end 62a and the second 62b of the blade shaft 62 and may be configured to received the pivot component 178. Alternatively or additionally, one or more of the openings 188, 190 may be pivot components.

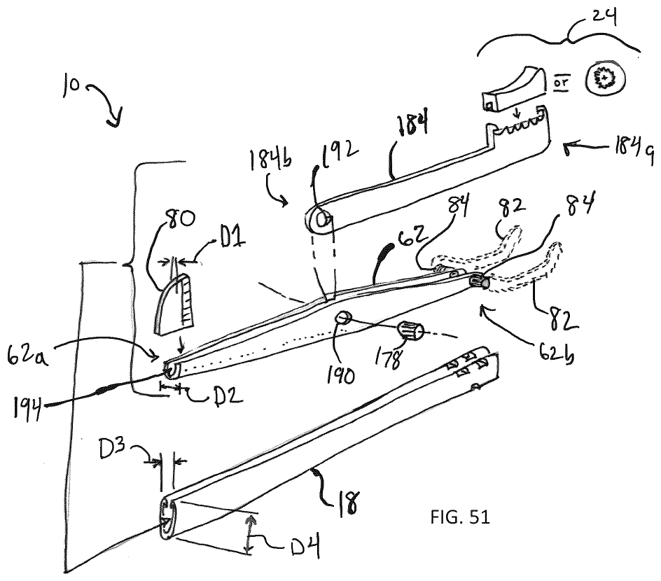

FIG. 51 schematically depicts an exploded view of the device 10 depicted in FIG. 49, with the housing 20 omitted. As depicted in FIG. 51, the second actuator 24 may couple with or otherwise engage a first end 184a (e.g., a proximal end) of the connector 184. The second actuator 24 may have any suitable configuration including, but not limited to, a slider configuration, a gear wheel configuration, and/or other suitable configuration. A second end 184b (e.g., a distal end) of the connector 184 may be inserted into a slot 194 of the blade shaft 62 and the pivot component 178 may be inserted through one or more openings 190 of the blade shaft 62 between the first end 62a and the second end 62b and through the opening 192 in the connector 184 such that the blade shaft 62 may pivot relative to the connector 184. The first end 62a of the blade shaft 62 may be coupled with the blade 80 (e.g., via a weld connection and/or one or more other suitable couplings) and the second end 62b of the blade shaft 62 may include the pin 84 configured to traverse the guideway(s) 82. The blade shaft 62 may then be inserted into the shaft 18. Other suitable configurations of the device 10 are contemplated.

The components of the device 10 depicted in FIGS. 49 and 51 may have any suitable configurations and/or dimensions. In some examples, the blade 80 may have a width dimension D1 of about 0.508 mm (0.020 inches), the blade shaft 62 may have a width dimension D2 of about 2.032 mm (0.080 inches), the shaft 18 may have a width dimension D3 of about 3.556 mm (0.140 inches), and the shaft 18 may have a height dimension D4 of less than about 5 mm (0.19685 inches). Further, in some examples, the blade shaft 62 and/or the shaft 18 may be formed a sheet of material having a thickness of about 0.762 mm (0.030 inches). In some configurations, the handle 12 may be formed from a plastic material. Other suitable configurations and/or dimension of the components of the device 10 are contemplated.

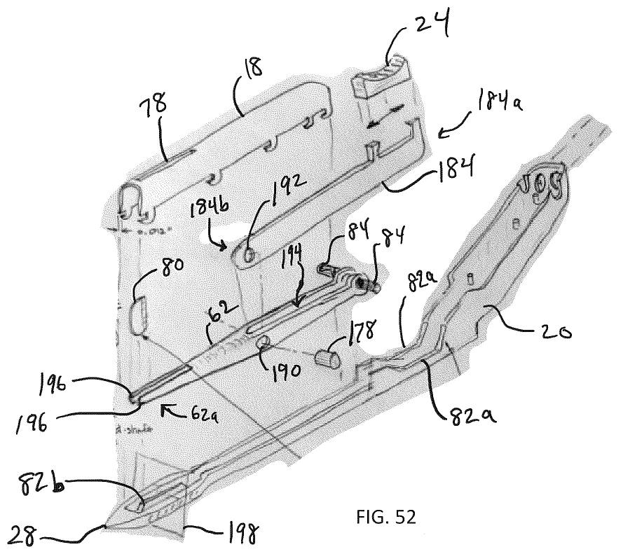

FIG. 52 schematically depicts an exploded view of an illustrative configuration the device 10, with portions of the device 10 omitted. As depicted in FIG. 52, the second actuator 24 may couple with or otherwise engage the first end 184a (e.g., a proximal end) of the connector 184. The second actuator 24 may have any suitable configuration including, but not limited to, a slider configuration, a gear wheel configuration, and/or other suitable configuration. The second end 184b (e.g., a distal end) of the connector 184 may be inserted into the slot 194 of the blade shaft 62 and the pivot component 178 may be inserted through one or more openings 190 of the blade shaft 62 between the first end 62a and the second end 62b and through the opening 192 in the connector 184 such that the blade shaft 62 may pivot relative to the connector 184. The first end 62a of the blade shaft 62 may include two laterally spaced prongs 196 configured to couple with the blade 80 (e.g., via a weld connection and/or one or more other suitable couplings) and the second end 62b of the blade shaft 62 may include the pin 84 configured to traverse a first guideway 82a (e.g., a ramp) in or defined by a proximal portion of the housing 20 and/or other suitable component. The housing 20 may include a distal portion configured to extend along the blade shaft 62 and may include a second guideway 82b (e.g., a ramp) configured to engage the blade 80. The prongs 196 of the blade shaft 62 may be spaced from one another so as to be configured to receive the second guideway 82b until the blade 80 engages the second guideway 82b to facilitate raising the blade 80 to an exposed or cutting position, but this is not required. In some cases, the distal portion of the housing 20 may include a tapered tip 28 configured to facilitate inserting the device 10 into tissue of a patient. The shaft 18 with the opening 78 configured to receive the blade 80 when the blade 80 is in an exposed or cutting position may be applied to the blade shaft 62 and/or the housing 20 and may at least partially cover a portion of the blade shaft 62. Other suitable configurations of the device 10 are contemplated.

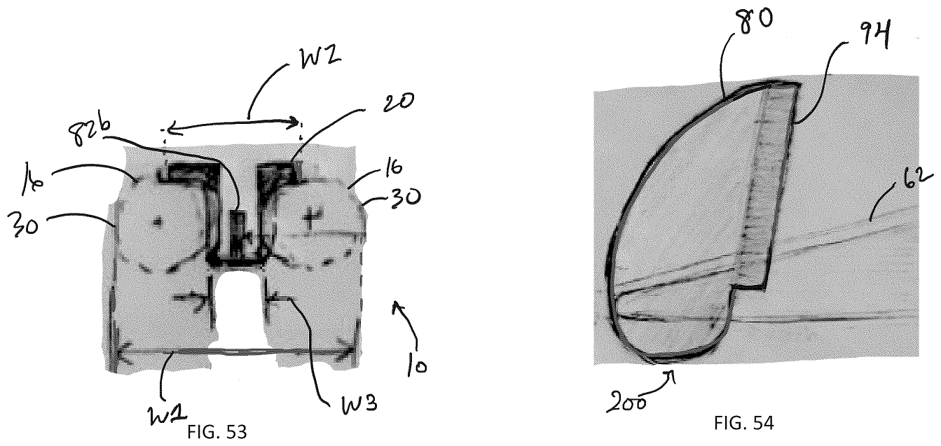

FIG. 53 schematically depicts a cross-section view of the distal portion of the housing 20 depicted in FIG. 52, taken at box 198 and with expanders 16 (e.g., balloons 30) added-in. The housing 20 and expanders 16 depicted in FIG. 53 may have any suitable configurations and dimensions. In some examples, the width W1 of the device 10 at the cross-section may be about 8 mm, a width W2 at a top of the housing 20 in the cross-section may be about 5 mm, a width W3 of a channel of the housing 20 configured to receive the blade shaft 62 and including the second guideway 82b may be about 4 mm, and a width of the second guideway 82b may be about 0.508 mm (0.020 inches) wide. Other suitable configurations and dimensions are contemplated.

FIG. 54 schematically depicts the blade 80 positioned at the blade shaft 62. In some examples, a bottom surface 200 of the blade 80 may be configured to engage the second guideway 82b as the blade 80 travels longitudinally relative to the second guideway 82b.

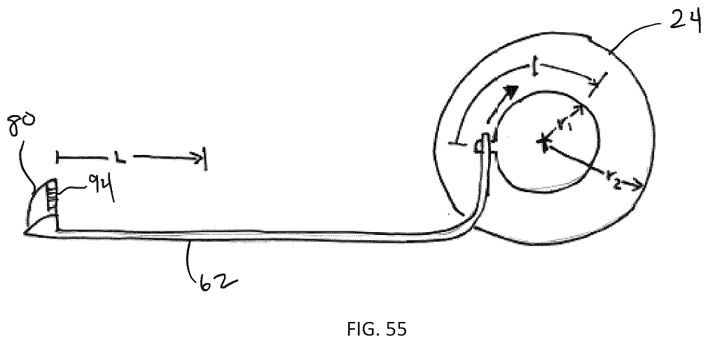

FIG. 55 schematically depicts a side view of an illustrative configuration of the second actuator 24 coupled with blade shaft 62 having a flexible configuration and a distal end with the blade 80 coupled thereto. The blade shaft 62 may be formed from a flexible metal, a flexible polymer, and/or other suitable material.

As depicted in FIG. 55 the second actuator 24 may have a wheel configuration, where the blade shaft 62 may be coupled with a portion of the second actuator 24 having a first radius r1 and a user may be configured to engage and rotate a portion of the second actuator 24 having a second radius r2, where the radius r2 is greater than radius r1 such that a distance, l, the second actuator 24 travels may be greater than a distance, L, the blade travels. Such a configuration of the second actuator 24 relative to the blade shaft 62 may facilitate a user making more precise adjustments of a position of the blade. IN some configurations, when a user is concerned about making long cuts, the second actuator 24 may be configured such that r1 is greater than r2. Other suitable configurations of the second actuator 24 and the blade shaft 62 are contemplated.

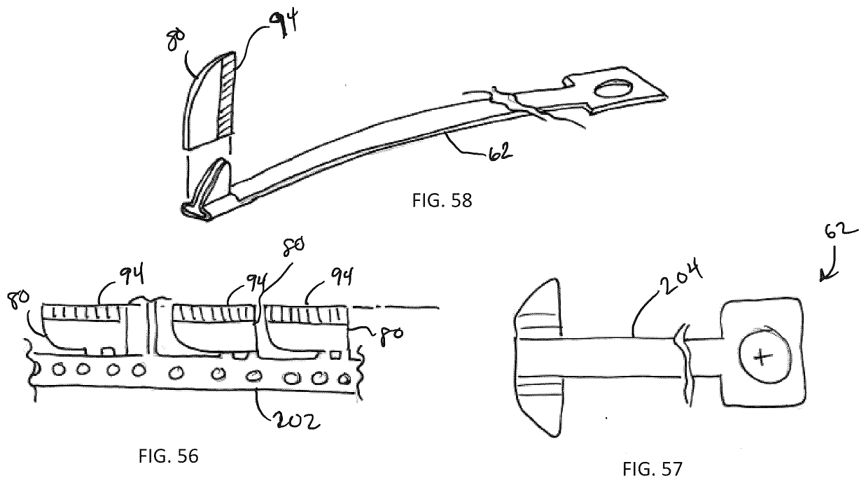

FIG. 56 schematically depicts a side view of blades 80 formed on a sheet of material. The blades 80 may be separated from the sheet of material 202 for coupling with the blade shaft 62.

FIG. 57 schematically depicts a top view of a cut out or stamped material 204 for an illustrative configuration of the blade shaft 62. The cut out or stamped material 204 may be bent, folded, or machined in one or more other suitable manners to form the blade shaft 62.

FIG. 58 schematically depicts a perspective view of an assembly of the blade 80 with the formed blade shaft 62, where the blade shaft 62 may be formed from the cut or stamped material 204 depicted in FIG. 57. As depicted in FIG. 58, the blade 80 may be inserted into a distal end of the blade shaft 62 and coupled with the blade shaft 62 in any suitable manner including, but not limited to, via a weld connection.

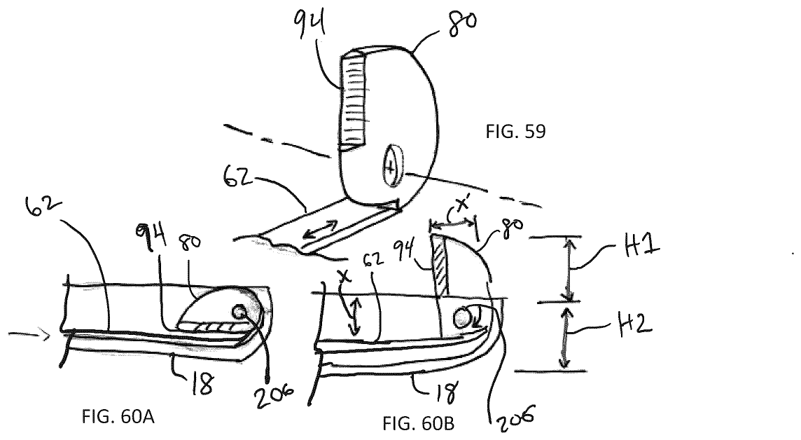

FIG. 59 schematically depicts a perspective view of an illustrative configuration of the blade 80 coupled with the blade shaft 62, where the blade shaft 62 may have a flexible configuration. When the blade shaft 62 is flexible and the blade 80 may be longitudinally fixed relative to longitudinal movement of the blade shaft 62, the blade 80 may be rotationally or pivotably adjusted between a protected position and an exposed or cutting position.

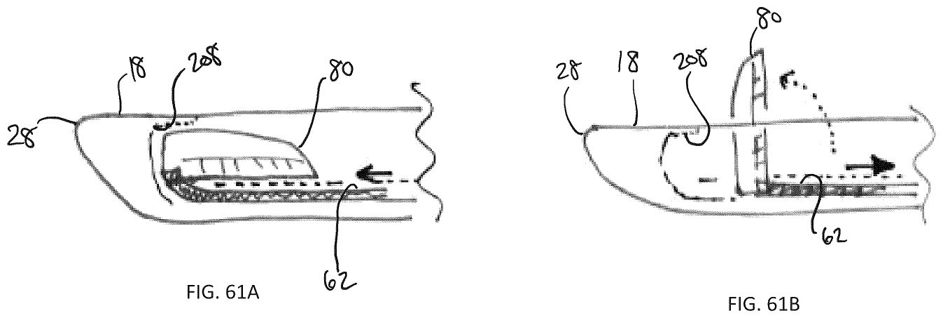

FIG. 60 schematically depicts the blade 80 and the blade shaft 82 within the shaft 18, where the blade 80 is in a protected position and may be configured to rotate or pivot about a fixed point 206 in the shaft 18. FIG. 61 schematically depicts the blade 80 and the blade shaft 82 with in the shaft 18, where the blade is in an exposed or cutting position after rotating or pivoting about the fixed point 206 in response to longitudinal adjustment of the blade shaft 62.

The components depicted in FIGS. 60 and 61 may have any suitable dimensions. For example, a distance x between a top surface of the blade shaft 62 and the top surface of the shaft 18 may be the same as or greater than a depth x′ of the blade 80. Further, as depicted in FIG. 61, a height H1 of the blade 60 exposed from the shaft 18 may be about 2.5 mm, which may be independent of a height H2 of the shaft 18.

FIGS. 61A and 61B schematically depict the blade 80 coupled with a distal end of the blade shaft 62, where the blade shaft 62 may be flexible and the blade 80 and blade shaft 62 may be positioned at the shaft 18. As depicted in FIGS. 61A and 61B, the shaft 18 may include a rounded portion 208 configured to engage the blade 80 and cause the blade 80 to rotate. FIG. 61A schematically depicts the flexible blade shaft 62 advanced forward or distally such that the blade 80 engages the rounded portion 208 is positioned in a protected position. FIG. 61B schematically depicts the flexible blade shaft 62 withdrawn proximally such that the blade 80 separates from the rounded surface 208 and is positioned in an exposed or cutting position. In some examples, the blade 80 may be biased to the exposed or cutting position.

FIG. 62 schematically depicts a perspective view of an illustrative configuration of the device 10, with components omitted for clarity. As depicted in FIG. 62, two expanders 16 in the form of paddles 210 may extend distally from the handle 12, where the paddles 210 may be adjustable in response to actuation of the first actuator 22 in the distal direction to move tissue away from the blade 80 (not shown in FIG. 62). In some examples, the paddles 210 may be biased to a delivery configuration and the first actuator 22, in a shuttle configuration under the housing 20 of the handle 12, may be longitudinally adjusted between proximal and distal positions to engage the expanders 16 adjust a width of the paddles 210. For example, as the first actuator 22 extends distally, the first actuator 22 may engage the paddles 210 or a cam for the paddles 210 to expand the paddles 210 and as the first actuator 22 extends proximally, the bias on the paddles 210 may narrow a width of the paddles 210.

FIGS. 63A-63D schematically depict views of an illustrative configuration of the expanders 16 with the paddle 210 configuration. FIG. 63A depicts a schematic top view of the expanders 16. In FIG. 63A, a portion of the expanders 16 to the right of the broken line may remain exterior of the patient (e.g., at the or proximate the handle 12 and/or the first actuator 22) and a portion of the expanders 16 to the left of the broken line may be inserted into the patient. A cam path 212 may lead to scissors (e.g., the paddles 210). FIG. 63B depicts a side view of an illustrative configuration of the expanders 16 with the paddle 210 configuration depicted in FIG. 63A.

FIG. 63C schematically depicts a cross-section view take along line A-A in FIG. 63A, where the paddles 210 are depicted as being configured to scissor. In some cases, the paddles 210 may be curved, but other suitable configurations are contemplated.

FIG. 63D schematically depicts a cross-section of the expanders 16 depicted in FIG. 63A, taken along line B-B. The vertical parallel portions may engage rails of or for the first actuator 22 to adjust along.

FIG. 64 schematically depicts a side view of a gear system coupling the blade shaft 62 and/or the connector 184 with the second actuator 24. In some examples, a proximal end of the blade shaft 62 and/or the connector 184 may include teeth configured to engage teeth of a first gear 216. The teeth of the first gear 216 may be configured to engage teeth of a second gear 218, where the second gear 218 may rotate with the second actuator 24 and have a radius that is less than a radius of the second actuator 24. In some configurations, the gear system may be configured relative to the second actuator 24 such that for every 2 units of linear length the second actuator 24 travels, the blade 80 (not shown in FIG. 64) travels 1 unit of linear length. Other suitable gear system configurations are contemplated.

FIG. 65 schematically depicts a side view of a linkage system coupling the blade shaft 62 and/or the connector 184 with the second actuator 24. In some examples, a proximal end of the blade shaft 62 and/or the connector 184 may be coupled with a first linkage 220. The first linkage 220 may be configured to engage the second linkage 222, where the second linkage 222 may adjust with the second actuator 24. In some configurations, the linkage system may be configured relative to the second actuator 24 such that for every 1 units of linear length the second actuator 24 travels, the blade 80 (not shown in FIG. 65) travels 1 unit of linear length. Other suitable gear system configurations are contemplated.

FIG. 66 schematically depicts a perspective view of an illustrative configuration of the device 10, with components omitted for clarity. As depicted in FIG. 66, the two expanders 16 may be in the form of paddles 210 and may extend distally from the handle 12, where the paddles 210 may be adjustable in response to actuation of the first actuator 22 to move tissue away from the blade 80 (not shown in FIG. 62). The first actuator 22 may have a trigger configuration such that the first actuator 22 may be clicked to position the paddles 210 in a first configuration (e.g., a probing position) and may be clicked a second time to position the paddles 210 in an expanded configuration. The second actuator 24 may be longitudinally adjusted to adjust a longitudinal position of the blade 80. In some configurations the paddles 210 may be molded and formed from a polymer, such as a polyetherimide, and/or other suitable material.

FIGS. 67A-67C schematically depict views of an illustrative configuration of the expanders 16 with the paddle 210 configuration. FIG. 67A schematically depicts a top view of the expanders 16, with the paddles 210 in an expanded configuration. When in the expanded configuration, the paddles 210 may have a width of about 8 mm, but other suitable dimensions are contemplated.

FIG. 67B schematically depicts a perspective view of the expanders 16 with the paddles 12 in an expanded configuration. When in the expanded configuration, a proximal portion of the expanders 16 may be configured to receive the first actuator 22 and/or a shuttle mechanism.

FIG. 67C schematically depicts a perspective view of the expanders 16 with the paddles 12 in a retracted or probing configuration. When in the probing configuration, the paddles 210 may have a height of about 5 mm and a width of about 3 mm, but other suitable configurations are contemplated. When the paddles 210 are in the probing position, the paddles 210 may be configured to facilitate delivery of the device 10 into the patient and/or exploration of the target area within the patient.

FIG. 68 schematically depicts a perspective view of an illustrative configuration of the device 10, with components omitted for clarity. As depicted in FIG. 68, the two expanders 16 may be in the form of paddles 210 and may extend distally from the handle 12, where the paddles 210 may be adjustable in response to actuation of the first actuator 22 and may result in longitudinal movement of a shuttle 224 to actuate the paddles 210 to move tissue away from the blade 80 (not shown in FIG. 68). The first actuator 22 may be any suitable type of actuator including, but not limited to, a slider, a knob, a lever, a trigger, a wheel (e.g., as depicted in FIG. 68), and/or other suitable type of actuator configured to advance and withdraw the shuttle 224. In some examples, gearing and/or linkages may extend from the first actuator 22 and/or the second actuator 24 (not shown in FIG. 68) to an undercarriage location at a base of the handle 12, where there may be space for linear, proximal-to-distal extending tracks, scissoring cams, rotational components, and/or other suitable components of the device 10.

The paddles 210 may have any suitable configuration. In some examples, the paddles 210 may be separate components or components coupled together. In one example configuration, the paddles 210 may be stamped from metal and configured to adjust in a scissors manner in response to actuation of the first actuator 22 and/or the shuttle 224. In another example, the paddles 210 may be molded from a polymer, such as a polyetherimide, and configured to be adjusted in a folding manner, where the paddles 210 may be folded for insertion and separated or unfolded to move tissue away from an exposed blade 80. Other suitable configurations of the paddles 210 are contemplated.

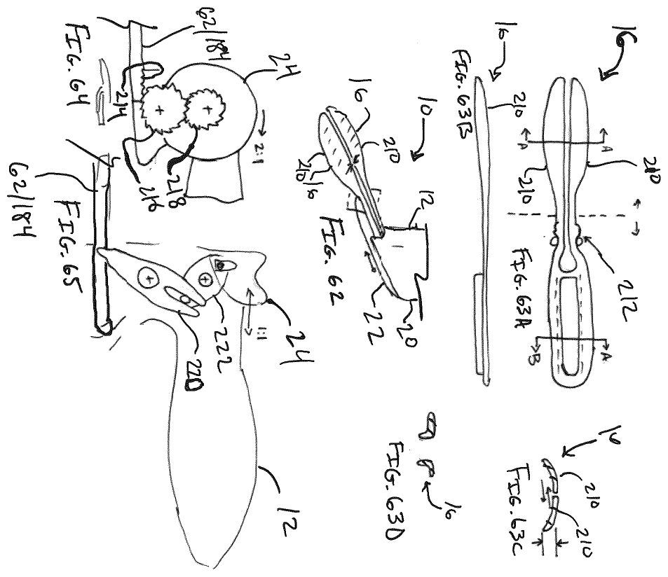

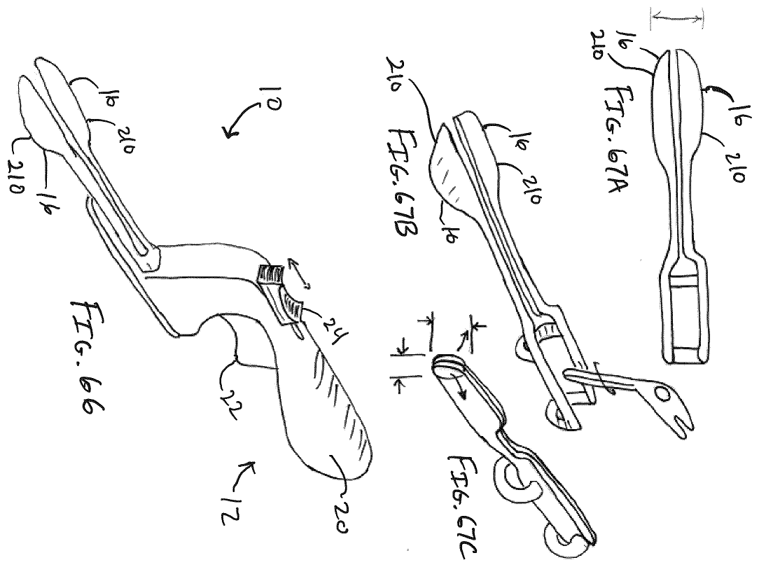

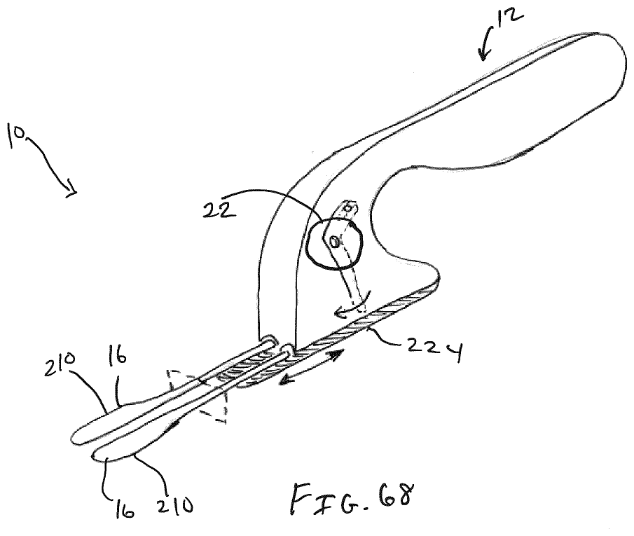

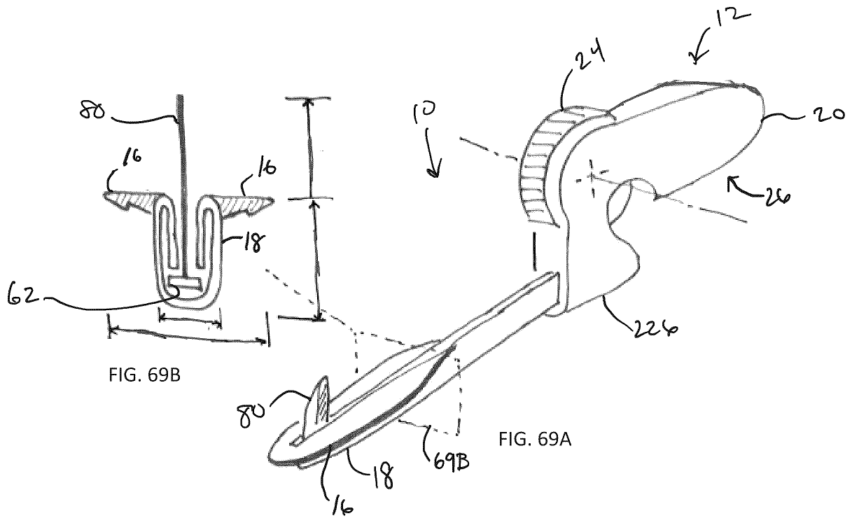

FIG. 69A schematically depicts a perspective view of an illustrative configuration of the device 10. As depicted in FIG. 69A, the shaft 18 extending distally from the handle 12 may include fixed expanders 16 proximate a path of the blade 80 in an exposed or cutting position. As the expanders 16 may have a fixed configuration relative to the shaft 18 and/or the handle 12, the expanders 16 may move tissue away from a central portion of the shaft 18 as the shaft 18 is inserted into a patient.

The handle 12 may include the grip portion 26 raised up from a base 226 of the handle 12 such that the base 226 of the handle 12 may rest on a patient or other surface during a procedure while allowing a user to grasp the handle 12. Such a configuration of the handle 12 may promote stability and natural upward pressure.

Further, the second actuator 24 may have a wheel configuration and may be configured to adjust the blade 80 one unit of linear length for each two units of linear length the second actuator 24 is adjusted, but other suitable configurations are contemplated. Further, the configuration of the device 10 may be stiff and rugged.

FIG. 69B depict schematic cross-section view take along rectangle 69B in FIG. 69A. As depicted in FIG. 69B, the blade 80 may extend from and/or may be coupled with the blade shaft 62 and extend within the shaft 18. The fixed expanders 16 may extend laterally outward from a location at or proximate a top of the shaft 18, but other suitable configurations are contemplated.

The components of the device 10 may have a suitable configurations and/or dimensions. For example, a width of the fixed guards may be about 5 mm, a width of the shaft 18 may be about 2 mm, a height of the shaft may be about 3 mm, and a height of the exposed portion of the blade 80 may be about 2.5 mm, but other suitable configurations and/or dimensions are contemplated.

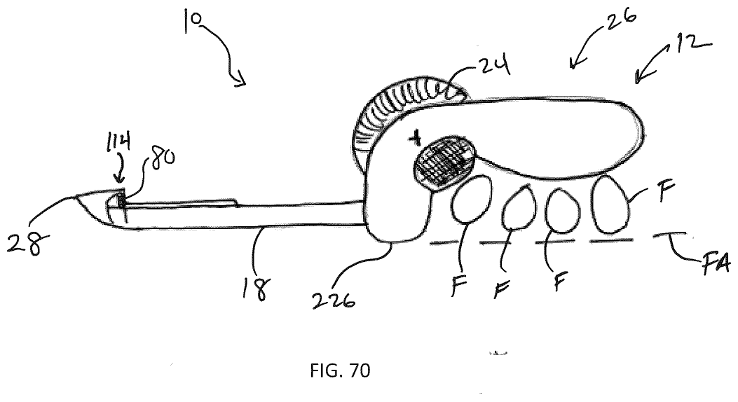

FIG. 70 schematically depicts a side view of an illustrative configuration of the device 10. As depicted in FIG. 70, the shaft 18 extending distally from the handle 12 may include the garage 114 configured to house the blade 80 during insertion and/or removal of the device 10 from a patient.

The handle 12 may include the grip portion 26 raised up from a base 226 of the handle 12 such that the base 226 of the handle 12 may rest on a patient or other surface during a procedure while allowing a user to grasp the handle 12. Further, when a user is gripping the handle 12 at the grip portion 26 with fingers F of a hand, the fingers F may rest or anchor on a forearm FA of the patient. Further, in some configurations, the base portion 226 may have a rounded or hemispherical configuration configured to rock on the patient's forearm FA to facilitate spatial tip adjustments through movement of the handle 12.

Further, the second actuator 24 may have a wheel configuration and may be configured to adjust the blade 80 one unit of linear length for each two units of linear length the second actuator 24 is adjusted. Other suitable ratios of relative movement are contemplated.

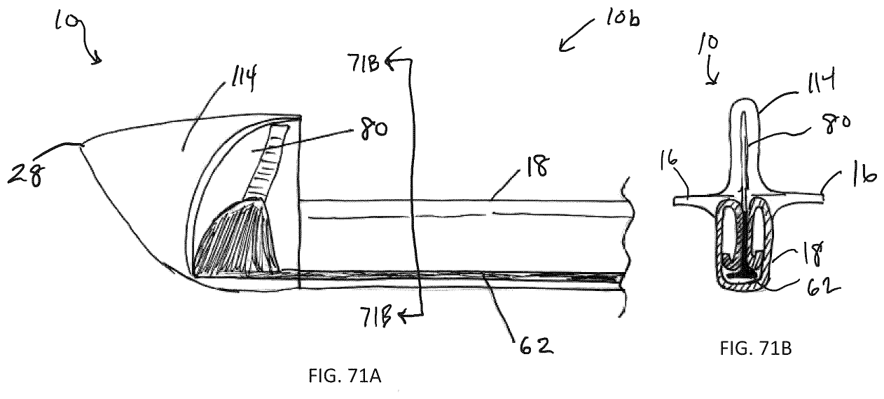

FIG. 71A schematically depicts a side view of a distal end 10b of the device 10, with portions of the device 10 omitted to show interior features of the device 10. As depicted in FIG. 71A, the shaft 18 may include or may define the garage 114 configured to receive the blade 80 coupled with the blade shaft 62, where the blade 80 is positioned within the garage 114. FIG. 71B schematically depicts the garage 114 with the blade therein and the blade shaft 62 below folded portions of the shaft 18 may facilitate preventing the blade shaft 62 and/or the blade 80 from rising above a desired amount. Further, the distal end 10b of the device 10 may include the expanders 16, which may have a fixed configuration, as depicted in FIG. 71B.

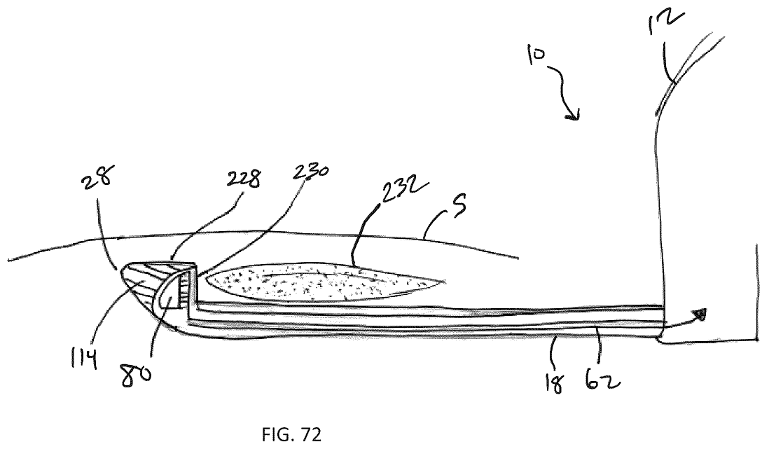

FIG. 72 schematically depicts a side view of the distal end 10b of the device 10, where the tip 28 may be utilized as a probing tip proximate soft tissue 232 under the skin S of the patient. The shaft 18 may include the garage 114 housing the blade 80 extending from the blade shaft 62. The shaft 18 may include the tip 28, which may be a distal tip probing surface, a superficial tip probing surface 228, and a proximal tip probing surface 230. As depicted in FIG. 72, the garage 114 and the blade 80 may be position distal of the soft tissue 232 and the probing surfaces 28, 228, 230, and/or other suitable surfaces may be utilized to probe within the patient to identify tissue to cut.

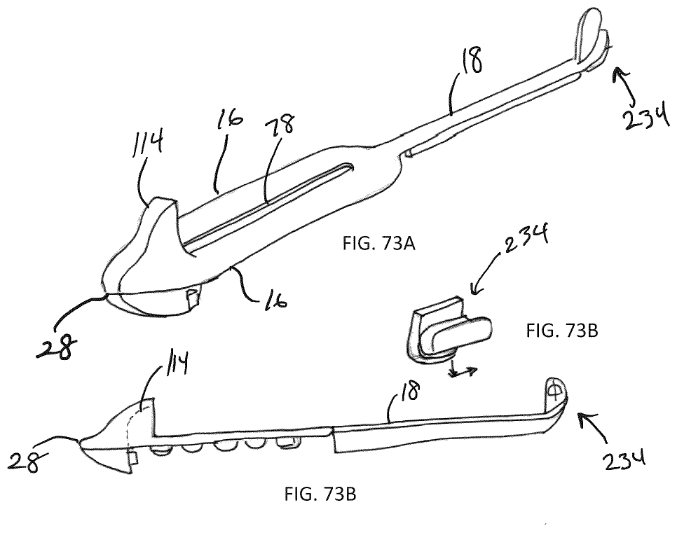

FIG. 73A schematically depicts a perspective view of an illustrative configuration of the shaft 18. The shaft 18 depicted in FIG. 73A may include the distal tip 28, the garage 114, fixed expanders 16, the opening 78 for receiving the blade 80 (not shown in FIG. 73A) in an exposed or cutting position and a proximal attachment component 234 configured to engage the handle 12 (not shown in FIG. 73A) of the device 10. FIG. 73B schematically depicts a side view of the shaft 12 depicted in FIG. 73A. FIG. 73C depicts an alternative configuration of the proximal attachment component 234. Other suitable configurations of the shaft 18 are contemplated.

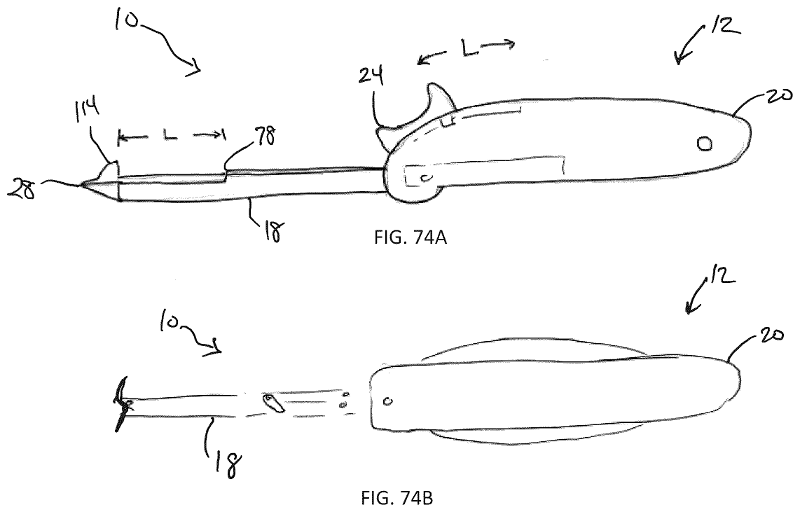

FIGS. 74A and 74B schematically depict views of an illustrative configuration of the device 10. FIG. 74A depicts a side view of the device 10, where the device 10 may include the shaft 18 extending distally from the handle 12, defining the opening 78, defining the garage 114, and defining the distal tip 28. The second actuator 24 may be configured to adjust a linear length L to adjust the blade a linear length L along the opening 78. Although the second actuator 24 may be depicted as a slider, the second actuator 24 may take on one or more other suitable configurations, as discussed herein or otherwise. FIG. 74B schematically depicts a bottom view of the device 10 depicted in FIG. 74A, with a distal end of the shaft 18 omitted.

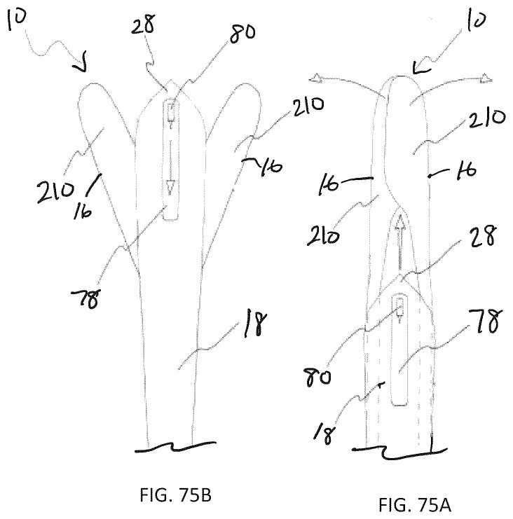

FIGS. 75A and 75B schematically depict views of an illustrative configuration of expanders 16 having a paddle 210 configuration, where the paddles 210 may be actuated with a longitudinal movement of the shaft 18. When the paddles 210 are in the closed position, as depicted in FIG. 75A, a distal end of the paddles 210 may be utilized as a probe during insertion and/or positioning of the device 10.

As depicted in FIG. 75B, the shaft 18 may be advanced distally relative to a position of the shaft 18 in FIG. 75A to engage the paddles 210. As the paddles 210 expand, tissue may be moved away from the opening 78 and the blade 80 may be adjusted from one or more protected positions to one or more exposed or cutting positions while mitigating the risk of cutting non-target tissue.

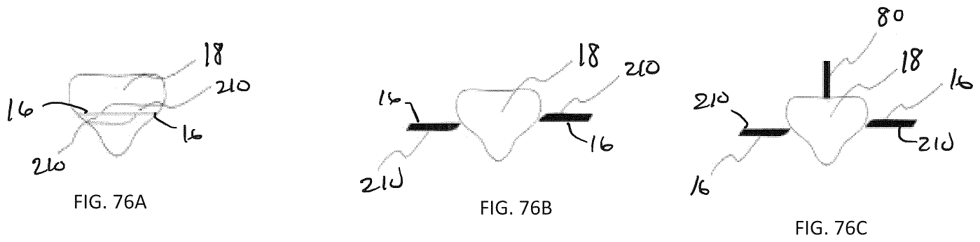

FIGS. 76A-76C schematically depict end views of the shaft 18 and the expanders 16 in the paddle 210 configuration. FIG. 76A schematically depicts the paddles 210 in a reduced diameter, probing configuration for insertion into the patient probing tissue within the patient. FIG. 76B schematically depicts the paddles 210 in an expanded configuration for pushing tissue away from the shaft 18. FIG. 76C schematically depicts the paddles in the expanded configuration and the blade 80 in an exposed or cutting position.

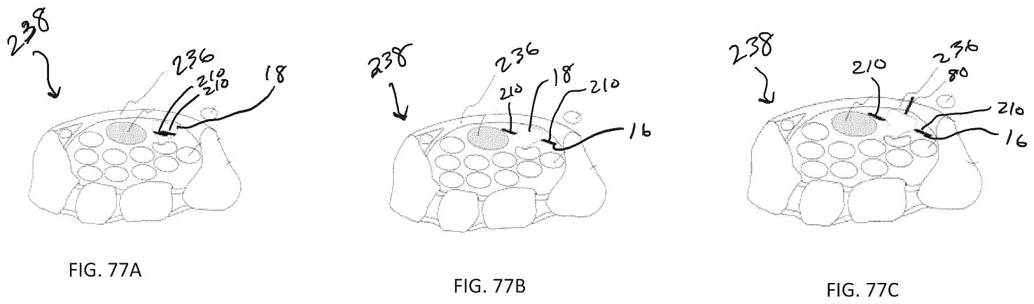

FIGS. 77A-77C schematically depict sectional views of the shaft 18 and the expanders 16 in the paddle 210 configuration, where the shaft 18 and the expanders 16 or the paddles 210 may be positioned proximate a transverse carpal ligament 236 at a wrist 238 of the patient for performing a carpal tunnel release procedure. FIG. 77A schematically depicts the expanders 16 or paddles 210 in a reduced diameter, probing configuration used for inserting the device 10 into the patient proximate (e.g., distal of) the transverse carpal ligament 236. Once the shaft 18 is positioned proximate the transverse carpal ligament 236, the expanders 16 or paddles 210 may be expanded to push tissue away from the shaft 18. Once a safe zone has been created around the shaft 18, the blade 80 may be actuated to an exposed or cutting position to cut through the transverse carpal ligament 236, as depicted in FIG. 77C. Once the transverse carpal ligament 236 is cut, the expanders 16 or paddles 210 may be returned to a reduced diameter position and the shaft 18 may be removed from the patient.

FIGS. 78A-78H depict views of a procedure with an illustrative configuration of the device 10. FIGS. 78A, 78B, and 78G schematically depict the expanders 16 or paddles 210 in a probing configuration and the distal tip 28 of the shaft 18 proximal of the expanders 16. FIG. 78C schematically depicts the shaft 18 advanced distally toward and over the expanders 16 or paddles 210, which expand the expanders 16 or paddles laterally outward to define a safe zone around the shaft 18. Once the safe zone has been established, the blade shaft 62 may be withdrawn and the blade 80 may travel along the guideway 82 to an exposed or cutting position, as depicted in FIGS. 78D and 78H. Further, the blade 80 may be withdrawn along the guideway 82 until the blade 80 travels from the exposed or cutting position to a proximal protected position, as depicted in FIG. 78E. With the blade 80 positioned at the proximal protected position, the shaft 18 may be withdrawn relative to the expanders 16 or paddles 210, as depicted in FIG. 78F, which may cause or allow the expanders 16 or paddles 210 to return to the probing configuration.

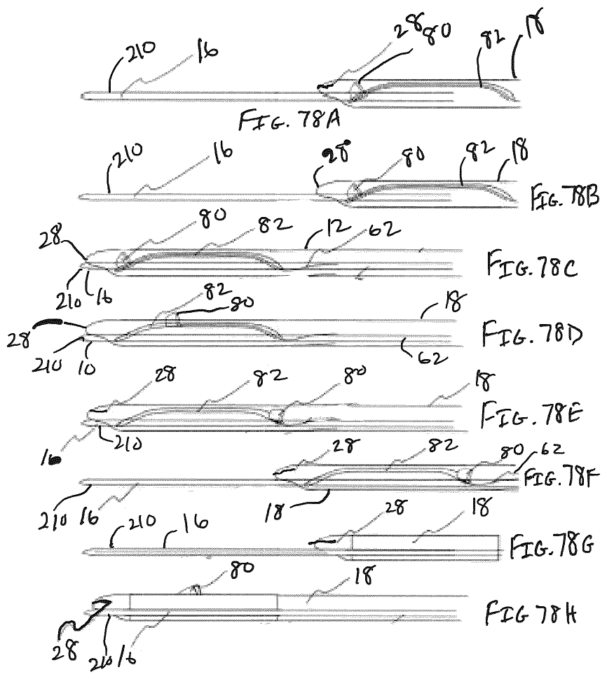

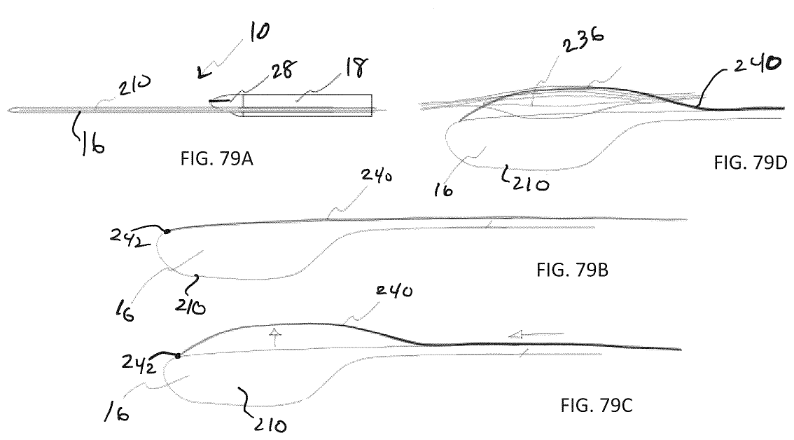

FIGS. 79A-79D schematically depict views of an illustrative technique for confirming soft tissue has been cut. FIG. 79A schematically depicts the expanders 16 or paddles 210 in a probing configuration and the distal tip 28 of the shaft 18 proximal of the expanders 16. FIG. 79B depicts the expanders 16 or paddles 210 in an expanded configuration and wire 240 (e.g., a k-wire or other suitable wire) extending along the expanders 16 or paddles 210, where the wire 240 may engage a distal stop 242 of the device 10. The wire 240 may be highly identifiable on ultrasound images and/or other images and may be used to determine whether a desired tissue has been fully cut. In operation, a distal force may be applied to the wire 240 while the wire 240 is engaging the distal stop 242, which may cause the wire to bow, as depicted for example in FIG. 79C. When a soft tissue, such as the transverse carpal ligament 236 or other suitable soft tissue, has been properly cut and the wire 240 is aligned with the cut (e.g., as depicted in ultrasound imaging), if the wire 240 bows without resistance when a distal force is applied to the wire 240, as depicted for example in FIG. 79D, the user may have confirmation that the soft tissue has been cut. If the wire 240 is aligned with the cut and receives resistance as the wire is bowing in response to the distal force applied to the wire 240, the user has confirmation that the soft tissue may not be cut to an extent desired and the cutting procedure may need to be repeated.

FIG. 80 schematically depicts an exploded view of an illustrative configuration of the device 10. As depicted in FIG. 80, the device 10 may include a fixed width expander 16 defining a channel 244. The channel 244 may be configured to receive a shuttle 246 configured to slidingly engage the expander 16 to be positioned in and/or adjust within or along the channel 244. The shuttle 246 may be coupled with a spring component 248 from which the blade 80 may extend. A fork component 250 may engage (e.g., slidingly engage or engage in one or more other suitable manner) the expander 16 to be positioned within or adjust within or along the channel 244, but other suitable configurations are contemplated. The fork component 250 may include two prongs 252 configured to engage spring levers 254 on the spring component 248. When the prongs 252 engage and/or disengage the spring levers 254, the blade 80 may adjust between a protected position and an exposed or cutting position.

FIGS. 81A and 81B schematically depict positioning of the fork component 250 relative to the spring component 248. FIG. 81A depicts the prongs 252 of the fork component 250 engaging the spring levers 254 of the spring component 248 to cause the blade 80 to be in an exposed or cutting position. FIG. 81B depicts the prongs 252 of the fork component 250 advanced distally of the spring levers 254 of the spring component 248 such that the prongs 252 are not engaging the spring levers 254 such that the spring component 248 biases the blade 80 to a protected position within the shuttle 246. Other suitable adjustment configurations are contemplated.

FIG. 82 schematically depicts a sectional view of an illustrative configuration of device 10 with the shuttle 246, the expanders 16, and the blade 80 in a protected position. The device 10 may have any suitable configuration and/or dimensions. For example, the shuttle 246 may have a width of about 3 mm, the shuttle 246 may have a height of about 6 mm, and the expanders 16 may have a width of about 8 mm. Other suitable configurations and/or dimensions are contemplated.

FIG. 83 schematically depicts a side view of an illustrative configuration of a blade 80 and a blade holder 256. The blade holder 256 and the blade 80 may be configured to pivot (e.g., about a longitudinally fixed pivot point and/or other suitable pivot point) within the shaft 18, the shuttle 246, and/or other suitable component proximate the distal end 10b of the device 10.

In some examples, the blade holder 256 may include an engagement component 258. The engagement component 258 may be configured to engage the prongs 252 of the fork component 250, the blade shaft 62, and/or other suitable component that may be adjusted to adjust a position of the blade holder 256 and/or the blade 80. As depicted in FIG. 83, the engagement component 258 may be a recess, but other suitable configurations of the engagement component 258 are contemplated including, but not limited to, a protrusion.

The blade holder 256 may be configured to facilitate movement of the blade 80 between a protected position and an exposed or cutting position in response to longitudinal movement or other movement of a component engaged with the engagement component 258. In some examples, a distal end or other portion of the blade holder 256 may define an angle that facilitates moving the blade 80 between a protected position and an exposed or cutting position as the blade holder 256 pivots. As depicted in FIG. 83, an angled surface 260 may extend from a base of the blade holder 256 distally and define an angle A3 with respect to a line or surface parallel to a longitudinal axis of the device 10 that may be configured to facilitate the blade 80 adjusting between the protected position and the cutting or exposed position. The angle A3 may have any suitable angle including, but not limited to, an angle in a range between about fifteen degrees and about seventy-five degrees, between about thirty degrees and about sixty degrees, and/or an angle in one or more other suitable ranges. In one example, the angle A3 may be about forty-five degrees.

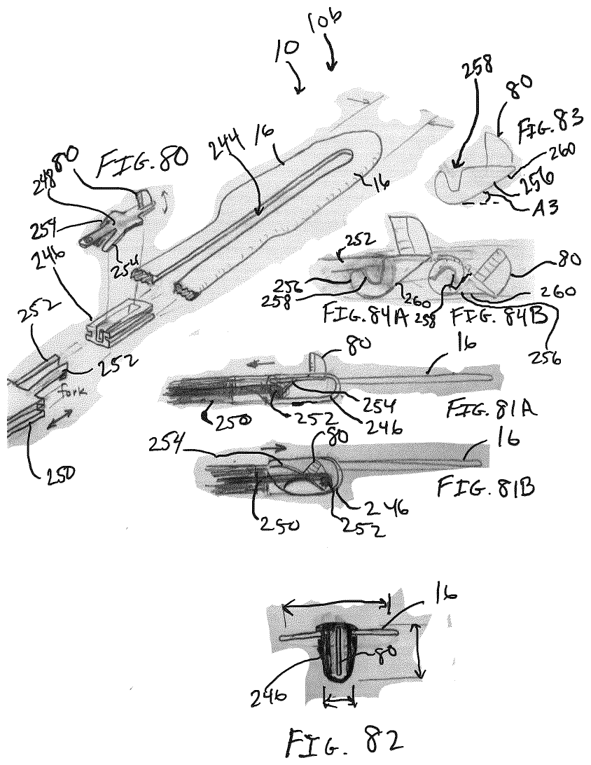

FIGS. 84A and 84B schematically depict movement of the blade 80 between the exposed or cutting position (e.g., as depicted in FIG. 84A) and a protected position (e.g., as depicted in FIG. 84B). As depicted in FIG. 84A, the prong 252 is engaging the engagement component 258 of the blade holder 256, where the prong 252 may be in a proximal position with the blade 80 in the exposed or cutting position. As depicted in FIG. 84B, the prong 252 remains engaging the engagement component 258 of the blade holder 256, where the prong 252 may advanced forward to a distal position with the blade 80 in the protected position and the surface 260 parallel to a longitudinal axis of the device 10. Other suitable configurations for adjusting the blade 80 in the blade holder 256 between the protected position and the exposed or cutting position are contemplated.

Unless otherwise expressly stated, it is in no way intended that any method or technique set forth herein is to be construed as requiring that its steps be performed in a specific order. This holds for any possible non-express basis for interpretation, including matters of logic with respect to arrangement of steps or operational flow, plain meaning derived from grammatical organization or punctuation, and the number or type of embodiments described in the specification

This disclosure is, in many respects, only illustrative. Changes may be made in details, particularly in matters of shape, size, and arrangement of steps without exceeding the scope of the disclosure. The invention's scope is, of course, defined in the language in which the appended claims are expressed.

What is claimed is:

1. A device for cutting body tissue, comprising:
a handle, comprising:
a first actuator; and
a second actuator in communication with the first actuator;
a blade coupled with the second actuator;
a shaft extending distally from the handle, the shaft defining a guideway along which the blade is configured to travel; and
an expander in communication with the first actuator and extending along the shaft,
wherein actuation of the first actuator adjusts the expander to an expanded position and unlocks the second actuator to allow actuation of the second actuator and longitudinal movement of the blade along the guideway.

2. The device of claim 1, wherein in response to forces applied to the first actuator, the first actuator is configured to adjust between an unactuated position in which the expander is in a relaxed position and a fully actuated position in which the expander is in the expanded position.

3. The device of claim 2, wherein the first actuator is biased toward the unactuated position and the first actuator is configured to remain in the fully actuated position after the first actuator is adjusted to the fully actuated position and the forces applied to the first actuator are removed.

4. The device of claim 1, further comprising:
an expansion system at least partially located in the handle and in communication with the first actuator and the expander, wherein the expansion system is configured to cause the expander to expand in response to actuation of the first actuator.

5. The device of claim 4, wherein the expansion system comprises a expansion device, a manifold in communication with the expansion device, and tubing in communication with the manifold and the expander, the expansion device being configured to cause fluid to flow through the manifold and the tubing to the expander in response to actuation of the first actuator.

6. The device of claim 4, wherein the handle comprises a priming port in fluid communication with the expansion system and configured to engage a fluid source, the priming port including a one-way valve configured to open in response to the priming port engaging with the fluid source.

7. The device of claim 4, further comprising a linkage coupled with the first actuator and second actuator.

8. The device of claim 7, wherein the linkage is coupled to the first actuator with an over-center mechanism configured to secure the first actuator in a fully actuated position until a force is applied to the first actuator to overcome the over-center mechanism.

9. The device of claim 1, further comprising:
a linkage extending between the first actuator and the first actuator, and
wherein the linkage disengages from the second actuator when the first actuator is in a fully actuated position to unlock the second actuator and allow actuation of the second actuator to longitudinally move the blade along the guideway.

10. The device of claim 1, wherein the guideway comprises a first portion with an incline, a second portion extending proximally from the first portion, and a third portion extending proximally from the second portion and having a decline.

11. The device of claim 1, wherein the shaft has an elongate polymer portion defining the guideway and an elongate metal portion defining the guideway.

12. The device of claim 1, wherein the second actuator is a second actuator and has a first extension and a second extension spaced proximally from the first extension.

13. A method of operating a device for cutting body tissue, the method comprising:
actuating a first actuator to radially enlarge an expander extending along a guideway for a blade of the device for cutting body tissue and unlock a second actuator coupled with the blade; and
adjusting the second actuator to move the blade from a protected first position to a cutting second position.

14. The method of claim 13, wherein the expander is a balloon, and the method further comprises:
priming an expansion system with fluid, and
wherein actuating the first actuator actuates an expansion device of the expansion system causing the fluid to travel to the balloon and radially enlarge the balloon.

15. The method of claim 14, wherein priming the expansion system with fluid comprises:
engaging a fluid source with a priming port of the device for cutting body tissue;
creating a vacuum pressure in the expansion system and the fluid source by withdrawing a plunger of the fluid source; and
releasing the plunger.

16. The method of claim 13, further comprising:
adjusting the second actuator to move the blade from the cutting second position to a protected third position.

17. A handle assembly of a device for cutting body tissue, the handle assembly comprising:
a lever;
an expansion system in communication with the lever;
a slider configured to couple with a blade and longitudinally adjust the blade as the slider is longitudinally adjusted; and
a linkage coupled with the lever and configured to limit movement of the slider, and
wherein when the lever is adjusted to a fully actuated position, the linkage is configured unlock the slider and allow the slider to adjust longitudinally.

18. The handle assembly of claim 17, further comprising:
a priming port in fluid communication with the expansion system.

19. The handle assembly of claim 18, wherein the expansion system comprises:
a manifold in communication with the priming port;
tubing in fluid communication with the manifold; and
an expansion device configured to move fluid through the manifold and the tubing in response to adjustments of the lever.

20. The handle assembly of claim 19, wherein the manifold comprises a pressure relief valve and a pressure relief chamber.
